(19) United States
(12) Patent Application Publication
(10) Pub. No.: US 2021/0330638 A1
(43) Pub. Date: Oct. 28, 2021

(54) NANO-PENETRATIVE CANNABINOID OIL BLENDS AND COMPOSITIONS AND METHODS OF FORMULATION THEREOF

(71) Applicant: Theragun, Inc., Los Angeles, CA (US)

(72) Inventor: Timothy Drennan, Denver, CO (US)

(21) Appl. No.: 17/327,234

(22) Filed: May 21, 2021

Related U.S. Application Data
(63) Continuation of application No. PCT/US2021/019114, filed on Feb. 22, 2021.
(60) Provisional application No. 62/979,924, filed on Feb. 21, 2020.
(60) Provisional application No. 62/979,917, filed on Feb. 21, 2020.

(51) Int. Cl. A61K 31/352; A61K 31/05; A61K 31/085; A61K 31/045; A61K 31/122; A61K 31/015; A61K 31/125; A61K 36/61; A61K 36/534; A61K 9/00; A61K 9/10; A61P 29/02

(52) U.S. Cl. CPC A61K 31/352; A61K 31/05; A61K 31/085; A61K 31/045; A61K 31/122; A61K 31/015; A61P 29/02; A61K 36/61; A61K 36/534; A61K 9/0014; A61K 9/0056; A61K 9/10; A61K 31/125

(57) ABSTRACT

The present disclosure relates to methods of formulating therapeutic oil blends comprising one or more terpenes, wherein the methods may result in blends exhibiting increased stability and bioavailability. The methods may be used to produce compositions for dermal or transdermal application useful in the treatment of, e.g., pain. Disclosed methods may also result in improved homogeneity and non-crystallization of the formulated products.

### CROSS-REFERENCE TO RELATED APPLICATIONS

This application is a continuation of International Application No. PCT/US2021/019114, filed on Feb. 22, 2021, which claims the benefit of priority to U.S. Provisional Application No. 62/979,917, filed on Feb. 21, 2020, and to U.S. Provisional Application No. 62/979,924, filed on Feb. 21, 2020, the contents of each of which are herein incorporated by reference in their entireties.

### FIELD OF THE DISCLOSURE

The present disclosure relates to therapeutic oil blends comprising one or more cannabinoids and one or more terpenes, wherein the therapeutic oil blends may exhibit increased stability and bioavailability, and methods of formulation thereof. The methods may be used to improve one or more properties of particle size, nano-penetration, and non-crystallization. The resulting therapeutic oil blends and compositions comprising may be useful for the treatment of various conditions, such as pain and psychological conditions. Also disclosed are compositions comprising the therapeutic oil blends and methods of treatment comprising administering therapeutic oil blends and compositions for the treatment of various conditions, such as pain and psychological conditions.

### BACKGROUND

Cannabis is one of the most widely used herbs for medicinal purposes. Medical cannabis is used for treating and alleviating symptoms associated with a growing number of indications, including pain, anorexia, asthma, glaucoma, arthritis, spasms, anxiety, and substance withdrawal. Many other illnesses are emerging as potential cannabis-responsive indications. Cannabidiol (CBD) is a phytocannabinoid, one of the most plentiful of the numerous cannabinoids derived from Cannabis plants. CBD is non-psychotropic, but has biochemical and pharmacological features that make it relevant for numerous therapeutic applications. Existing studies have explored the use of CBD in treating, for example, neurological disorders, seizures, anxiety, diabetes, nausea, arthritis and even cancer. CBD has also been used for thousands of years to treat various forms of pain.

However, many existing cannabinoid-based compositions face issues of limited shelf-life due to crystallization. Some blends crystallize in a period as short as 24 hours. This limits the ability to implement such compositions in ready-to-use applications, as they require heat and homogenization prior to use. In addition, topical application and oral administration of cannabinoids have been limited by problems with bioavailability. Existing hemp oils on the market are largely distillates, with some isolates and raw crude oils also being available. The average particle size of these compositions is around 2,000 nanometers. However, human skin, for example, can only efficiently make use of smaller sized particles in the sub 0.1 μm range. The large particle size of presently available compositions not only poses a problem for bioavailability, but also makes hemp oils more difficult to use downstream in other formulations.

There is a long unmet need for methods of formulating cannabinoid compositions with improved stability and bioavailability for numerous applications.

### BRIEF SUMMARY OF THE INVENTION

In one aspect, the present disclosure provides a mechanically agitated, non-crystallizing liquid therapeutic oil blend with emulsion-like properties comprising: a) 30-90% w/w cannabinoid oil; and b) 25-75% w/w of one or more essential oils.

In one aspect, the present disclosure provides a cavitated, non-crystallizing liquid therapeutic oil blend with emulsion-like properties comprising: a) 30-90% w/w cannabinoid oil; and b) 25-75% w/w of one or more essential oils.

In one aspect, the present disclosure provides a sonicated, non-crystallizing liquid therapeutic oil blend with emulsion-like properties comprising: a) 30-90% w/w cannabinoid oil; and b) 25-75% w/w of one or more essential oils.

In one aspect, the present disclosure provides a liquid therapeutic oil blend that does not crystallize at room temperature for a period of at least 60 days comprising: a) 30-90% w/w cannabinoid oil; and b) 25-75% w/w of one or more essential oils.

In one aspect, the present disclosure provides a liquid therapeutic oil blend with emulsion-like properties that has an average particle size of less than 0.1 μM comprising: a) 30-90% w/w cannabinoid oil; and b) 25-75% w/w of one or more essential oils.

In one aspect, the present disclosure provides a mechanically agitated, non-crystallizing liquid therapeutic oil blend with emulsion-like properties comprising: a) 25-75% w/w cannabinoid, and b) 25-75% w/w terpenes.

In one aspect, the present disclosure provides a cavitated, non-crystallizing liquid therapeutic oil blend with emulsion-like properties comprising: a) 25-75% w/w cannabinoid, and b) 25-75% w/w terpenes.

In one aspect, the present disclosure provides a sonicated, non-crystallizing liquid therapeutic oil blend with emulsion-like properties comprising: a) 25-75% w/w cannabinoid, and b) 25-75% w/w terpenes.

In one aspect, the present disclosure provides a liquid therapeutic oil blend that does not crystallize at room temperature for a period of at least 60 days comprising: a) 25-75% w/w cannabinoid, and b) 25-75% w/w terpenes.

In one aspect, the present disclosure provides a liquid therapeutic oil blend with emulsion-like properties that has an average particle size of less than 0.1 μM comprising: a) 25-75% w/w cannabinoid, and b) 25-75% w/w terpenes.

In one aspect, the present disclosure provides a mechanically agitated, non-crystallizing liquid therapeutic oil blend with emulsion-like properties comprising: a) 30-90% w/w cannabinoid oil; b) 15-40% w/w clove bud oil; and c) 5-20% w/w peppermint oil.

In one aspect, the present disclosure provides a cavitated, non-crystallizing liquid therapeutic oil blend with emulsion-like properties comprising: a) 30-90% w/w cannabinoid oil; b) 15-40% w/w clove bud oil; and c) 5-20% w/w peppermint oil.

In one aspect, the present disclosure provides a sonicated, non-crystallizing liquid therapeutic oil blend with emulsion-like properties comprising: a) 30-90% w/w cannabinoid oil; b) 15-40% w/w clove bud oil; and c) 5-20% w/w peppermint oil.

In one aspect, the present disclosure provides a liquid therapeutic oil blend that does not crystallize at room temperature for a period of at least 60 days comprising: a) 30-90% w/w cannabinoid oil; b) 15-40% w/w clove bud oil; and c) 5-20% w/w peppermint oil.

In one aspect, the present disclosure provides a liquid therapeutic oil blend with emulsion-like properties that has an average particle size of less than 0.1 μM comprising: a) 30-90% w/w cannabinoid oil; b) 15-40% w/w clove bud oil; and c) 5-20% w/w peppermint oil.

In some embodiments, the therapeutic oil blend has been cavitated.

In some embodiments, the therapeutic oil blend has been sonicated.

In some embodiments, the therapeutic oil blend is homogeneous.

In some embodiments, the therapeutic oil blend is liquid at room temperature.

In some embodiments, the therapeutic oil blend is liquid at room temperature for a period of at least 60 days.

In some embodiments, the therapeutic oil blend is shelf stable.

In some embodiments, the therapeutic oil blend is shelf stable for a period of at least 60 days.

In some embodiments, the therapeutic oil blend comprises 30-90% w/w of the cannabinoid oil.

In some embodiments, the therapeutic oil blend comprises 25-75% w/w cannabinoid.

In some embodiments, the cannabinoid oil comprises cannabidiol (CBD).

In some embodiments, the cannabinoid oil comprises cannabidiolic acid (CBDA).

In some embodiments, the cannabinoid oil comprises cannabidiol monomethylether (CBDM).

In some embodiments, the cannabinoid oil comprises cannabidiol-C4 (CBD-C4).

In some embodiments, the cannabinoid oil comprises cannabidivarinic acid (CBDVA).

In some embodiments, the cannabinoid oil comprises cannabidivarin (CBDV).

In some embodiments, the cannabinoid oil comprises cannabidiorcol (CBD-C1).

In some embodiments, the cannabinoid oil comprises delta 9 tetrayhydrocannabinol (THC).

In some embodiments, the therapeutic oil blend comprises 25-30%, 30-35%, 35-40%, 40-45%, 45-50%, 50-55%, 55-60%, 60-65%, 65-70%, or 70-75% w/w cannabinoids.

In some embodiments, the therapeutic oil blend comprises 25-30%, 30-35%, 35-40%, 40-45%, 45-50%, 50-55%, 55-60%, 60-65%, 65-70%, or 70-75% w/w CBD.

In some embodiments, the cannabinoid oil comprises hemp oil.

In some embodiments, the cannabinoid oil comprises a cannabinoid isolate.

In some embodiments, the cannabidiol oil comprises a cannabis extract.

In some embodiments, the cannabinoid oil comprises at least 30%, 40%, 50%, 60%, 70%, or 80% cannabinoids.

In some embodiments, the cannabinoid oil comprises at least 30%, 40%, 50%, 60%, 70%, or 80% CBD.

In some embodiments, the one or more essential oils are selected from the group consisting of Linalool; B-Caryophyllene; B-Myrcene; D-Limonene; Humulene; a-Pinene; Ylang Ylang; Yarrow; Violet; Vetiver; Vanilla; Tuberose; Thyme; Tea Tree; Tangerine; Spruce, Black; Spruce; Spikenard; Spearmint; Sandalwood; Rosewood; Rosemary Verbenone; Rosemary; Rose; Rose Geranium; Ravensara; Plai Pine Needle; Petitgrain; Peppermint; Pepper, Black; Patchouli; Palo Santo; Palmarosa; Osmanthus; Oregano; Orange, Sweet; Oak Moss; Nutmeg Niaouli; Neroli; Myrtle; Myrrh; Mimosa; Melissa; Marjoram, Sweet; Manuka; Mandarin, Red; Mandarin; Lotus, White; Lotus, Pink; Lotus, Blue; Lime; Lily; Lemongrass; Lemon; Lavender; Lavandin; Kanuka; Juniper Berry; Jasmine; Jasmine Abs; Helichrysum; Grapefruit, White; Grapefruit, Pink; Ginger; Geranium; Geranium, Bourbon; Gardenia; Galbanum; Frankincense; Frangipani; Fir Needle White; Fir Needle Siberia; Fir Needle Canada; Fennel, Sweet; Eucalyptus Smithii. Eucalyptus Radiata, Eucalyptus Globulus, Eucalyptus Citriodora, Eucalyptus Blue Mallee; Elemi; Dill; Cypress; Cumin; Coriander; Cocoa; Clove; Clary Sage; Cistus; Cinnamon; Chamomile, Roman; Chamomile, Blue; Celery Seed; Cedarwood, Western Red; Cedarwood, Blood; Cedarwood Atlas; Carrot Seed; Cardamon; Caraway Seed; Cajeput; Cade; Birch, White; Birch, Sweet; Bergamot; Bay Laurel; Basil; Basil, Holy; Basil; Balsam Poplar; Balsam Peru; Angelica; and combinations thereof.

In some embodiments, the one or more essential oils are obtained from one or more plants or plant parts selected from the group consisting of Alfalfa; Allspice; Almont, bitter; Ambrette; Angelica root; Angelica seed; Angelica stem; Angostura; Anise; Asafetida; Balm; Balsam of Peru; Basil; Bay leaves; Bay; Bergamot; Bitter almond; Bois de rose; Cacao; Camomile flowers, Hungarian; Camomile flowers, Roman or English; Cananga; Capsicum; Caraway; Cardamom seed; Carob bean; Carrot; Cascarilla bark; Cassia bark, Chinese; Cassia bark, Padang or Batavia; Cassia bark, Saigon; Celery seed; Cherry, wild, bark; Chervil; Chicory; Cinnamon bark, Ceylon; Cinnamon bark, Chinese; Cinnamon bark, Saigon; Cinnamon leaf, Ceylon; Cinnamon leaf, Chinese; Cinnamon leaf, Saigon; Citronella; Citrus peels; Clary; Clover; Coca; Coffee; Cola nut; Coriander; Cumin; Curacao orange peel; Cusparia bark; Dandelion; Dandelion root; Dog grass; Elder flowers; Estragole; Estragon; Fennel, sweet; Fenugreek; Galanga; Geranium; Geranium, East Indian; Geranium, rose; Ginger; Grapefruit; Guava; Hickory bark; Horehound; Hops; Horsemint; Hyssop; Immortelle; Jasmine; Juniper; Kola nut; Laurel berries; Laurel leaves; Lavender; Lavender, spike; Lavandin; Lemon; Lemon balm; Lemon grass; Lemon peel; Lime; Linden flowers; Locust bean; Lupulin; Mace; Mandarin; Marjoram, sweet; Mate; Melissa; Menthol; Menthyl acetate; Molasses; Mustard; Naringin; Neroli, bigarade; Nutmeg; Onion; Orange, bitter, flowers; Orange, bitter, peel; Orange leaf; Orange, sweet; Orange, sweet, flowers; Orange, sweet, peel; Origanum; Palmarosa; Paprika; Parsley; Pepper, black; Pepper, white; Peppermint; Peruvian balsam; Petitgrain; Petitgrain lemon; Petitgrain mandarin or tangerine; Pimenta; Pimenta leaf; Pipsissewa leaves; Pomegranate; Prickly ash bark; Rose absolute; Rose; Rose buds; Rose flowers; Rose fruit; Rose geranium; Rose leaves; Rosemary; Saffron; Sage; Sage, Greek; Sage, Spanish; St. John's bread; Savory, summer; Savory, winter; Schinus molle; Sloe berries; Spearmint; Spike lavender; Tamarind; Tangerine; Tarragon; Tea; Thyme; Thyme, white; Thyme, wild or creeping; Triticum; Tuberose; Turmeric; Vanilla; Violet flowers; Violet leaves; Violet leaves absolute; Wild cherry bark; Ylang-ylang; Zedoary bark; and combinations thereof.

In some embodiments, the one or more essential oils comprise clove bud oil and peppermint oil.

In some embodiments, the therapeutic oil blend comprises about 15-40% w/w clove bud oil.

In some embodiments, the therapeutic oil blend comprises about 5-20% w/w peppermint oil.

In some embodiments, the therapeutic oil blend comprises about 15-40% w/w clove bud oil and 5-20% w/w peppermint oil.

In some embodiments, the therapeutic oil blend does not comprise any exogenously added emulsifiers, surfactants, or other homogenizing agents, other than the terpenes.

In some embodiments, the therapeutic oil blend is not an emulsion.

In some embodiments, the therapeutic oil blend does not comprise a water phase.

In some embodiments, the therapeutic oil blend comprises less than 20% water-soluble ingredients.

In some embodiments, the therapeutic oil blend comprises less than 5% water-soluble ingredients.

In some embodiments, the therapeutic oil blend does not comprise any emulsifiers, surfactants, or other homogenizing agents, other than those naturally occurring in hemp oil, clove bud oil, and peppermint oil.

In some embodiments, the therapeutic oil blend has an average particle size of less than 1 μm.

In some embodiments, the therapeutic oil blend has an average particle size of less than 0.1 μm.

In some embodiments, the therapeutic oil blend has an average particle size of about 20-60 nm.

In some embodiments, the therapeutic oil blend is nano-penetrative.

In some embodiments, the therapeutic oil blend penetrates the sensory nerve fibers faster than a corresponding control formulation without the essential oil or terpene component or a corresponding control formulation without the mechanical agitation employed in preparing the therapeutic oil blend.

In some embodiments, the therapeutic oil blend has improved topical absorption compared to a corresponding control formulation without the essential oil or terpene component or a corresponding control formulation without the mechanical agitation employed in preparing the therapeutic oil blend.

In some embodiments, the therapeutic oil blend has anesthetic properties.

In some embodiments, the therapeutic oil blend has anesthetic properties greater than the anesthetic properties of a corresponding control formulation without the essential oil or terpene component.

In some embodiments, the therapeutic oil blend has anesthetic properties greater than the anesthetic properties of a mixture of the components without the mechanical agitation employed in preparing the therapeutic oil blend.

In some embodiments, a topical application of the therapeutic oil blend results in a decrease in painful stimulus at the site of application for a period of at least ten minutes following application.

In some embodiments, the therapeutic oil blend has analgesic properties.

In some embodiments, the therapeutic oil blend has vasodilatory effects.

In some embodiments, topical application of the therapeutic oil blend increases skin temperature at the site of application by at least 0.5° C. over the course of one minute following application.

In some embodiments, topical application of the therapeutic oil blend increases skin temperature at the site of application by at least 1.0° C. over the course of one minute following application.

In some embodiments, the therapeutic oil blend has a faster solvation rate in a carrier oil than solid or liquid hemp oil alone.

In some embodiments, the therapeutic oil blend has a faster solvation rate in MCT oil than solid or liquid hemp oil alone.

In some embodiments, the therapeutic oil blend comprises about 20%-40% w/w eugenol.

In some embodiments, the therapeutic oil blend comprises about 5%-20% w/w menthol and/or menthone; and In some embodiments, the therapeutic oil blend comprises about 0.1%-10% w/w d-limonene.

In some embodiments, the therapeutic oil blend comprises about 0.01%-5% w/w of one or more terpenes selected from the list consisting of bisabolol, borneol, caryophyllene, carene, camphene, camphor, cineol, citronellal, eucalyptol, geraniol, guaiol, humulene, isopropyltoluene, isopulegol, linalool, beta-myrcene, nerolidol, ocimene, alpha-pinene, beta-pinene, phytol, pulegone, alpha-terpinene, gamma-terpinene, terpinolene, thymol.

In some embodiments, the therapeutic oil blend is formulated for transdermal penetration and/or membrane penetration.

In some embodiments, the therapeutic oil blend is non-crystallizing.

In some embodiments, the therapeutic oil blend is non-crystallizing for a period of at least 30 days.

In some embodiments, the therapeutic oil blend has improved bioavailability, consistency, shelf life, and non-crystallization compared to the cannabinoid oil alone.

In one aspect, the present disclosure provides a mechanically agitated, non-crystallizing liquid therapeutic oil blend with emulsion-like properties comprising: a) about 63% w/w cannabinoid oil; b) about 27% w/w clove bud oil; and c) about 10% w/w peppermint oil.

In one aspect, the present disclosure provides a mechanically agitated, non-crystallizing liquid therapeutic oil blend with emulsion-like properties comprising: a) about 50% w/w cannabidiol (CBD); b) about 20-25% w/w eugenol; c) about 8-10% w/w menthol and menthone; and d) about 1-3% w/w d-limonene.

In one aspect, the present disclosure provides a topical formulation comprising the therapeutic oil blend of any one of the foregoing embodiments.

In some embodiments, the topical formulation is a lotion.

In some embodiments, the topical formulation is a body balm.

In some embodiments, the topical formulation is a salve.

In some embodiments, the topical formulation is a massage oil.

In some embodiments, the topical formulation is a topical gel.

In some embodiments, the topical formulation is a moisturizer.

In some embodiments, the topical formulation is a cream.

In some embodiments, the topical formulation comprises an essential oil.

In some embodiments, the topical formulation comprises a carrier oil.

In some embodiments, the topical formulation comprises a humectant.

In some embodiments, the topical formulation comprises an emulsifier.

An ingestible formulation comprising the therapeutic oil blend of any one of the foregoing embodiments.

In some embodiments, the ingestible formulation is a tablet.

In some embodiments, the ingestible formulation is a pressed tablet.

In some embodiments, the ingestible formulation is a tincture.

In some embodiments, the ingestible formulation is a drink mix.

In some embodiments, the ingestible formulation comprises an essential oil.

In some embodiments, the ingestible formulation comprises a carrier oil.

In one aspect, the present disclosure provides a method of using a therapeutic oil blend according to any of embodiment 1-77, or composition comprising, in the treatment of a condition.

In one aspect, the present disclosure provides a method of using a topical formulation according to any one of the foregoing embodiments or an ingestible formulation according to any one of the foregoing embodiments in the treatment of a condition.

In some embodiments, the condition is pain.

In some embodiments, the condition is poor sleep quality.

In some embodiments, the condition is fatigue.

In some embodiments, the condition is a psychological condition.

In one aspect, the present disclosure provides a hermetically sealed container comprising the therapeutic oil blend any one of the foregoing embodiments, or a composition comprising.

In one aspect, the present disclosure provides a method of formulating a therapeutic oil blend according to any one of the foregoing embodiments.

In one aspect, the present disclosure provides a method for preparing a mechanically agitated, non-crystallizing therapeutic oil blend with emulsion-like properties, the method comprising the steps of: a) providing a cannabinoid oil, a clove bud oil, and a peppermint oil ingredients; b) admixing the ingredients of step (a), thereby forming an inactive mixture; and c) mechanically agitating the inactive mixture of step (b) until homogeneous, thereby producing the mechanically agitated therapeutic oil blend.

In one aspect, the present disclosure provides a method for preparing a sonicated, non-crystallizing therapeutic oil blend with emulsion-like properties, the method comprising the steps of: a) providing a cannabinoid oil, a clove bud oil, and a peppermint oil ingredient; b) admixing the ingredients of step (a), thereby forming an inactive mixture; and c) sonicating the inactive mixture of step (b) until homogeneous, thereby producing the sonicated therapeutic oil blend.

In one aspect, the present disclosure provides a method for preparing a mechanically agitated therapeutic oil blend that is liquid at room temperature and does not crystallize at room temperature for a period of at least 60 days, the method comprising the steps of: a) providing a cannabinoid oil, a clove bud oil, and a peppermint oil ingredient; b) admixing the ingredients of step (a), thereby forming an inactive mixture; and c) mechanically agitating the inactive mixture of step (b) until homogeneous, thereby producing the mechanically agitated therapeutic oil blend.

In one aspect, the present disclosure provides a method for preparing a mechanically agitated therapeutic oil blend with emulsion-like properties that has an average particle size of less than 0.1 μM, the method comprising the steps of: a) providing a cannabinoid oil, a clove bud oil, and a peppermint oil ingredient; b) admixing the ingredients of step (a), thereby forming an inactive mixture; and c) mechanically agitating the inactive mixture of step (b) until homogeneous, thereby producing the mechanically agitated therapeutic oil blend.

In one aspect, the present disclosure provides a method for preparing a mechanically agitated, non-crystallizing therapeutic oil blend with emulsion-like properties, the method comprising the steps of: a) providing a cannabinoid oil, eugenol, menthol, menthone, and d-limonene ingredients; b) admixing the ingredients of step (a), thereby forming an inactive mixture; and c) mechanically agitating the inactive mixture of step (b) until homogeneous, thereby producing the mechanically agitated therapeutic oil blend.

In one aspect, the present disclosure provides a method for preparing a sonicated, non-crystallizing therapeutic oil blend with emulsion-like properties, the method comprising the steps of: a) providing a cannabinoid oil, eugenol, menthol, menthone, and d-limonene ingredient; b) admixing the ingredients of step (a), thereby forming an inactive mixture; and c) sonicating the inactive mixture of step (b) until homogeneous, thereby producing the sonicated therapeutic oil blend.

In one aspect, the present disclosure provides a method for preparing a mechanically agitated therapeutic oil blend that is liquid at room temperature and does not crystallize at room temperature for a period of at least 60 days, the method comprising the steps of: a) providing a cannabinoid oil, eugenol, menthol, menthone, and d-limonene ingredient; b) admixing the ingredients of step (a), thereby forming an inactive mixture; and c) mechanically agitating the inactive mixture of step (b) until homogeneous, thereby producing the mechanically agitated therapeutic oil blend.

In one aspect, the present disclosure provides a method for preparing a mechanically agitated therapeutic oil blend with emulsion-like properties that has an average particle size of less than 0.1 μM, the method comprising the steps of: a) providing a cannabinoid oil, eugenol, menthol, menthone, and d-limonene ingredient; b) admixing the ingredients of step (a), thereby forming an inactive mixture; and c) mechanically agitating the inactive mixture of step (b) until homogeneous, thereby producing the mechanically agitated therapeutic oil blend.

In some embodiments, the cannabinoid oil ingredient has all of its cannabinoid contents fully dissolved before the admixing step.

In some embodiments, the cannabinoid oil ingredient has been been cavitated before the admixing step.

In some embodiments, the cannabinoid oil is heated until fully liquified prior to admixing or prior to mechanical agitation.

In some embodiments, the cannabinoid oil is heated to at least 60° C. prior to admixing or prior to mechanical agitation.

In some embodiments, the method comprises mechanically agitating the cannabinoid oil ingredient prior to step (b).

In some embodiments, the method further comprises the following steps prior to step (b): i) heating the cannabinoid oil to at least 60° C. until fully liquified, and ii) mechanically agitating the liquified cannabinoid oil.

In some embodiments, the cannabinoid oil is mechanically agitated until cavitation is induced prior to step (b).

In some embodiments, step (b) comprises mechanically agitating the inactive mixture until a sufficient level of cavitation is induced so as to fully homogenize the mixture.

In some embodiments, the temperature of the cannabinoid oil does not exceed about 80° C. for more than 10, 20, 30, 40, 50, 60, 70, 80, 90 or 100 minutes during any step carried out on the cannabinoid oil alone.

In some embodiments, the inactive mixture is maintained below a temperature of about 60° C. throughout step (c).

In some embodiments, the inactive mixture is maintained below a temperature of about 55° C. throughout step (c).

In some embodiments, the inactive mixture is maintained below a temperature of about 40° C. throughout step (c).

In some embodiments, the ingredients other than the cannabinoid oil are chilled prior to step (c).

In some embodiments, the ingredients other than the cannabinoid oil are chilled to about less than 0° C. prior to step (c).

In some embodiments, the cannabinoid oil is at room temperature prior to adding other ingredients in step (b).

In some embodiments, step (b) and (c) are carried out such that the inactive mixture remains at or below 25° C.

In some embodiments, step (b) and (c) comprise sequentially adding each ingredient to the cannabinoid oil, with a round of mechanical agitation in between the addition of each ingredient.

In some embodiments, the eugenol or the clove bud oil is added to the cannabinoid oil, thereby producing a eugenol mixture, prior to addition of the other ingredients in step (b).

In some embodiments, the eugenol mixture is mechanically agitated prior to addition of the other ingredients in step (b).

In some embodiments, the temperature of the eugenol mixture is maintained below about 60° C. during mechanical agitation.

In some embodiments, the eugenol or clove bud oil is added to the cannabinoid oil while the cannabinoid oil is experiencing mechanical agitation.

In some embodiments, the eugenol or clove bud oil is added to the cannabinoid oil while the cannabinoid oil is experiencing sonication.

In some embodiments, the mechanical agitation is carried out at settings selected to avoid vaporization, sublimation, or denaturation of the eugenol.

In some embodiments, the other ingredients are added to the mechanically agitated eugenol mixture, thereby producing an overall mixture.

In some embodiments, the mechanically agitated eugenol mixture is chilled to less than about 15° C. prior to addition of the remaining ingredients.

In some embodiments, the other ingredients are chilled prior to being added to the mechanically agitated eugenol mixture.

In some embodiments, the temperature of the other ingredients and the temperature of the mechanically agitated eugenol mixture are such that the temperature of the inactive mixture is kept below about 15° C.

In some embodiments, the inactive mixture is mechanically agitated.

In some embodiments, the temperature of the inactive mixture is maintained below about 40° C. during mechanical agitation.

In some embodiments, the other ingredients are added to the eugenol mixture to form an inactive mixture during the process of mechanical agitation.

In some embodiments, steps (b) and (c) are conducted simultaneously.

In some embodiments, the mechanical agitation is carried out at settings selected to avoid vaporization, sublimation, or denaturation of the eugenol and other ingredients.

In some embodiments, d-limonene is added to the eugenol mixture prior to the addition of menthol and menthone.

In some embodiments, following step (c), the inactive mixture is further mechanically agitated.

In some embodiments, the temperature does not exceed about 55° C. during further mechanical agitation.

In some embodiments, the mechanical agitation step is carried out until homogeneity is achieved.

In some embodiments, the mechanical agitation induces cavitation.

In some embodiments, the mechanical agitation is sonication.

In one aspect, the present disclosure provides a method for preparing a mechanically agitated, non-crystallizing, homogeneous therapeutic oil blend with emulsion-like properties, wherein the therapeutic oil blend comprises 25-75% w/w cannabinoid; 15%-40% w/w eugenol; 5%-20% w/w menthol and/or menthone; and 0.1%-10% w/w d-limonene, the method comprising the steps of: a) providing a cannabinoid oil comprising the cannabinoid, heating said cannabinoid oil to at least about 60° C., and sonicating said cannabinoid oil to induce cavitation; b) adding the eugenol to the sonicated cannabinoid oil of (a) and sonicating until the mixture is fully homogenized; c) adding the d-limonene, menthol, and/or menthone to the mixture of (b) and sonicating until the mixture is fully homogenized; d) sonicating the mixture of (c) until the solution has the desired particle size, thereby preparing the therapeutic oil blend.

In one aspect, the present disclosure provides a method for preparing a mechanically agitated, non-crystallizing, homogeneous therapeutic oil blend with emulsion-like properties, wherein the therapeutic oil blend comprises 25-75% w/w cannabinoid; 15%-40% w/w eugenol; 5%-20% w/w menthol and/or menthone; and 0.1%-10% w/w d-limonene, the method comprising the steps of: a) providing a cannabinoid oil comprising the cannabinoid, heating said cannabinoid oil to at least about 60° C., and sonicating said cannabinoid oil to induce cavitation, wherein the temperature does not exceed about 80° C. during sonication; b) adding the eugenol to the sonicated cannabinoid oil of (a) and sonicating until the mixture is fully homogenized, wherein the temperature does not exceed about 60° C. during sonication; c) adding the d-limonene, menthol, and/or menthone to the mixture of (b) and sonicating until the mixture is fully homogenized, wherein the temperature does not exceed about 40° C. during sonication; d) sonicating the mixture of (c) until the solution has the desired particle size, wherein the temperature does not exceed about 55° C. during sonication, thereby preparing the therapeutic oil blend.

In one aspect, the present disclosure provides a method for preparing a sonicated, non-crystallizing therapeutic oil blend with emulsion-like properties, wherein the therapeutic oil blend comprises 30-90% w/w cannabidiol-related cannabinoid oil, 15-40% clove bud oil, and 5-20% peppermint oil, the method comprising the steps of: a) providing a fully liquified cannabidiol-related cannabinoid oil, a clove bud oil, and a peppermint oil; b) sonicating the fully liquified cannabidiol-related cannabinoid oil to induce cavitation, wherein the temperature of the cannabidiol-related cannabinoid oil does not exceed about 80° C. during sonication, thereby producing a sonicated cannabinoid oil; c) adding chilled clove bud oil to the sonicated cannabinoid oil and sonicating this mixture, thereby producing a sonicated eugenol mixture, wherein the temperature of the sonicated eugenol mixture does not exceed about 60° C. during sonication, and wherein the clove bud oil is added during a sonication step; d) adding chilled peppermint oil to the sonicated eugenol mixture and sonicating this mixture at low power, thereby producing a sonicated peppermint mixture, wherein the temperature of the sonicated peppermint mixture does not exceed about 40° C. during sonication, and wherein the peppermint oil is added during a sonication step; and e) further sonicating the sonicated peppermint mixture at high power to produce a sonicated therapeutic oil blend, wherein the temperature of the sonicated therapeutic oil blend does not exceed about 55° C. during sonication, thereby producing the sonicated therapeutic oil blend.

In one aspect, the present disclosure provides a method according to any one of any one of the foregoing embodiments, wherein the method produces a therapeutic oil blend according to any one of the foregoing embodiments.

### BRIEF DESCRIPTION OF THE SEVERAL VIEWS OF THE DRAWINGS

FIG. 1A-G show photos of a full spectrum hemp oil from different days of a crystallization study. FIG. 1A—day 1; FIG. 1B—day 7; FIG. 1C—day 14; FIG. 1D—day 15; FIG. 1E—day 16; FIG. 1F—day 21; and FIG. 1G—day 60.

FIG. 2A-F show photos of an exemplary therapeutic oil blend of the disclosure from different days of a crystallization study. FIG. 2A—day 1; FIG. 2B—day 7; FIG. 2C—day 14; FIG. 2D—day 21; FIG. 2E—day 60; and FIG. 2F—day 60.

FIG. 3A-G show images from different time points of a solvation study dissolving solid hemp oil in an MCT oil carrier. FIG. 3A—10 seconds after introduction of the solid hemp oil; FIG. 3B—60 seconds; FIG. 3C—120 seconds; FIG. 3D—180 seconds; FIG. 3E—240 seconds; FIG. 3F—300 seconds; and FIG. 3G—334 seconds.

FIG. 4A-F show images from different time points of a solvation study dissolving liquid hemp oil in an MCT oil carrier. FIG. 4A—10 seconds after introduction of the liquid hemp oil; FIG. 4B—30 seconds; FIG. 4C—45 seconds; FIG. 4D—60 seconds; FIG. 4E—75 seconds; and FIG. 4F—90 seconds.

FIG. 5A-F show images from different time points of a solvation study dissolving an exemplary therapeutic oil blend of the disclosure in an MCT oil carrier. FIG. 5A—1 second after introduction of the therapeutic oil blend; FIG. 5B—3 seconds; FIG. 5C—5 seconds; FIG. 5D—10 seconds; FIG. 5E—15 seconds; and FIG. 5F—20 seconds.

FIG. 6 shows a bar graph comparing the amount of time required by solid hemp oil, liquid hemp oil, and an exemplary therapeutic oil blend of the disclosure to dissolve into heated MCT oil.

FIG. 7 shows the results of an in vivo absorption assay for six different hemp-derived samples.

FIG. 8A-F shows photos documenting the physical state of six different hemp-derived samples tested in an in vivo absorption assay. FIG. 8A—Sample 1, traditional hemp oil; FIG. 8B—Sample 2, sonicated hemp oil; FIG. 8C—Sample 3, CBD in MCT; FIG. 8D—Sample 4, hemp and terpenes; FIG. 8E—Sample 5, CBD an terpenes in MCT; and FIG. 8F—Sample 6 exemplary therapeutic oil blend of the present disclosure.

FIG. 9A-C show thermal images of an area of skin pre- and post-topical application of an MCT oil carrier comprising CBD isolate. FIG. 9A—prior to application; FIG. 9B—30 seconds after application; FIG. 9C—60 seconds after application.

FIG. 10A-C show thermal images of an area of skin pre- and post-topical application of an exemplary therapeutic oil blend of the disclosure. FIG. 10A—prior to application; FIG. 10B—30 seconds after application; FIG. 10C—60 seconds after application.

FIG. 11 shows a line graph comparing the thermographic results over time of topical application of CBD in MCT oil versus a therapeutic oil blend of the disclosure.

FIG. 12A-C show microscope images of a crystallizing hemp oil at different magnification levels. FIG. 12A—4×; FIG. 12B—10×; FIG. 12C—20×.

FIG. 13A-C show microscope images of an exemplary therapeutic oil blend of the present disclosure at different magnification levels. FIG. 13A—4×; FIG. 13B—10×; FIG. 13C—20×.

FIG. 14 shows a comparison between a control full spectrum CBD-enriched hemp oil, maintained at room temperature over the course of 12 days (left) and a temperature-abused hemp oil, which experienced three freeze thaw cycles over the course of 12 days (right).

FIG. 15 shows a comparison between a control full spectrum CBD-enriched hemp oil formulated into an exemplary therapeutic oil blend of the disclosure, maintained at room temperature over the course of 12 days (left) and a temperature-abused therapeutic oil blend which experienced three freeze thaw cycles over the course of 12 days (right).

### DETAILED DESCRIPTION

### Definitions

The terminology used herein is for the purpose of describing particular embodiments only and is not intended to be limiting of the invention. As used herein, the singular forms “a,” “an” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner, similar to the term “comprising.” Therefore, the verb “comprise,” as is used in this description and in the claims and its conjugations, is used in its non-limiting sense to mean that items following the word are included, but items not specifically mentioned are not excluded.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

Throughout this application, the term “about” is used to indicate that a value includes the inherent variation of error for the device or the method being employed to determine the value, or the variation that exists among the samples being measured. Unless otherwise stated or otherwise evident from the context, the term “about” means within 10% above or below the reported numerical value (except where such number would exceed 100% of a possible value or go below 0%). When used in conjunction with a range or series of values, the term “about” applies to the endpoints of the range or each of the values enumerated in the series, unless otherwise indicated. As used in this application, the terms “about” and “approximately” are used as equivalents.

An “analgesic,” as used herein, refers to a substance that induces analgesia, or relief from pain. Analgesics act in various ways on the peripheral and central nervous systems to prevent, block, reduce, or eliminate the sensation of pain. As used herein, an “anesthetic” refers to a substance that induces anesthesia, or the temporary loss of sensation or awareness. Anesthetics temporarily affect, and in some instances completely eliminate, sensation, not exclusively limited to the sensation of pain. In some embodiments, a composition has improved analgesic properties compared to another composition with the same active ingredients due to increased bioavailability of the active ingredient.

As used herein, the term “bioavailability” refers to the proportion of a drug or other substance that is capable of being absorbed and used by the body, and is therefore able to have an active effect. In some embodiments improved bioavailability can be inferred by increased absorption into the body. In some embodiments, the therapeutic oil blends of the present disclosure exhibit increased skin absorbance compared to traditional hemp oils or to formulations that have not been mechanically agitated. In some embodiments, improved bioavailability can be inferred by improved medicinal effects. The therapeutic oil blends of the present disclosure also exhibit superior anesthetic effects compared to traditional hemp oil or formulations that have not been mechanically agitated.

As used herein, the term “cannabinoid” means an endocannabinoid receptor ligand. Cannabinoids include phytocannabinoids, which are cannabinoids that are naturally produced by plants of genus Cannabis, including the acidic and decarboxylated acid forms of the naturally-occurring plant-derived cannabinoids, and also includes cannabinoids produced from synthetic and biosynthetic methods that are identical to naturally-occurring plant-derived cannabinoids. Additional information about cannabinoids is provided in later sections of this application.

The term “cannabis” refers to a genus of flowering plants. Plants of genus cannabis include several species, including Cannabis sativa, Cannabis indica, and Cannabis ruderalis. There is a long history of cultivating plants of genus cannabis for hemp fibers, seeds and seed oils, medicinal purposes, and recreational activities.

The term “cannabis extract” as used herein refers to one or more plant extracts from the cannabis plant. A cannabis extract may contain, in addition to one or more cannabinoids, one or more non-cannabinoid components that are co-extracted with the cannabinoids from the plant material. Their respective ranges in weight will vary according to the starting plant material and the extraction methodology used. Cannabinoid-containing plant extracts may be obtained by various means of extraction of cannabis plant material. Such means include but are not limited to supercritical or subcritical extraction with CO₂, extraction with hot or cold gas and extraction with solvents.

As used herein, “cavitation” refers to the formation of an empty space within a substance, e.g., the formation of bubbles within a liquid. During sonication, cycles of pressure form thousands of microscopic vacuum bubbles in the solution. The bubbles collapse into the solution in a process known as cavitation.

The term “emulsifier” as used herein refers to amphiphilic molecules that are surface-active agents and that stabilize emulsions by reducing the interfacial tension. The term “surfactant” and “emulsifier” are used interchangeably.

The term “essential oils” as used herein refers to a concentrated hydrophobic liquid containing volatile aroma compounds. Essential oils may be plant-derived, synthetically produced, or biosynthetically produced. Essential oils are also known as volatile oils, ethereal oils, aetherolea, or the oil of the plant from which they were extracted. The term “essential oil” may also refer to natural plant oil typically obtained by distillation and having a chemical composition and organoleptic properties (e.g., fragrance) characteristic of the plant or other source from which it is extracted. As used herein, the term “fat” refers to saturated, mono-unsaturated and poly-unsaturated fatty acid. Fatty acids are usually present in the form of esters (e.g. mono-/di-/triglycerides). As used herein the term “oil” is used as a generic term for lipids, fats, or any mixture thereof.

A “carrier oil” is an oil used to dilute, distribute, and generally carry another substance with which it is mixed. Common carrier oils include plant-derived oils, e.g., vegetable oils and nut oils, and MCT oil.

“Hemp oil” refers to a botanical extract comprised of extracted hemp flowers, e.g., CO₂-extracted with an ethanol co-solvent. Post refinement removes most lipids and waxes in the oil. The oil is high in CBD, CBG, CBC with other minor cannabinoids. Terpenes are less than 1% total make up. This is distinct from Hemp seed oil, which is oil extracted from hemp seeds. In contrast with hemp oil, hemp seed oil does not comprise high levels of cannabinoids.

The term “nano-penetrative” as used herein refers to a composition, e.g., a therapeutic oil blend, that has an average particle size of less than 0.1 microns, thereby increasing bioavailability through a dermal membrane or mucous membrane. In some embodiments, all of the constituents of the composition have an average particle size of less than 0.1 microns. In some embodiments, some key ingredients of the composition, e.g., the cannabinoids and/or the terpenes, are suspended or contained within phases having an average particle size of less than 0.1 microns.

The term “pharmaceutical composition” as used herein refers to a composition that is pharmaceutically acceptable. The term “pharmaceutically acceptable” as used herein refers to compounds, material, compositions and/or dosage forms, which are, within the scope of sound medical judgment suitable for contact with the tissues of mammals, especially humans, without excessive toxicity, irritation, allergic response and other problem complications commensurate with a reasonable benefit/risk ratio. The term “excipient” as used herein refers to a pharmaceutically acceptable ingredient, which is commonly used in the pharmaceutical technology for preparing a granulate, solid or liquid oral dosage formulation. The term “cosmetic composition” is intended to mean a substance or a preparation intended to be brought into contact with the various superficial parts of the body, in particular the epidermis, the body-hair and head-hair systems, the nails, the lips and the oral mucous membranes.

As used within the context of this application, the term “purified” means extracted, isolated, and/or separated from other compounds, formulations, compositions, matter, and/or mass. In some embodiment, the term “purified” refers to a cannabinoid that is separated from other materials. In some embodiments, the term “purified” refers to a terpene that is separated from other materials. In some embodiments, purified means “substantially free” from other material, e.g., compounds, particles, vegetative material, plant derived substances, solvents, etc.

A “surfactant” as used herein refers to a substance that reduces the surface tension of a liquid in which it is dissolved.

The term “terpene” as used herein also covers terpenoids. Terpenes are lipophilic compounds, volatile and liquid at room temperature and are used herein in this invention as cannabinoids solubilizing agents. Terpenes are major secondary metabolites of cannabis and are responsible for the odor and flavor of various cannabis strains. Cannabis strains and hemp strains produce many terpenes as secondary metabolites. Terpenes are synthetized from terpene unit into mono-terpenes, sesqui-terpenes, di-terpenes that are lipophilic, volatile and insoluble in water and are cyclic or bicyclic or not cyclic and may have alcohol, aldehyde or ketone chemical moiety. The term “terpene” further relates to essential oils. The term “terpene” does not include fats and/or lipids.

“Treatment,” “treating,” “palliating” and “ameliorating,” as used herein, are used interchangeably. These terms refer to an approach for obtaining beneficial or desired results including but not limited to therapeutic benefit and/or a prophylactic benefit. Therapeutic benefit can be eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. Treating a disorder include reducing, alleviating, abating, ameliorating, relieving, or lessening a symptom associated with a disorder. In some embodiments, the term includes, but are not limited to, alleviation or amelioration of one or more symptoms or parameters associated with a disease, such as improvement in parameters as assessed various rating scales, tests or indices.

As used herein, the term “vasodilation” refers to the dilation of blood vessels. Vasodilation may be naturally or artificially induced, e.g., through the application of vasodilating compounds. Vasodilation may be accompanied by other physiological effects, such as lowering of blood pressure.

The term “vitamin E” refers to a group of compounds that include both tocopherols and tocotrienols including, but not limited to a-tocopherol, β-tocopherol, γ-tocopherol, δ-tocopherol, a-tocotrienol, β-tocotrienol, γ-tocotrienol, δ-tocotrienol, salts thereof, and combinations thereof. Vitamin E can be obtained from sources including, but not limited, to soybeans, sunflowers, and combinations thereof.

Embodiments of the present disclosure describe particle size. As used herein the term “particle size” refers to the largest diameter of particles within the referenced product. Thus, a rod-shaped crystal would have a particle size roughly equivalent to the length of the crystal. Particle size for a product (e.g., a cannabinoid formulation) refers to the mean particle size. That is, the particle sizes of all particles within the product. This can be calculated by measuring the particle size within a representative section of the product, by e.g., studying a sample of the product. In some embodiments, the particle size of a therapeutic oil blend of the present disclosure refers to the size of cannabinoid crystals within the therapeutic oil blend. Thus, in some embodiments, a particle size of less than 0.1 μm refers to a therapeutic oil blend that has an average CBD crystal size of less than 0.1 μm. In some embodiments, the particle size refers to the diameter of micelles or reverse micelles comprised by the therapeutic oil blend. Thus, in some embodiments, a particle size of less than 0.1 μm refers to a therapeutic oil blend that has an average reverse micelle size of less than 0.1 μm.

In some embodiments, “nano-penetrative,” as used herein, refers to a therapeutic oil blend with a particle size of less than about 0.1 μm. In some embodiments, “nano-penetrative” refers to a therapeutic oil blend that is able to penetrate the sensory nerve fibers via topical application. In some embodiments, the ability of a therapeutic oil blend to penetrate the sensory nerve fibers is demonstrated by a numbing of tactile or pain stimulus or sensatoin following topical application of the therapeutic oil blend.

As used herein, a “cannabidiol-related cannabinoid” refers to any one of cannabidiolic acid (CBDA), cannabidiol (CBD), cannabidiol monomethylether (CBDM), cannabidiol-C4 (CBD-C4), cannabidivarinic acid (CBDVA), cannabidivarin (CBDV), cannabidiorcol (CBD-C1), salts thereof, derivatives thereof or mixtures thereof. The present disclosure uses cannabinoid oils as ingredients for the therapeutic oil blend. In some embodiments, the cannabinoid oil comprises a cannabidiol-related cannabinoid.

As used herein, the term “shelf-stable” indicates that a cannabinoid product is capable of being stored for at least 30 days or at least 60 days at room temperature without crystallizing. Thus, in some embodiments, “shelf stable” therapeutic oil blends of the present disclosure stay liquid for at least 60 days at room temperature.

### Therapeutic Oil Blends

The present disclosure provides methods of formulating therapeutic oil blends comprising one or more cannabinoids and one or more terpenes and/or essential oils. In some embodiments, the therapeutic oil blend comprises cannabidiol (CBD). In some embodiments, the therapeutic oil blend comprises one or more terpenes and/or essential oils that provide additional desirable properties, e.g., improved shelf-stability or pharmaceutical effects such as analgesic, anesthetic, anti-inflammatory properties and the like. In some embodiments, methods of formulating the disclosed therapeutic oil blends result in nano-penetrative particle size, non-crystallizing properties, and/or improved homogeneity.

### Cannabis

Cannabis is a genus of flowering plants that includes three different species, Cannabis sativa, Cannabis indica and Cannabis ruderalis. There are 483 identifiable chemical constituents known to exist in the cannabis plant (Rudolf Brenneisen (2007) in Marijuana and the Cannabinoids, ElSohly, ed.; incorporated herein by reference), including at least 85 different cannabinoids and over 120 terpenes (El-Alfy, Abir T, et al. (2010) Pharmacology Biochemistry and Behavior 95 (4): 434-42; incorporated herein by reference). The two most well-known cannabinoids produced by Cannabis plants are tetrahydrocannabinol (THC) and cannabidiol (CBD).

### Cannabinoids

A cannabinoid is one of a class of diverse chemical compounds that acts on cannabinoid receptors such as CB1 and CB2. Ligands for these receptor proteins include the endocannabinoids (produced naturally in the body by animals), the phytocannabinoids (found in cannabis and some other plants), and synthetic cannabinoids (manufactured artificially).

Typical cannabinoids include, but are not limited to, Cannabigerolic Acid (CBGA), Cannabigerolic Acid monomethylether (CBGAM), Cannabigerol (CBG), Cannabigerol monomethylether (CBGM), Cannabigerovarinic Acid (CBGVA), Cannabigerovarin (CBGV), Cannabichromenic Acid (CBCA), Cannabichromene (CBC), Cannabichromevarinic Acid (CBCVA), Cannabichromevarin (CBCV), Cannabidiolic Acid (CBDA), Cannabidiol (CBD), Cannabidiol monomethylether (CBDM), Cannabidiol-C4 (CBD-C4), Cannabidivarinic Acid (CBDVA), Cannabidivarin (CBDV), Cannabidiorcol (CBD-C1), Tetrahydrocannabinolic acid A (THCA-A), Tetrahydrocannabinolic acid B (THCA-B), Tetrahydrocannabinolic Acid (THCA), Tetrahydrocannabinol (THC), Tetrahydrocannabinolic acid C4 (THCA-C4), Tetrahydrocannbinol C4(THC-C4), Tetrahydrocannabivarinic acid (THCVA), Tetrahydrocannabivarin (THCV), Tetrahydrocannabiorcolic acid (THCA-C4), Tetrahydrocannabiorcol (THC-C1), Δ7-cis-iso-tetrahydrocannabivarin, Δ8-tetrahydrocannabinolic acid (Δ8-THCA), Cannabivarinodiolic (CBNDVA), Cannabivarinodiol (CBNDV), Δ8-tetrahydrocannabinol (Δ8-THC), Δ9-tetrahydrocannabinol (Δ9-THC), Cannabicyclolic acid (CBLA), Cannabicyclol (CBL), Cannabicyclovarin (CBLV), Cannabielsoic acid A (CBEA-A), Cannabielsoic acid B (CBEA-B), Cannabielsoin (CBE), Cannabivarinselsoin (CBEV), Cannabivarinselsoinic Acid (CBEVA), Cannabielsoic Acid (CBEA), Cannabielvarinsoin (CBLV), Cannabielvarinsoinic Acid (CBLVA), Cannabinolic acid (CBNA), Cannabinol (CBN), Cannabivarinic Acid (CBNVA), Cannabinol methylether (CBNM), Cannabinol-C4 (CBN-C4), Cannabivarin (CBV), Cannabino-C2 (CBN-C2), Cannabiorcol (CBN-C1), Cannabinodiol (CBND), Cannabinodiolic Acid (CBNDA), Cannabinodivarin (CBDV), Cannabitriol (CBT), 10-Ethoxy-9-hydroxy-Δ6a-tetrahydrocannabinol, 8,9-Dihydroxy-Δ6a(10a)-tetrahydrocannabinol (8,9-Di-OH-CB T-C5), Cannabitriolvarin (CBTV), Ethoxy-cannabitriolvarin (CBTVE), Dehydrocannabifuran (DCBF), Cannbifuran (CBF), Cannabichromanon (CBCN), Cannabicitran (CBT), 10-Oxo-A6a(10a)-tetrahydrocannabinol (OTHC), Δ9-cis-tetrahydrocannabinol (cis-THC), Cannabiripsol (CBR), 3,4,5,6-tetrahydro-7-hydroxy-alpha-alpha-2-trimethyl-9-n-propyl-2,6-methano-2H-1-benzoxocin-5-methanol (OH-iso-HHCV), Trihydroxy-delta-9-tetrahydrocannabinol (triOH-THC), Yangonin, Epigallocatechin gallate, Dodeca-2E, 4E, 8Z, 10Z-tetraenoic acid isobutylamide, and Dodeca-2E,4E-dienoic acid isobutylamide. See Holley et al. (1975) J. Pharm. Sci. 64:892-894 and De Zeeuw et al. (9172) Science 175:778-779, each of which is herein incorporated by reference in its entirety for all purposes.

CBD is one of the active cannabinoids identified in cannabis. CBD does not appear to have any intoxicating effects such as those caused by THC in marijuana, but may have effects on anxiety, depression, psychological disorders, pain and post-traumatic stress disorder (PTSD), among other indications.

In some embodiments, the composition of the present disclosure comprises CBD. The terms “cannabidiol” and “CBD” are interchangeably used herein and refer to a non-psychotropic cannabinoid having structure as described in the formula below, salt or derivatives thereof, such as Δ⁴-cannabidiol, Δ⁵-cannabidiol, Δ⁶-cannabidiol, Δ^{1,7}-cannabidiol, Δ¹-cannabidiol, Δ²-cannabidiol, Δ³-cannabidiol.

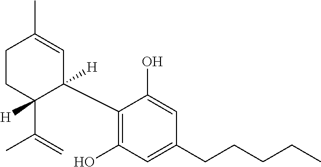 [SMILES: C=C(C)[C@@H]1CCC(C)=C[C@H]1c1c(O)cc(CCCCC)cc1O]

As used herein, the term “CBDA” refers to cannabidiolic acid and has the following structural formula:

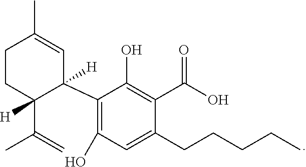 [SMILES: C=C(C)[C@@H]1CCC(C)=C[C@H]1c1c(O)cc(CCCCC)c(C(=O)O)c1O]

Decarboxylating CBDA with heat, light, etc., forms CBD and other possible cannabinoid derivatives.

In some embodiments the present disclosure teaches the use of Cannabidiol-related cannabinoids. In some embodiments, CBDV is a cannabidiol-related cannabinoid. As used herein, the term “CBDV” refers to cannabidivarin and has the following structural formula:

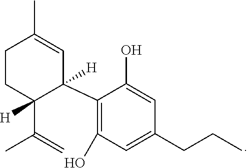 [SMILES: C=C(C)[C@@H]1CCC(C)=C[C@H]1c1c(O)cc(CCC)cc1O]

In some embodiments, CBDVA is a cannabidiol-related cannabinoid. As used herein, the term “CBDVA” refers to cannabidivarinic acid and has the following structural formula:

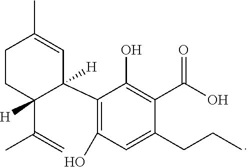 [SMILES: C=C(C)[C@@H]1CCC(C)=C[C@H]1c1c(O)cc(CCC)c(C(=O)O)c1O]

Decarboxylating CBDVA with heat, light, etc., forms CBDV and other possible cannabinoid derivatives.

In some embodiments, the composition comprises a cannabinoid selected from the group consisting of Cannabidiolic Acid (CBDA), Cannabidiol (CBD), Cannabidiol monomethylether (CBDM), Cannabidiol-C4 (CBD-C4), Cannabidivarinic Acid (CBDVA), Cannabidivarin (CBDV), Cannabidiorcol (CBD-C1), salts thereof, derivatives thereof and mixtures thereof. Each possibility represents a separate embodiment of the disclosure. In some embodiments, the composition comprises Cannabidiolic Acid (CBDA). In some embodiments, the composition comprises Cannabidiol (CBD). In some embodiments, the composition comprises Cannabidiol monomethylether (CBDM). In some embodiments, the composition comprises Cannabidiol-C4 (CBD-C4). In some embodiments, the composition comprises Cannabidivarinic Acid (CBDVA). In some embodiments, the composition comprises Cannabidivarin (CBDV). In some embodiments, the composition comprises Cannabidiorcol (CBD-C1).

In some embodiments, the cannabinoid is a phytocannabinoid. In some embodiments, the cannabinoid is a phytocannabinoid found in a Cannabis (or other) plant. In some embodiments, the cannabinoid is a synthetic cannabinoid. In some embodiments, the cannabinoid is a mixture of phytocannabinoids. In some embodiments, the cannabinoid is a mixture of synthetic cannabinoids. In some embodiments, the cannabinoid is a mixture of phyto and synthetic cannabinoids.

For the purposes of this disclosure, the term “phytocannabinoid” as used herein generally refers to a cannabinoid that can be found in, isolated from and/or extracted from a natural resource, regardless of how the product is actually produced. “Synthetic cannabinoids” are a class of chemicals that are different from the cannabinoids found e.g. in cannabis but which also bind to cannabinoid receptors.

The term “natural cannabinoid” as used herein generally refers to a cannabinoid that can be found in, isolated from and/or extracted from a natural resource, such as plants. “Synthetic cannabinoids” are a class of chemicals that are different from the cannabinoids found e.g. in cannabis but which also bind to cannabinoid receptors.

Most cannabinoids exist in two forms, as acids and in neutral (decarboxylated) forms. The acid form is designated by an “A” at the end of its acronym (i.e. THCA). The phytocannabinoids are synthesized in the plant as acid forms, and while some decarboxylation does occur in the plant, it increases significantly post-harvest and the kinetics increase at high temperatures. (Sanchez and Verpoorte 2008). Cannabinoids in their acid forms (those ending in “−A”) can be converted to their non-acidic forms through a process called decarboxylation. While some decarboxylation (e.g., neutralization) of cannabinoids does occur in the plant, production of the neutral forms increase significantly post-harvest. (Sanchez and Verpoorte (2008) Plant Cell Physiol. December: 49(12)). Full decarboxylation of phytocannabinoids can be catalyzed by post-cultivation heating cannabis plant material or extracted cannabinoids (e.g., by combustion, vaporization, or baking in an oven).

Within the context of this application, where reference is made to a particular cannabinoid, each of the acid and/or decarboxylated forms are contemplated as both single molecules and mixtures.

In order to find the total amount of cannabinoids in a sample (e.g., total amount of active non-acidic cannabinoid), the total measured content of acid cannabinoid variants forms should be adjusted to account for the loss of the carboxyl group. In some embodiments, this adjustment can be made by multiplying the molar content of the acidic cannabinoid forms by the molecular weight of the corresponding decarboxylated cannabinoid. Other shorthand conversions are also available for quickly converting acidic cannabinoid content to active cannabinoid content.

For example, in some embodiments, THCA can be converted to active THC using the formula: THCA×0.877=THC. When using this approach, the maximum THC for the sample is: THCmax=(THCA×0.877)+THC. This method has been validated according to the principles of the International Conference on Harmonization. Similarly, CBDA can be converted to active CBD and the yield is determined using the yield formula: CBDA×0.877=CBD. Also, the maximum amount of CBD yielded, i.e. max CBD for the sample is: CBDmax=(CBDA×0.877)+CBD. Additionally, CBGA can be converted to active CBG by multiplying CBGA by 0.878 (CBGmax=(CBGA×0.878)+CBG).

Cannabinoids and terpenes from any source may be used in the compositions of the present disclosure. In some embodiments, cannabinoid and or terpenes are extracted from Cannabis plants. In some embodiments, the cannabinoids and terpenes described herein are provided as Cannabis extracts (also interchangeably referred to herein as “Cannabis plant extracts.”) In some embodiments, the cannabinoid and/or terpene extracts are produced from aerial parts of Cannabis plants, e.g., the stalks, stems, leaves, and seeds.

Cannabis plant extracts can be produced according to methods known in the art. For example, suitable extraction methods include maceration, percolation, solvent extraction, steam distillation (giving you essential oil) or vaporization. General protocols for the preparation of Cannabis extracts from cannabis plant material are described in U.S. Pat. Nos. 8,603,515 and 9,730,911, both incorporated by reference herein.

Solvent extraction may be carried out using essentially any solvent that dissolves cannabinoids/cannabinoid acids, such as for example C1 to C5 alcohols (e.g. ethanol, methanol), C4-C12 alkanes (e.g. hexane or butane), Norflurane (HFA134a), HFA227, and carbon dioxide. When solvents such as those listed above are used, the resultant primary extract typically contains non-specific lipid-soluble material or “ballast” e.g. waxes, wax esters and glycerides, unsaturated fatty acid residues, terpenes, carotenes, and flavonoids. The primary extract may be further purified for example by “winterization”, which involves chilling to −20° C. followed by filtration to remove waxy ballast, supercritical or subcritical extraction, vaporization, distillation, and chromatography.

Additional extraction techniques for cannabinoids, including vaporizer-based approaches, can be found in U.S. Pat. Nos. 7,700,368, 10,159,908, U.S. Pub. No. 2019/0151771, U.S. Pub. No. 2018/0078874, U.S. Pub. No. 2020/0080021, U.S. Pub. No. 2020/0048214, U.S. Pub. No. 2020/0048215, and U.S. patent Ser. No. 10/555,914, each of which is incorporated by reference herein in its entirety.

In some embodiments, terpenes are extracted from Cannabis using a vacuum-drying oven. Vacuum-drying ovens remove water, solvents, and terpenes from the Cannabis. The solution of water, solvents, and terpenes can be separated by filtration to purify terpenes.

In some embodiments, terpenes and/or cannabinoids are extracted from Cannabis using carbon dioxide. In some embodiments, the carbon dioxide is supercritical carbon dioxide (scCO₂). Carbon dioxide extraction may occur at very low temperatures, preventing compounds like terpenes and cannabinoids from degrading. U.S. Pat. No. 9,744,200 and International Application No. 2016/200438 describe carbon dioxide extraction processes and are each incorporated by reference herein in their entireties.

In some embodiments, the compositions of the present disclosure comprise one or more components are derived from sources other than the Cannabis plant (e.g., from other organisms, or chemically synthesized). For example, the compositions of the present disclosure can, in some embodiments, comprise cannabinoids and/or terpenes produced via standard chemical, biochemical, or biocatalytic methods. Persons having skill in the art will be familiar with various synthesis methods, including those of U.S. Pat. No. 9,359,625 and Taura et al. 1996, The Journal of Biological Chemistry, Vol. 271, No. 21, p. 17411-17416.

Additionally, cannabinoids and terpenes of the present disclosure can be commercially sourced. For example, CBD and THC can be purchased from Sigma-Aldrich Company Ltd, Fancy Road, Poole Dorset, BH12 4QH, or may be chemically synthesized. Beta-pinene and limonene can also be purchased from Sigma-Aldrich Company Ltd, Fancy Road, Poole Dorset, BH12 4QH.

Some cannabinoids do not accumulate at high levels in cannabis plant material. In some embodiments, these cannabinoids can be produced by chemical means. For example, in some embodiments, cannabinoids such as cannabinol are created from THC or CBD as described in Pollastro et al. and Adams et al. which are each incorporated by reference herein in their entirety: Pollastro et al. J. Nat. Prod. 2018, 81, 3, 630-633; Adams et al. J. Am. Chem. Soc. 1940, 62, 9, 2402-2405.

Cannabinoids used in compositions and methods of the present disclosure can be derived from various sources, including but not limited to hemp (e.g., hemp stalk, hemp stem,), cannabis (e.g., cannabis flower, cannabis leaf, cannabis stalk, cannabis stem,), Echinacea purpurea, Echinacea angustifolia, Echinacea pallida, Acmella oleracea, Helichrysum umbraculigerum, Radula marginata, kava, black truffle, Syzygium aromaticum (cloves), Rosmarinus oficinalis, basil, oregano, black pepper, lavender, true cinnamon, malabathrum, cananga odorata, copaifera spp., and hops.

In some embodiments, the cannabinoids and terpenes of the present disclosure are pure isolates. In some embodiments, cannabinoids and/or terpenes are provided as a complex mixture (e.g., within a complex extract). A complex mixture contains two or more cannabinoids and/or terpenes. For example, in some embodiments, CBD and THC are provided as a solution containing 50% CBD and 50% THC.

In some embodiments, the composition is a topical formulation comprising the therapeutic oil blend of the disclosure. In some embodiments, the composition comprises additional cannabinoids. In some embodiments, the composition comprises additional terpenes.

In some embodiments, the therapeutic oil blend comprises cannabinoids in a weight percentage of about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90% or 95% by weight of the therapeutic oil blend, or any ranges or subranges therebetween. In some embodiments, the therapeutic oil blend comprises about 20% to about 80% by weight of a cannabinoid or a mixture of cannabinoids. In some embodiments, the composition comprises about 30% to about 70% by weight of a cannabinoid or a mixture of cannabinoids. In some embodiments, the therapeutic oil blend comprises a cannabis-derived composition, such as a hemp oil, comprising one or more cannabinoids.

In some embodiments, the therapeutic oil blend comprises CBD in a weight percentage of about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90% or 95% by weight of the therapeutic oil blend, or any ranges or subranges therebetween.

In some embodiments, the therapeutic oil blends of the present disclosure are produced using a cannabinoid oil. In some embodiments the cannabinoid oil is a hemp oil. As used herein, the term “hemp oil” refers to any cannabinoid extract in which CBD is the most abundant cannabinoid. In some embodiments the hemp oil comprises less than 0.3% THC w/w. In some embodiments, the hemp oil of the present disclosure comprises at least 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% CBD w/w. In some embodiments the hemp oil is full spectrum hemp oil. In some embodiments full spectrum hemp oil denotes that the hemp oil comprises one or more additional non-cannabinoid ingredients, such as terpenes present in the plant from which it was extracted. In some embodiments, full spectrum hemp oils are extracted with alcohol. Hemp oils are also often extracted with CO₂ or other hydrocarbons.

Persons having skill in the art are aware that high concentration cannabinoid oils have a tendency to crystallize at room temperature or colder. That is because most extraction methods pull cannabinoids into extracts that are beyond the supersaturation points for that cannabinoid. CBD in particular is known to “crash” out of solution, making it especially difficult to formulate with it. In order to overcome this problem, traditional formulators have turned to complex emulsions that incorporate surfactants that may not be natural, and that can often impart bad tastes/smells to the final product. As described in more detail herein, the present disclosure teaches methods of stabilizing cannabinoids, including CBD, so that they do not fall out of solution.

Thus, in some embodiments, the therapeutic oil blends of the present disclosure are non-crystallizing. As used herein, the term “non-crystallizing” refers to a cannabinoid composition that remains shelf stable, and does not crystallize for at least 60 days at room temperature. In some embodiments, the term “non-crystallizing” is applied to shelf stable compositions that nonetheless have at least one cannabinoid beyond its supersaturation point. That is, the solutions, in the absence of the additional ingredients or processing described herein, would begin to crystallize out of solution.

### Terpenes

Terpenes are a large and diverse class of organic compounds, produced by a variety of plants, particularly conifers and citrus plants, and by some insects such as termites or swallowtail butterflies, which emit terpenes from their osmeteria. They often have a strong odor and may protect the plants that produce them by deterring herbivores and by attracting predators and parasites of herbivores.

When terpenes are modified chemically, such as by oxidation or rearrangement of the carbon skeleton, the resulting compounds are generally referred to as terpenoids. The difference between terpenes and terpenoids is that terpenes are hydrocarbons, whereas terpenoids contain additional functional groups. As used herein, the term “terpene” encompasses terpenoids. Terpenoids are also known as isoprenoids. Any terpene can be converted to a terpenoid, synthetic terpenoid or semisynthetic terpenoid by an array of known chemical reactions. These conversions have been taught exhaustively in the art.

Terpenes and terpenoids are the primary constituents of the essential oils of many types of plants and flowers. Essential oils are used widely as fragrances in perfumery, and in medicine and alternative medicines such as aromatherapy. Synthetic variations and derivatives of natural terpenes and terpenoids also greatly expand the variety of aromas used in perfumery and flavors used in food additives. Terpenes are a major constituent of Cannabis sativa plants, which contain at least 120 identified compounds.

Chemically, as used herein, the term “terpene” refers to a compound built on an isoprenoid structure or produced by combining isoprene units, 5 carbon structures. Within the context of this disclosure, the term “terpene” does not necessarily require 5 carbons or multiples of 5 carbons. It is understood that a reaction with isoprene units does not always result in a terpene comprising all the carbon atoms. Within the context of this disclosure, the term “terpene” includes cannabis-derived terpenes and non-cannabis-derived terpenes. Within the context of this disclosure, the term “terpene” includes Hemiterpenes, Monoterpenols, Terpene esters, Diterpenes, Monoterpenes, Polyterpenes, Tetraterpenes, Terpenoid oxides, Sesterterpenes, Sesquiterpenes, Norisoprenoids, or their derivatives. As well as isomeric, enantiomeric, or optically active derivatives. Within the context of this disclosure, the term terpene includes the α-(alpha), β-(beta), γ-(gamma), oxo-, isomers, or any combinations thereof. Terpenes can be acyclic, monocyclic, or polycyclic. Derivatives of terpenes include terpenoids, hemiterpenoids, monoterpenoids, sesquiterpenoids, sesterterpenoid, sesquarterpenoids, tetraterpenoids, triterpenoids, tetraterpenoids, polyterpenoids, isoprenoids, and steroids.

Examples of terpenes within the context of this disclosure include, without limitation: 7,8-dihydro-alpha-ionone, 7,8-dihydro-beta-ionone, acetanisole, acetic acid, acetyl cedrene, anethole, anisole, benzaldehyde, bergamotene (alpha-cis-bergamotene) (alpha-trans-bergamotene), bisabolol (beta-bisabolol), alpha bisabolol, borneol, bornyl acetate, butanoic/butyric acid, cadinene (alpha-cadinene) (gamma-cadinene), cafestol, caffeic acid, camphene, camphor, capsaicin, carene (delta-3-carene), carotene, carvacrol, dextro-carvone, laevo-carvone, alpha-caryophyllene, beta-caryophyllene, caryophyllene oxide, cedrene (alpha-cedrene) (beta-cedrene), cedrene epoxide (alpha-cedrene epoxide), cedrol, cembrene, chlorogenic acid, cinnamaldehyde, alpha-amyl-cinnamaldehyde, alpha-hexyl-cinnamaldehyde, cinnamic acid, cinnamyl alcohol, citronellal, citronellol, cryptone, curcumene (alpha-curcumene) (gamma-curcumene), decanal, dehydrovomifoliol, diallyl disulfide, dihydroactinidiolide, dimethyl disulfide, eicosane/lcosane, elemene (beta-elemene), estragole, ethyl acetate, ethyl cinnamate, ethyl maltol, eucalyptol/1,8-cineole, eudesmol (alpha-eudesmol) (beta-eudesmol) (gamma-eudesmol), eugenol, euphol, farnesene, farnesol, fenchol (beta-fenchol), fenchone, geraniol, geranyl acetate, germacrenes, germacrene b, guaia-1(10),11-diene, guaiacol, guaiene (alpha-guaiene), gurjunene (alpha-gurjunene), herniarin, hexanaldehyde, hexanoic acid, hexyl acetate humulene (alpha-humulene) (beta-humulene), ionol (3-oxo-alpha-ionol) (beta-ionol), ionone (alpha-ionone) (beta-ionone), ipsdienol, isoamyl acetate, isoamyl alcohol, isoamyl formate, isoborneol, isomyrcenol, isopulegol, isovaleric acid, isoprene, kahweol, lavandulol, limonene, gamma-linolenic acid, linalool, longifolene, alpha-longipinene, lycopene, menthol, methyl butyrate, 3-mercapto-2-methylpentanal, mercaptan/thiols, beta-mercaptoethanol, mercaptoacetic acid, allyl mercaptan, benzyl mercaptan, butyl mercaptan, ethyl mercaptan, methyl mercaptan, furfuryl mercaptan, ethylene mercaptan, propyl mercaptan, thenyl mercaptan, methyl salicylate, methylbutenol, methyl-2-methylvalerate, methyl thiobutyrate, myrcene (beta-myrcene), gamma-muurolene, nepetalactone, nerol, nerolidol, neryl acetate, nonanaldehyde, nonanoic acid, ocimene, octanal, octanoic acid, p-cymene, pentyl butyrate, phellandrene, phenylacetaldehyde, phenylethanethiol, phenylacetic acid, pinene, alpha-pinene, beta-pinene, propanethiol, pristimerin, pulegone, phytolquercetin, retinol, rutin, sabinene, sabinene hydrate, cis-sabinene hydrate, trans-sabinene hydrate, safranal, alpha-selinene, alpha-sinensal, beta-sinensal, beta-sitosterol, squalene, taxadiene, terpin hydrate, terpineol, terpine-4-ol, alpha-terpinene, gamma-terpinene, terpinolene, thiophenol, thuj one, thymol, alpha-tocopherol, tonka undecanone, undecanal, valeraldehyde/pentanal, verdoxan, alpha-ylangene, umbelliferone, or vanillin. Additional examples of terpenes and their characteristics may be found in U.S. Publication No. 20170266153, herein incorporated by reference in its entirety.

Alpha-bisabolol refers to a compound with the following structural formula:

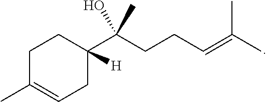 [SMILES: CC(C)=CCC[C@@](C)(O)[C@@H]1CC=C(C)CC1]

Alpha-bisabolol is often characterized as the active component of chamomile.

Beta-bisabolol refers to a compound with the following structural formula:

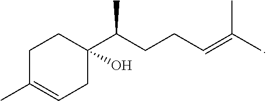 [SMILES: CC(C)=CCC[C@H](C)[C@@]1(O)CC=C(C)CC1]

Beta-bisabolol is often characterized as having a citrus, floral, lemon, sweet, herbaceous aroma.

As used herein, the term “borneol” refers to a compound having the following structural formula:

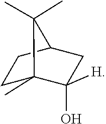

Borneol is often characterized as having a menthol aroma, similar to camphor. Borneol can also be converted into camphor.

As used herein, the term “beta-caryophyllene” refers to a compound with the following structural formula:

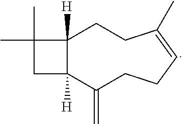

Beta-Caryophyllene is often characterized as a flavor component of black pepper. Beta-caryophyllene is often referred to as caryophyllene.

As used herein, the term “camphor” refers to a compound with either of the following structural formulas:

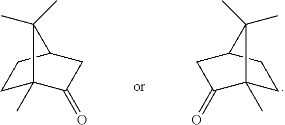

Within the context of this disclosure, the term “camphor” includes enantiomers as either a single form or a mixture in any ratio. Camphor is commonly found in varieties of Cinnamon bark, Rosemary, and Mint, and is a terpenoid with the chemical formula C₁₀H₁₆O. It may be used as an anti-inflammatory, analgesic, vasodilator, and aromatic agent. Camphor is readily absorbed through the skin stimulating the nerves for hot and cold. If heavy motion is applied to its application, it will give a warm sensation, a cooling one if applied softly. Stimulation of the nerves provides a local analgesic effect. Camphor has a very characteristic odor for which the tree is named. Camphor is used as a flavor and fragrance agent in chewing gum, hard candy, etc. Camphor can be used to improve skin healing (e.g., reconstructed human epidermis), as a local anesthetic, a muscle relaxant, an antipathogenic, and an antimicrobial agent.

As used herein, the term “citronellal” refers to a compound with the following structural formula:

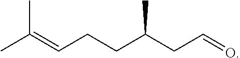

Citronellal is often characterized as making up to 80% of the leaf oil from Kaffir lime leaves and as the characteristic aroma.

As used herein, the term “eucalyptol” refers to a terpene with the following structural formula:

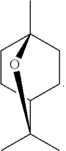

Found in Eucalyptus leaves, Eucalyptol makes up 90% of Eucalyptus Oil. Eucalyptol has many of the same properties and effects of Camphor and Menthol. It is used in mouthwashes, topicals, and medical preparations. When used as a cough suppressant, its anti-inflammatory properties are shown via cytokine inhibition. When applied topically, it reduces inflammation and pain. Eucalyptol is often characterized as having a mint-like smell. Eucalyptol is also known by 1,8-cineol, 1,8-cineole, cajeputol, 1,8-epoxy-p-menthane, 1,8-oxido-p-menthane, eucalyptol, eucalyptole, 1,3,3-trimethyl-2-oxabicyclo[2,2,2]octane, cineol, and cineole. Eucalyptol can be used as an antifungal agent, to alleviate inflammation (e.g., lung inflammation), an antioxidant, and an anticancer agent.

As used herein the term “eugenol” refers to a compound with the following structural formula:

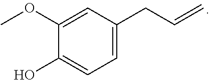

A terpene primarily found in cloves, cinnamon, and bay leaves, eugenol has long been used in dentistry for toothaches, mouthwashes, soaps, insect repellents, foods, and various veterinary medications. It is well documented in its anti-inflammatory, anesthetic, anti-microbial, and antioxidant properties. Eugenol also aids in penetrating into the dermis. The chemical formula is C₁₀H₁₂O₂. Eugenol is often characterized as causing the aromatic smell of cloves. Eugenol is found in insect attractants as well as UV absorbers. Eugenol is well known for its anesthetic, analgesic, anti-inflammatory, and surfactant properties. In some embodiments, it may be used as a penetrative agent to aid in absorption into the skin, enabling the delivery of active agents (e.g., cannabinoids and terpenes) into the dermis where the sensory nerve fibers and the cutaneous blood vessels reside in the reticular layer.

As used herein, the term “farnesol” refers to a compound with the following structural formula:

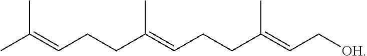 [SMILES: CC(C)=CCC/C(C)=C/CC/C(C)=C/CO]

Farnesol is often characterized as having a mild, fresh, sweet, floral, linden tree odor. Farnesol is used in cosmetics, flavors and fragrances.

As used herein, the term “geraniol” refers to a compound of the following structural formula:

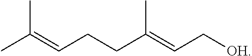

Geraniol is often characterized as having a sweet-rose like scent.

As used herein, the term “alpha-humulene” refers to a compound with the following structural formula:

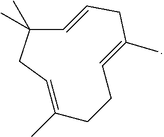

Alpha-humulene is often characterized as contributing to the flavor profile of beer. Alpha-humulene is sometimes often referred to as simply “Humulene” or “Caryophyllene” and in the context of this disclosure both may be used interchangeably.

As used herein, the term “beta-humulene” refers to a compound with the following structural formula:

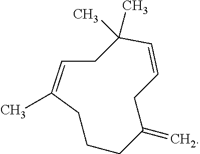

Beta-humulene is often characterized as having a green aroma.

As used herein, the term “isopulegol” refers to a compound with the following structural formula:

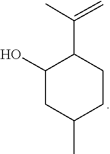

Isopulegol is often characterized as having a medium strength odor that is minty, cooling and bittersweet. Within the context of this disclosure, “isopulegol” may also refer to any number of isomeric forms.

As used herein, the term “limonene” refers to a terpene with the following structural formula:

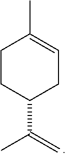

Limonene is often characterized as having a smell similar to oranges and other citrus fruits. It is commonly derived from citrus peels. Limonene is used in topicals as a penetrative agent with studies showing it can be applied topically to the skin and reach the mammary tissue with no toxicity. Limonene also has anti-inflammatory properties as well as antimicrobial properties. Within the context of this disclosure, the term “Limonene” encompasses all possible enantiomers and isomers of the compound in as either individual compounds or in a racemic mixture, e.g., d-Limonene. Limonene can be used to reduce anxiety and depression, to dissolve cholesterol-containing gallstones, to neutralize gastric acid, support normal peristalsis, relieve heartburn and gastroesophageal reflux, to improve immune function, and as a chemopreventative against cancer.

As used herein, the term “linalool” refers to a terpene with the following structural formula:

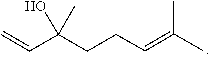

Linalool is found in lavender, mint, and laurels. Linalool possesses several pharmacological activities including anti-inflammatory, anxiolytic, anticonvulsant and antinociceptive. Linalool has two known enantiomeric forms. (S)-(+)-Linalool is often characterized as sweet and floral and the (R)-form is more woody and lavender-like. Within the context of this disclosure, the “Linalool” refers to either of the enantiomers or a racemic mixture of the two. Linalool can be used for reducing anxiety, reducing inflammation (e.g., lung inflammation), to improve Alzheimer's disease or symptoms thereof, as a sedative, an analgesic, an anti-microbial, an antibacterial, and an anti-epileptic.

As used herein, the term “menthol” refers to a compound with the following structural formula:

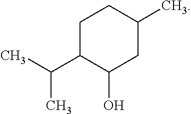

Menthol is often characterized as having a cooling, minty, peppermint aroma and flavor. Menthol is obtained from cornmint, peppermint or other mint varieties. It is a terpenoid with the chemical formula C₁₀H₂₀O. Used as an anti-inflammatory, analgesic, vasodilator, and aromatic agent. Menthol is readily absorbed through the skin stimulating the nerves for hot and cold. If heavy motion is applied to its application, it will give a warm sensation, a cooling one if applied softly. Menthol has a counterirritant effect on skin and mucous membranes producing anesthetic effects. Menthol is used as a penetrative aid in transdermal applications and may be used to promote vasodilation, anti-inflammation, and smell profile. Menthol can also be used to desensitize α3β4 nicotinic acetylcholine receptors.

Menthone: is a monoterpene with a minty flavor that occurs naturally in a number of essential oils. Menthone may be used to promote vasodilation, anti-inflammation, and smell profile.

Beta-Myrcene (or b-Myrcene) is a monoterpene that has the chemical formula C₁₀H₁₆. Myrcene is commonly produced from b-Pinene, however can be found in Wild Thyme, Hopps, Lemon Grass, and Cardamom. Myrcene has uses as an anti-inflammatory agent, anabolic agent, and a plant metabolite. Myrcene has sedative properties and is known to help cannabinoids cross the blood-brain barrier. Beta-Myrcene is often characterized as having an earthy, fruity clove-like odor. Beta-myrcene is also referred to as “myrcene” or “b-myrcene”. Myrcene can be used as an antibacterial, a neuroprotective agent, an antinociceptive, an analgesic, and to alleviate neuropathic pain, peptic ulcer disease, and inflammation. Depending on concentration, myrcene can be used as a sedative (e.g., over 0.5% myrcene) or to provide energizing effects (e.g., less than 0.5% myrcene). Beta-myrcene has the following structure:

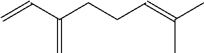

As used herein, the term “nerolidol” refers to a compound with either of the following structural formulas or any mixture thereby:

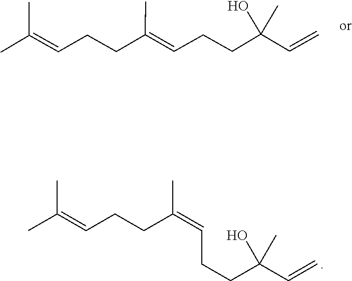

Nerolidol is often characterized as having a woody aroma, similar to fresh bark. There are two isomers of nerolidol, cis and trans, which differ in the geometry about the central double bond. Within the context of this disclosure, the term “Nerolidol” refers to either or both of the cis and trans isomers. Also known as peruviol and penetrol, Nerolidol is a naturally occurring sesquiterpene alcohol found in the essential oils of many types of plants and flowers. Nerolidol is has a wide range of beneficial effects, including antifungal and antibacterial effects. This terpene has a gentle scent and can also be found in ginger, jasmine, lemongrass and tea tree.

As used herein, the term “ocimene” refers to a compound with one of the isomeric forms:

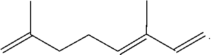

Within the context of this disclosure, ocimene refers to any of the isomers in a single pure form or a mixture in any ratio.

As used herein, the term “phytol” refers to a compound with the following structural formula:

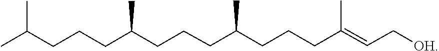 [SMILES: C/C(=C\CO)CCC[C@H](C)CCC[C@H](C)CCCC(C)C]

Phytol is often characterized having a mild floral, balsamic, and green tea type of aroma.

As used herein, the term “alpha-pinene” refers to a compound with either of the following structural formulas:

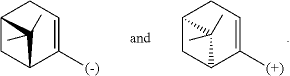

Alpha-Pinene has the chemical formula C₁₀H₁₆. Commonly found in Rosemary, Pine trees, and Eucalyptus, a-Pinene is highly bioavailable for 60% pulmonary uptake with rapid metabolism or redistribution. Its anti-inflammatory properties are expressed via PGE1. It is an acetylcholinesterase inhibitor which are a class of compounds known to aid in memory and increased alertness. Alpha-Pinene is often characterized as having a pine tree like aroma. Alpha-pinene can be used as an anti-inflammatory, an antiangiogenic, an anti-ulcer agent, and a bronchodilator. The terms a-pinene and alpha pinene are used interchangeably.

As used herein, the term “beta-pinene” refers to a terpene with the following structural formula:

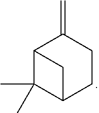

Beta-Pinene is often characterized as having a woody-green pine-like smell. Beta-Pinene is one of the most abundant compounds released by forest trees. Beta-Pinene is an isomer of pinene. Beta-Pinene is commonly found in Allspice and Nutmeg. Beta-Pinene is also used as an antimicrobial agent. The terms b-pinene and beta pinene are used interchangeably.

As used herein, the term “pulegone” refers to a compound with the following structural formula:

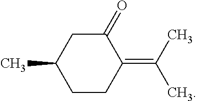

Pulegone is often characterized as having a smell similar to peppermint.

As used herein, the term “alpha-terpinene” refers a compound the following structural formula:

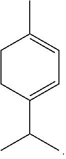

Alpha-terpinene is often characterized as having a lemony-citrus aroma. Alpha-terpinene is commonly derived from Allspice, cardamom, and marjoram oils. It may be used as a binding agent. It is one of the 3 isomeric hydrocarbons classified as monoterpenes from the synthesis of a-Pinene. Terpinene can be used as an antioxidant, an anti-inflammatory, an antimicrobial, an antiproliferative, to reduce oxidative stress, and to manage diabetes. The term a-terpinene and alpha terpinene are interchangeable.

As used herein, the term “gamma-terpinene” refers to a compound with the following structural formula:

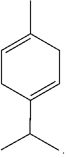

Gamma-terpinene is often characterized as having an herbaceous, citrusy sweet aroma. Gamma-terpinene is commonly derived from Allspice, cardamom, and marjoram oils. It may be used as a binding agent. It is one of the 3 isomeric hydrocarbons classified is monoterpenes from the synthesis of a-Pinene. Terpinene can be used as an antioxidant, an anti-inflammatory, an antimicrobial, an antiproliferative, to reduce oxidative stress, and to manage diabetes. The term y-terpinene and gamma terpinene are interchangeable.

As used herein, the term “terpinolene” or “delta-terpinene” refers to a compound with the following structural formula:

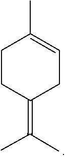

Terpinolene is often characterized as having an herbal aroma. Terpinolene's flavor has been described as sweet, woody, lemon, and lime-like.

As used herein, the term “thymol” refers to a compound with the following structural formula:

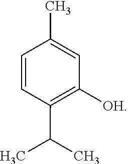

Thymol is often found in oil of thyme.

Terpenes in compositions of the present disclosure can be selected to provide benefits for particular conditions or subjects. In some embodiments, the terpene or the mixture of terpenes solubilize a cannabinoid or a mixture of cannabinoids. Each possibility represents a separate embodiment of the invention. In some embodiments, terpenes can be employed in combination with each other, as well as in combination with cannabinoids, to ameliorate one or more health conditions. For example, terpinolene, terpineol and linalool or lavender, valerian and jasmine essential oils can be combined with cannabinoids or cannabis extract to act as a sleep aid or treat sleep disorders. Beneficially, the combination of cannabinoids and terpenes, such as in a single composition, can have a synergistic effect on a subject's endocannabinoid system. For example, the presence of the terpenes can increase bioavailability of the cannabinoids. Alternatively or in addition, the presence of the cannabinoids can increase bioavailability of the terpenes.

In some embodiments, the therapeutic oil blend of the present disclosure comprises one or more terpenes selected from beta-myrcene, linalool, alpha-pinene, beta-pinene, beta-caryophyllene, caryophyllene oxide, camphor, alpha-humulene, nerolidol, d-limonene, 1-limonene, para-cymene, eugenol, farnesol, geraniol, phytol, menthol, menthone, terpineol, alpha-terpineol, benzaldehyde, hexyl acetate, methyl salicylate, eucalyptol, ocimene, terpinolene, alpha-terpinene, isopulegol, guaicol, alpha-bisabolol, or any combination thereof.

In some embodiments, the therapeutic oil blend comprises about 0.05% to about 80% by weight of a terpene or a mixture thereof. In some embodiments, the therapeutic oil blend comprises about 0.05%, 0.1%, 0.5%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, or 95% by weight of a terpene or a mixture thereof. In some embodiments, the therapeutic oil blend comprises about 0.1% to about 50% by weight of a terpene or a mixture thereof. In some embodiments, the pharmaceutical composition comprises about 5% to about 20% by weight of a terpene or a mixture of terpenes.

In some embodiments, the terpenes are provided as purified compounds. In other embodiments, the terpenes of the present disclosure are provided in the form of one or more essential oils. For example, in some embodiments, Eugenol is provided in the form of clove oil, which is greater than 80 percent eugenol. In some embodiments, menthol, menthone, d-limonene, camphor, eucalyptol, a-pinene, b-pinene, b-myrcene, a-terpinene, y-terpinene, and linalool are provided in the form of peppermint oil.

### Essential Oils

In some embodiments, the therapeutic oil blend of the present disclosure comprises essential oils.

The therapeutic oil blends of the present disclosure can comprise one or more essential oils or essential oil compounds. Essential oils can include, but are not limited to: Ylang Ylang (Cananga odorata); Yarrow (Achillea millefolium); Violet (Viola odorata); Vetiver (Vetiveria zizanoides); Vanilla (Vanilla plantifolia); Tuberose (Polianthes tuberosa); Thyme (Thymus vulgaris L.); Tea Tree (Melaleuca alternifolia); Tangerine (Citrus reticulata); Spruce, Black (Picea mariana); Spruce (Tsuga Canadensis); Spikenard (Nardostachys jatamansi); Spearmint (Mentha spicata); Sandalwood (Santalum spicatum); Rosewood (Aniba rosaeodora); Rosemary Verbenone (Rosmarinus officinalis); Rosemary (Rosmarinus officinalis); Rose (Rosa damascena); Rose Geranium (Pelargonium roseum); Ravensara (Ravensara aromatica); Plai (Zingiber cassumunar) Pine Needle (Pinus sylvestris L.); Petitgrain (Citrus aurantium); Peppermint (Mentha piperita); Pepper, Black (Piper nigrum L.); Patchouli (Pogostemon cablin); Palo Santo (Bursera graveolens); Palmarosa (Cymbopogon martini); Osmanthus (Osmanthus fragrans); Oregano (Origanum vulgare); Orange, Sweet (Citrus sinensis); Oak Moss (Evernia prunastri); Nutmeg (Myristica fragrans) Niaouli (Melaleuca viridifloria); Neroli (aka Orange Blossom) (Citrus aurantium); Myrtle (Myrtus communis); Myrrh (Commiphora myrrha); Mimosa (Acacia decurrens); Melissa (Melissa officinalis L.); Marjoram, Sweet (Origanum majorana); Manuka (Leptospermum scoparium); Mandarin, Red (Citrus deliciosa); Mandarin (Citrus deliciosa); Lotus, White (Nelumbo nucifera); Lotus, Pink (Nelumbo nucifera); Lotus, Blue (Nelumbo nucifera); Lime (Citrus aurantifolia); Lily (Lilum aurantum); Lemongrass (Cymbopogon citratus); Lemon (Citrus limonum); Lavender (Lavandula angustifolium); Lavandin (Lavandula hybrida grosso); Kanuka (Kunzea ericoides); Juniper Berry (Juniperus communis); Jasmine (Jasminum officinale); Jasmine Abs (Jasminum sambac); Helichrysum (Helichrysum italicum); Grapefruit, White (Citrus×paradisi); Grapefruit, Pink (Citrus paradisi); Ginger (Zingiber officinalis); Geranium (Pelargonium graveolens); Geranium, Bourbon (Pelargonium graveolens, ‘Herit); Gardenia (Gardenia jasminoides); Galbanum (Ferula galbaniflua); Frankincense (Boswellia carterii); Frangipani (Plumeria alba); Fir Needle White (Abies alba); Fir Needle Siberia (Abies siberica); Fir Needle Canada (Abies balsamea); Fennel, Sweet (Foeniculum vulgare); Eucalyptus Smithii. Eucalyptus Radiata, Eucalyptus Globulus, Eucalyptus Citriodora, Eucalyptus Blue Mallee (Eucalyptus polybractea); Elemi (Canarium luzonicum); Dill (Anethum graveolens); Cypress (Cupressus sempervirens); Cumin (Cuminum cyminum); Coriander (Coriandum sativum); Cocoa (Theobroma cacao); Clove (Eugenia caryophylatta); Clary Sage (Salvia sclarea); Cistus (aka Labdanum) (Cistus ladaniferus L.); Cinnamon (Cinnamomum zeylanicum); Chamomile, Roman (Anthemis nobilis); Chamomile, Blue (Matricaria chamomilla); Celery Seed (Apium graveolins); Cedarwood, Western Red (Thuja plicata); Cedarwood, Blood (Juniperus virginiana); Cedarwood Atlas (Cedrus atlantica); Carrot Seed (Daucus carota); Cardamon (Elettaria cardamomum); Caraway Seed (Carum carvi); Cajeput (Melaleuca cajuputi); Cade (Juniperus oxycedrus); Birch, White (Betula alba); Birch, Sweet (Betula lenta); Bergamot (Citrus bergamia); Bay Laurel (Laurus nobilis); Basil (Ocimum basilicum); Basil, Holy (Ocimum sanctum); Basil (Ocimum basilicum); Balsam Poplar (Populus balsamifera); Balsam Peru (Myroxylon balsamum); Angelica (Angelica archangelica L.); and/or combinations thereof.

In some embodiments, the composition comprising the therapeutic oil blend of the present disclosure may comprise an essential oil derived from a plant selected from the following list: Alfalfa (Medicago sativa L.); Allspice (Pimenta officinalis Lindl.); Almont, bitter (pure from prussic acid) (Prunnus amygdalus Batsch, Prussun armeniaca L., or Prunnus persica (L.) Batsch.); Ambrette (seed) (Hibiscus moschatus Moench.); Angelica root (Angelica archangelica L.); Angelica seed; Angelica stem; Angostura (cusparia bark) (Galipea officinalis Hancock.); Anise (Pimpinella anisum L.); Asafetida (Ferula assa-foetida L. and related spp. of Ferula.); Balm (lemon balm) (Melissa officinalis L.); Balsam of Peru (Myroxylon pereirae Klotzsch.); Basil (Ocimum basilicum L.); Bay leaves (Laurus nobilis L.); Bay (myrcia oil) (Pimenta racemosa (Mill.) J. W. Moore.); Bergamot (bergamot orange) (Citrus aurantium L. subsp. bergamia Wright et Am.); Bitter almond (free from prussic acid) (Prunus amygdalus Batsch, Prunus armeniaca L., or Prunus persica (L.) Batsch.); Bois de rose (Aniba rosaeodora Ducke.); Cacao (Theobroma cacao L.); Camomile (chamomile) flowers, Hungarian (Matricaria chamomilla L.); Camomile (chamomile) flowers, Roman or English (Anthemis nobilis L.); Cananga (Cananga odorata Hook. f and Thoms.); Capsicum (Capsicum frutescens L. and Capsicum annuum L.); Caraway (Carum carvi L.); Cardamom seed (cardamon) (Elettaria cardamomum Maton.); Carob bean (Ceratonia siliqua L.); Carrot (Daucus carota L.); Cascarilla bark (Croton eluteria Benn.); Cassia bark, Chinese (Cinnamomum cassia Blume.); Cassia bark, Padang or Batavia (Cinnamomum burmanni Blume.); Cassia bark, Saigon (Cinnamomum loureirii Nees.); Celery seed (Apium graveolens L.); Cherry, wild, bark (Prunus serotina Ehrh.); Chervil (Anthriscus cerefolium (L.) Hoffm.); Chicory (Cichorium intybus L.); Cinnamon bark, Ceylon (Cinnamomum zeylanicum Nees.); Cinnamon bark, Chinese (Cinnamomum cassia Blume.); Cinnamon bark, Saigon (Cinnamomum loureirii Nees.); Cinnamon leaf, Ceylon (Cinnamomum zeylanicum Nees.); Cinnamon leaf, Chinese (Cinnamomum cassia Blume.); Cinnamon leaf, Saigon (Cinnamomum loureirii Nees.); Citronella (Cymbopogon nardus Rendle.); Citrus peels (Citrus spp.); Clary (clary sage) (Salvia sclarea L.); Clover (Trifolium spp.); Coca (decocainized) (Erythroxylum coca Lam. and other spp. of Erythroxylum.); Coffee (Coffea spp.); Cola nut (Cola acuminata Schott and Endl., and other spp. of Cola.); Coriander (Coriandrum sativum L.); Cumin (cummin) (Cuminum cyminum L.); Curacao orange peel (orange, bitter peel) (Citrus aurantium L.); Cusparia bark (Galipea officinalis Hancock.); Dandelion (Taraxacum officinale Weber and T. laevigatum DC.); Dandelion root; Dog grass (quackgrass, triticum) (Agropyron repens (L.) Beauv.); Elder flowers (Sambucus canadensis L. and S. nigra I.); Estragole (esdragol, esdragon, tarragon) (Artemisia dracunculus L.); Estragon (tarragon); Fennel, sweet (Foeniculum vulgare Mill.); Fenugreek (Trigonella foenum-graecum L.); Galanga (galangal) (Alpinia officinarum Hance.); Geranium (Pelargonium spp.); Geranium, East Indian (Cymbopogon martini Stapf.); Geranium, rose (Pelargonium graveolens L′Her.); Ginger (Zingiber officinale Rosc.); Grapefruit (Citrus paradisi Macf.); Guava (Psidium spp.); Hickory bark (Carya spp.); Horehound (hoarhound) (Marrubium vulgare L.); Hops (Humulus lupulus L.); Horsemint (Monarda punctata L.); Hyssop (Hyssopus officinalis L.); Immortelle (Helichrysum augustifolium DC.); Jasmine (Jasminum officinale L. and other spp. of Jasminum.); Juniper (berries) (Juniperus communis L.); Kola nut (Cola acuminata Schott and Endl., and other spp. of Cola.); Laurel berries (Laurus nobilis L.); Laurel leaves (Laurus spp.); Lavender (Lavandula officinalis Chaix.); Lavender, spike (Lavandula latifolia Vill.); Lavandin (Hybrids between Lavandula officinalis Chaix and Lavandula latifolin Vill.); Lemon (Citrus limon (L.) Burm. f.); Lemon balm (see balm); Lemon grass (Cymbopogon citratus DC. and Cymbopogon lexuosus Stapf.); Lemon peel (Citrus limon (L.) Burm. f.); Lime (Citrus aurantifolia Swingle.); Linden flowers (Tilia spp.); Locust bean (Ceratonia siliqua L,); Lupulin (Humulus lupulus L.); Mace (Myristica fragrans Houtt.); Mandarin (Citrus reticulata Blanco.); Marjoram, sweet (Majorana hortensis Moench.); Mate (Ilex paraguariensis St. Hil.); Melissa (see balm); Menthol (Mentha spp.); Menthyl acetate; Molasses (extract) (Saccarum officinarum L.); Mustard (Brassica spp.); Naringin (Citrus paradisi Macf.); Neroli, bigarade (Citrus aurantium L.); Nutmeg (Myristica fragrans Houtt.); Onion (Allium cepa L.); Orange, bitter, flowers (Citrus aurantium L.); Orange, bitter, peel; Orange leaf (Citrus sinensis (L.) Osbeck.); Orange, sweet; Orange, sweet, flowers; Orange, sweet, peel; Origanum (Origanum spp.); Palmarosa (Cymbopogon martini Stapf.); Paprika (Capsicum annuum L.); Parsley (Petroselinum crispum (Mill.) Mansf.); Pepper, black (Piper nigrum L.); Pepper, white; Peppermint (Mentha piperita L.); Peruvian balsam (Myroxylon pereirae Klotzsch.); Petitgrain (Citrus aurantium L.); Petitgrain lemon (Citrus limon (L.) Burm. f.); Petitgrain mandarin or tangerine (Citrus reticulata Blanco.); Pimenta (Pimenta officinalis Lindl.); Pimenta leaf (Pimenta officinalis Lindl.); Pipsissewa leaves (Chimaphila umbellata Nutt.); Pomegranate (Punica granatum L.); Prickly ash bark (Xanthoxylum (or Zanthoxylum) Americanum Mill. or Xanthoxylum clava-herculis L.); Rose absolute (Rosa alba L., Rosa centifolia L., Rosa damascena Mill., Rosa gallica L., and vars. of these spp.); Rose (otto of roses, attar of roses); Rose buds; Rose flowers; Rose fruit (hips); Rose geranium (Pelargonium graveolens L′Her.); Rose leaves (Rosa spp.); Rosemary (Rosmarinus officinalis L.); Saffron (Crocus sativus L.); Sage (Salvia officinalis L.); Sage, Greek (Salvia triloba L.); Sage, Spanish (Salvia lavandulaefolia Vahl.); St. John's bread (Ceratonia siliqua L.); Savory, summer (Satureia hortensis L.); Savory, winter (Satureia montana L.); Schinus molle (Schinus molle L.); Sloe berries (blackthorn berries) (Prunus spinosa L.); Spearmint (Mentha spicata L.); Spike lavender (Lavandula latifolia Vill.); Tamarind (Tamarindus indica L.); Tangerine (Citrus reticulata Blanco.); Tarragon (Artemisia dracunculus L.); Tea (Thea sinensis L.); Thyme (Thymus vulgaris L. and Thymus zygis var. gracilis Boiss.); Thyme, white; Thyme, wild or creeping (Thymus serpyllum L.); Triticum (see dog grass); Tuberose (Polianthes tuberosa L.); Turmeric (Curcuma longa L.); Vanilla (Vanilla planifolia Andr. or Vanilla tahitensis J. W. Moore.); Violet flowers (Viola odorata L.); Violet leaves; Violet leaves absolute; Wild cherry bark (Prunus serotina Ehrh.); Ylang-ylang (Cananga odorata Hook. f. and Thoms.); and Zedoary bark (Curcuma zedoaria Rosc.).

Persons having skill in the art will be familiar with sources for various essential oils. Indeed recited essential oils are commercially available under a variety of brand names, including those from Healing Solutions®, Gya Labs® and Ethereal Nature. As used herein the term “clove bud oil” or “clove bud essential oil” refers to an oil comprising between 50-99% eugenol. In some embodiments, clove bud essential oil comprises greater than 90%, 91%, 92%, 93%, 94%, 95%, 96%, 97%, 98%, or 99% eugenol. As used herein, the term “peppermint oil” or peppermint essential oil” refers to an oil comprising menthol, and menthone. In some embodiments, peppermint essential oil may also comprise one or more terpenes selected from the group consisting of camphor, eucalyptol, a-pinene, b-pinene, b-myrcene, a-terpinene, d-limonene, y-terpinene, and linalool. In some embodiments, the peppermint oil comprises, about 1% camphor, about 46% menthol, about 40% menthone, about 1.56% a-pinene, about 1.62% b-pinene, about 4% b-myrcene, and about 13% d-limonene.

### Physical Characteristics of Therapeutic Oil Blend

The present disclosure provides novel therapeutic oil blends, and methods of formulation thereof, that overcome common problems prevalent among typical cannabinoid oil formulations. Existing formulations often encounter problems of large particle size, low absorption, short shelf life, and poor stability. For example, many existing formulations encounter difficulties with separation of constituent ingredients and/or crystallization over time, often on short time scales. This necessitates re-homogenization prior to use, a time-consuming and inconvenient process limiting their use on demand. Furthermore, these problems with existing oil formulations result in their limited ability to be mixed into other compositions and commercial products.

By contrast, the present therapeutic oil blends may have numerous beneficial physical characteristics differentiating them from currently available formulations. These properties may include improved shelf stability, which characteristic includes both non-separation and non-crystallization under typical storage conditions. In some embodiments, the therapeutic oil blend has improved shelf stability compared to existing therapeutic oil blends. In some embodiments, the therapeutic oil blend is liquid at room temperature. In some embodiments, the therapeutic oil blend does not separate and/or crystallize at room temperature in a sealed container for at least 1 week, 2 weeks, 3 weeks, 4 weeks, 5 weeks, 6 weeks, 7 weeks, or 8 weeks. In some embodiments, the therapeutic oil blend does not separate and/or crystallize at room temperature in a sealed container for at least 1 month, 2 months, 3 months, 4 months, 5 months, 6 months, 7 months, or 8 months, 9 months, 10 months, 11 months, or 12 months. In some embodiments, the therapeutic oil blend does not separate and/or crystallize at room temperature in a sealed container for at least 1 year, 2 years, 3 years, 4 years, or 5 years. In some embodiments, the container is not sealed or is not airtight and the therapeutic oil blend still does not separate and/or crystallize for the time periods recited in the foregoing embodiments.

In some embodiments, the improved shelf life of the present therapeutic oil blend is characterized by its improved shelf half-life, as measured through crystallization, separation, or degradation of therapeutic oil blend. The compositions described herein can have a shelf half-life of at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 110, 120, 130, 140, 150, 160, 170, 180, 190, 200, 210, 240, 270, 300, 330, or 360 days. In some cases, the compositions described herein can have a shelf half-life of at least about 1, 2, 3, 4, or 5 years. The nano-penetrative therapeutic oil blend disclosed herein can be characterized by a cannabinoid degradation rate at an ambient temperature of at least 20° C. of at least 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% less than the degradation rate of a non nano-penetrative cannabinoid composition.

In some embodiments, the therapeutic oil blend of the present disclosure has no particles discernible with a microscope at 20× magnification. In some embodiments these properties are maintained at room temperature, and after storage for at least a week. In some embodiments, the therapeutic oil blend has a small particle size, which may facilitate dermal absorption. In some embodiments, the therapeutic oil blend has a mean particle size of less than 10 nm, 20 nm, 30 nm, 40 nm, 50 nm, 60 nm, 70 nm, 80 nm, 90 nm, 100 nm, 110 nm, 120 nm, 130 nm, 140 nm, 150 nm, 160 nm, 170 nm, 180 nm, 190 nm, 200 nm, 210 nm, 220 nm, 230 nm, 240 nm, 250 nm, 260 nm, 270 nm, 280 nm, 290 nm, or 300 nm in diameter (longest diameter in any particle). In some embodiments, the cannabinoid component of the therapeutic oil blend is suspended or contained within phases having a mean particle size of 20-60 nm. In some embodiments, the terpenes of the therapeutic oil blend are suspended or contained within phases having a mean particle size of 20-60 nm. In some embodiments, the particle size refers to the size of the cannabinoid crystal. In some embodiments, the particle size refers to micelle or reverse micelle size. In some embodiments, the therapeutic oil blend is nano-penetrative. In some embodiments, the size of the particles within the therapeutic oil blend, either cannabinoid crystal particles, liposomes, micelles, or reverse micelles, are determined through standard light scatter analysis methodology to have a mean particle size disclosed herein.

In some embodiments, the small particle size leads to increased bioavailability and/or increased bioactivity. The therapeutic oil blends described herein, when administered to a subject, can have improved bioavailability, bioactivity, or both. Bioavailability is the fraction of an administered dosage of unchanged compound that reaches systemic circulation. In some embodiments, the therapeutic oil blend disclosed herein can be characterized by a bioavailability in a subject of at least about 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.5, 4.0, 4.5, 5.0, 5.5, 6.0, 6.5, 7.0, 7.5, 8.0, 8.5, 9.0, 9.5, 10.0 or more times that of a non-nano-penetrative cannabinoid composition, such as traditional CO₂ hemp oil extracts or a mixture of cannabinoids and terpenes without the mechanical agitation employed in preparing the therapeutic oil blends disclosed herein. In some embodiments, the therapeutic oil blend disclosed herein can be characterized by a bioavailability in a subject of at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100, such as traditional CO₂ hemp oil extracts or a mixture of cannabinoids and terpenes without the mechanical agitation employed in preparing the therapeutic oil blends disclosed herein. Bioactivity, or biological activity, is the activity exerted by the active ingredient or ingredients in a composition. In some embodiments, the therapeutic oil blend disclosed herein can be characterized by a bioactivity in a subject of at least about 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.5, 4.0, 4.5, 5.0, 5.5, 6.0, 6.5, 7.0, 7.5, 8.0, 8.5, 9.0, 9.5, 10.0 or more times that of non-nano-penetrative cannabinoid composition, such as traditional CO₂ hemp oil extracts or a mixture of cannabinoids and terpenes without the mechanical agitation employed in preparing the therapeutic oil blends disclosed herein.

In some embodiments, the therapeutic oil blends of the present disclosure exhibit increased skin absorption properties. In some embodiments, the therapeutic oil blends of the present disclosure absorb at least 5%, 10%, 15%, 20%, 25%, 30%, 35%, 40%, 45%, 50%, 55%, 60%, 65%, 70%, 75%, 80%, 85%, 90%, 95%, or 100% faster than a non-nano-penetrative cannabinoid composition, such as traditional CO₂ hemp oil extracts or a mixture of cannabinoids and terpenes without the mechanical agitation employed in preparing the therapeutic oil blends disclosed herein. In some embodiments, the therapeutic oil blends of the present disclosure exhibit at least 5%, 10%, 15%, 20%, 25%, 30%, 35%, 40%, 45%, 50%, 55%, 60%, 65%, 70%, 75%, 80%, 85%, 90%, 95%, or 100% higher visual analog scale scores (e.g., as measured in the example section) than a non-nano-penetrative cannabinoid composition, such as traditional CO₂ hemp oil extracts or a mixture of cannabinoids and terpenes without the mechanical agitation employed in preparing the therapeutic oil blends disclosed herein.

In some embodiments, the therapeutic oil blends of the present disclosure exhibit increased analgesic/anesthetic effects when topically administered. In some embodiments, the therapeutic oil blends of the present disclosure exhibit at least 5%, 10%, 15%, 20%, 25%, 30%, 35%, 40%, 45%, 50%, 55%, 60%, 65%, 70%, 75%, 80%, 85%, 90%, 95%, or 100% greater anesthetic effects than a non-nano-penetrative cannabinoid composition, such as traditional CO₂ hemp oil extracts or a mixture of cannabinoids and terpenes without the mechanical agitation employed in preparing the therapeutic oil blends disclosed herein.

### Formulation of Therapeutic Oil Blend

Dissolving the Cannabinoid

In some embodiments, the present disclosure teaches formulation using a dissolved cannabinoid. In some embodiments, the cannabinoid oil is substantially pure, e.g., substantially free of plant material and other ingredients, such as chlorophyll. In some embodiments, the cannabinoid oil may consist almost exclusively of the cannabinoids of interest without other ingredients. In some embodiments, the cannabinoid oil is a full spectrum hemp oil. In some embodiments the dissolved cannabinoid is in a cannabis extract, such as a hemp CO₂ oil. As noted in other sections of this disclosure, cannabinoids within cannabis extracts can begin to crystallize out of solution shortly after extraction. Therefore, In some embodiments, the formulation of the therapeutic oil blend begins with providing a dissolved cannabinoid-containing oil, referred to as a cannabinoid oil. In some embodiments, the present disclosure refers to cannabinoid oils with fully dissolved cannabinoids as liquid or liquefied cannabinoid oils.

In some embodiments, the cannabinoid oil is already fully dissolved or fully liquified. In some embodiments, the first step is to heat the cannabinoid oil to ensure that it is fully dissolved or fully liquified. In some embodiments, the cannabinoid oil is heated to greater than about 40° C., 45° C., 50° C., 55° C., 60° C., 65° C., 70° C., 75° C., or 80° C. In some embodiments, the oil is heated to a temperature above 40° C. In some embodiments, the cannabinoid oil is heated to about 60° C. Persons having skill in the art will understand that extended heating periods of a cannabinoid composition beyond 80° C. can result in a darker, less commercially desirable product. Therefore in some embodiments, the cannabinoid is not heated beyond 80° C., 90° C., or 100° C., for more than ten minutes, thirty minutes, or one hour. In some embodiments, the cannabinoid is not heated above 80° C.

Emulsion-Like Properties

In some embodiments, the therapeutic oil blends of the present disclosure exhibit emulsion-like properties. For example, in some embodiments, the therapeutic oil blends of the present disclosure exhibit increased stability (e.g., protection from crystallization), and improved bioavailability (e.g., improved topical absorption) that are normally associated with complex cannabinoid emulsions. Additional emulsion-like properties include homogeneity, non-separation, non-precipitation, and uniform distribution of ingredients, e.g., cannabinoid and terpene constituents, throughout the therapeutic oil blend. The therapeutic oil blends of the present disclosure however, do not require the addition of water or water-soluble ingredients, and can thus form in a single oil phase comprising the cannabinoid oil and one or more terpenes or essential oils.

Experiments conducted by the inventor demonstrate that simply blending the ingredients of the therapeutic blend does not produce the desired properties (see examples). Rather, in some embodiments, the therapeutic oil blends require mechanical agitation (e.g., sonication or high pressure homogenization) to impart the desired properties. Without wishing to be bound by any one theory, the inventor hypothesizes that the mechanical agitation of the ingredients causes the cannabinoids to become encapsulated in an emulsion-like manner, in which the cannabinoids are prevented from forming large cannabinoid crystals. Thus, in some embodiments, the therapeutic oil blend may comprise encapsulated cannabinoid. In some embodiments, the therapeutic oil blend may comprise encapsulated cannabinoid generated through the use of liposomes, reverse micelles, or micelles. In some embodiments, the therapeutic oil blend may comprise encapsulated cannabinoid generated via the thorough homogenization of different oil constituents comprised by the therapeutic oil blend.

In some embodiments, the therapeutic oil blend forms a microemulsion or a nanoemulsion, or has microemulsion or nanoemulsion-like characteristics. For example, the therapeutic oil blend in some embodiments comprises droplet, micelle, reverse micelle, liposome, or crystal sizes on the order of less than about 300 nm, 200 nm, or 100 nm. In some embodiments, the emulsion-like therapeutic oil blend is kinetically stable. In some embodiments, the emulsion-like therapeutic oil blend is thermodynamically stable.

In contrast with traditional emulsifications however, the therapeutic oil blend does not, in some embodiments, comprise an exogenous emulsifier. Thus, in some embodiments, the therapeutic oil blend does not comprise an emulsifier or surfactant other than a cannabinoid or a terpene. In some embodiments, the therapeutic oil blend does not comprise a water phase. In some embodiments, the therapeutic oil blend comprises less than 20%, 15%, 10%, 5%, 1%, or 0.1% water soluble ingredients.

In some embodiments, although the therapeutic oil blend is not an emulsion, it can be used to produce other emulsions, such as oil-in-water and water-in-oil emulsions, in which the therapeutic oil blend forms a part of the oil phase. In some embodiments, while the therapeutic oil blend does not comprise an emulsifier other than a cannabinoid or terpene, compositions comprising the therapeutic oil blend may comprise an emulsifier. In some embodiments, compositions comprising the therapeutic oil blend comprise a water phase. Thus, an emulsifier can be used, but is not necessary to produce the therapeutic oil blend of the present disclosure.

Mechanical Agitation

In some embodiments, the present disclosure teaches that the therapeutic oil blends undergo a mechanical agitation step. Persons with ordinary skill in the art will be familiar with the various methods for mechanically agitating formulations. In some embodiments, mechanical agitation is carried out by a sonicator, a tip sonicator, an ultrasonic bath, an ultrasonic homogenizer, or a vortexer. In some embodiments, mechanical agitation can be done by a paint mixer, or other high powered, sustained shaking device. In some embodiments mechanical agitation can be done by a high-speed blender or laboratory macerator. Mechanical agitation includes methods of intense and sustained agitation, distinct from stirring or light shaking. Mechanical agitation does not include hand shaking, stirring, roller bottle mixing, mixing with a magnetic stir bar, or standard rotary shaking. In some embodiments the mechanical agitation can be carried out using a sonicator.

In some embodiments, the present disclosure teaches mechanical agitation that is sufficiently intense and long lasting to cavitate the mixture of ingredients. In some embodiments, cavitation is evidenced by the appearance of bubbles within the mixture. In some embodiments mechanical agitation is sufficient when the resulting product exhibits the increased absorption and/or stability properties of the therapeutic oil blends of the present disclosure.

In some embodiments, any method used for the creation of traditional emulsions can be used to produce the therapeutic oil blends of the present disclosure. In some embodiments, mechanical agitation can be performed with a microfluidic droplet generation device. An exemplary microencapsulation device is described, for example, in U.S. Pat. No. 7,482,152, incorporated herein by reference in its entirety. Droplet size can be controlled by parameters including device geometry, relative flow rates of the fluid streams, and operating pressure.

In some embodiments, the therapeutic oil blend is formulated through the use of sonication. In some embodiments, the therapeutic oil blend is sonicated with an ultrasonic homogenizer. The sonication times, temperatures, amplitude, and frequency may be tuned to obtain smaller particle sizes. The total amount of energy (E) delivered to a suspension not only depends on the applied power (P) but also on the total amount of time (t) that the suspension is subject to the ultrasonic treatment: E=P×t. In some embodiments the cannabinoid-containing oil (also referred to herein as the cannabinoid oil), e.g. hemp oil, is heated prior to initial sonication. The formation of cavitation bubbles will increase the temperature of the sonicated medium, such that temperature limits should be set throughout sonication to ensure a reasonable temperature range during formulation. during this process. calorimetry can be used to measure the effective acoustic energy delivered to a sonicated liquid in a device independent manner. The method is based on the measurement of the temperature increase in a liquid medium over time as a result of cavitation induced in a liquid by an immersed ultrasound probe. At a given device output power setting, the temperature increase in the liquid is recorded over time and the effective delivered power can be calculated using the following equation: P=((dT/dt)MCp) where P is the delivered acoustic power (W), T and t are temperature (K) and time (s), respectively, Cp is the specific heat of the liquid (J/g·K) and M is the mass of liquid (g). In some embodiments, the power, frequency, and on and off intervals are tuned to achieve a desired ultrasonic amplitude, cavitation intensity, time spent in the cavitation zone, etc. The power ranges, frequency ranges, and on and off intervals disclosed in some embodiments herein are provided based on a 100 g therapeutic oil blend sample size illustrative embodiment, and are not intended to be limiting.

In some embodiments, during the sonication process for the preparation of the therapeutic oil blend, cooling steps and/or measures are taken to maintain the temperature of the mixture at a desired level. For cooling the mixture throughout the sonication process, cooling baths, ice water baths, fans, cooling sleeves, cooling probes, cooling chambers, refrigeration, freezing, heat dispersion modules, and the like may be employed. In some embodiments, an ingredient or mixture is cooled prior to sonication. In some embodiments, an ingredient or mixture is cooled prior to addition to a mixture. In some embodiments, an ingredient or mixture of ingredients is cooled during sonication. In some embodiments, the sonicated cannabinoid-comprising oil is down to at least room temperature before homogenizing the other terpenes into the cannabinoid-comprising oil.

In some embodiments, foaming is avoided through the use of lower power settings following the addition of any surfactant or other ingredient with foaming properties, e.g., some terpenes.

In some embodiments, the ingredients of the therapeutic oil are mixed and mechanically agitated. In some embodiments, two or more ingredients of the therapeutic oil are mixed and mechanically agitated together. In some embodiments, the cannabinoid oil is mechanically agitated alone before adding any other ingredients. In some embodiments, ingredients are each added with their own mechanical agitation cycles. In some embodiments the clove oil and peppermint oil are each added with their own mechanical agitation cycles. In some embodiments, eugenol is added individually with its own mechanical agitation cycle. In some embodiments, d-limonene is added individually with its own mechanical agitation cycle. Exemplary ranges of sonication powers, time intervals, and frequencies are provided herein based on those used with an ultrasonic homogenizer with a power range of 80-1800 W and a frequency range of 19-22 kHz, with sample volumes in the range of 10-200 mL. These illustrative settings may be adapted based on known principles to the use of other mechanical agitators or sample sizes.

In some embodiments, the first step is to mechanically agitate (e.g., sonicate) the fully dissolved cannabinoid oil. In some embodiments, the cannabinoid oil is mechanically agitated for about 0.2, 0.3, 0.4, 0.5, 0.6, 0.7, 0.8, 0.9, 1, 2, 3, 4, or 5 hours of active agitation (e.g., sonication) time, including any ranges or subranges therebetween. In some embodiments, the cannabinoid oil is mechanically agitated (e.g., sonicated) for about 2 hours of active sonication time. In some embodiments, the cannabinoid oil is mechanically agitated until the desired level of cavitation has been achieved.

In some embodiments, the mechanical agitation is sonication, and the sonication is carried out between 15-25 kHz and at a power level of between 1500 and 2000 W. In some embodiments, the initial sonication of the cannabinoid oil is carried out at about 22 kHz and/or at about 1800 W for ˜50 g, which settings may be scaled according to known practices for larger sample sizes. In some embodiments, the sonication step can raise the temperature and cause the cannabinoid oil to overheat. In some embodiments, the present disclosure teaches keeping the cannabinoid oil below 80° C. In some embodiments, the mechanical agitation of the cannabinoid oil is sonication, and the sonication alternates between on and off periods, e.g., 5-60 seconds on and 5-60 seconds off, which range is merely intended to illustrate, and not to limit, the possible sonication intervals that may be employed within the present methods. A person of skill in the art recognizes that different on/off intervals are effective to achieve a desired level or acceptable range of intensity of sonication, power delivered to the ingredients being sonicated, or temperature increase induced by sonication. These exemplary parameters are disclosed based on observations for a sample size of approximately ˜60 g and may be adjusted according to known principles for larger or smaller scale samples.

In some embodiments, following the sonication of the cannabinoid oil, the cannabinoid oil is cooled. In some embodiments, the cannabinoid oil is brought down to room temperature, or around 15° C.-30° C. In some embodiments, the sonicated cannabinoid oil is brought to about 23° C. In some embodiments, the cannabinoid oil is brought to less than about 40° C.

In some embodiments, terpenes and/or essential oils are added to the cannabinoid oil, e.g., the mechanically agitated cannabinoid oil or the mechanically agitated and cooled cannabinoid oil. In some embodiments, the present disclosure teaches homogenizing the terpenes into the cannabinoid oil via mechanical agitation, e.g., sonication.

In some embodiments, the present disclosure teaches that any desired penetrative aids, vasodilation agents, anti-inflammatory agents, analgesic compounds, and anesthetic compounds may be added simultaneously or serially to the cannabinoid oil. In some embodiments, one or more ingredients are added to the cannabinoid oil during rest. In some embodiments, one or more ingredients are added to the cannabinoid oil during mechanical agitation. In some embodiments, particular temperatures, power settings, and order of operations may be employed to maximize desirable properties, such as particle size and homogeneity. The particular settings disclosed herein are intended to illustrate, and not to limit, the disclosed methods of formulating the therapeutic oil blends of the disclosure, which may be produced with a range of effective settings, temperatures and steps.

In some embodiments, eugenol (or clove bud oil, which comprises greater than 80% eugenol) is added to the cannabinoid oil. In some embodiments, eugenol is added to the mechanically agitated, e.g., sonicated, cannabinoid oil. In some embodiments, eugenol is the first terpene/essential oil to be added or one of the first terpenes/essential oils to be added to the cannabinoid oil. In some embodiments, eugenol is chilled prior to addition. In some embodiments, eugenol is chilled to less than about 0° C. before addition. In some embodiments, the temperature of the mixture is maintained below about 90, 80, 70, 60, 50, 40, 30, or 20° C. during mechanical agitation. In some embodiments, the temperature is maintained below about 60° C. during mechanical agitation. In some embodiments, eugenol is added during active mechanical agitation, e.g. sonication. In some embodiments, sonication is maintained at a level of about 15-25 kHz, alternating between on and off periods. In some embodiments, sonication is carried out at around 20 kHz. In some embodiments, the power level of sonication is set to about 500-1000 W. In some embodiments, the homogenization of the cannabinoid oil and eugenol is carried out for 5-30 minutes, e.g., 10 minutes of active sonication time. In some embodiments, the sonication alternates on a 5-60 seconds on, 1-30 seconds off cycle. These exemplary parameters are disclosed based on observations for a sample size of approximately 90 g and may be adjusted according to known principles for larger or smaller scale samples.

In some embodiments, d-limonene is added individually to the therapeutic oil blend. In some embodiments, d-limonene is added to the therapeutic oil blend following the addition of eugenol. D-limonene may be used to increase the dermal absorption characteristics of the therapeutic oil blend. In some embodiments, d-limonene is added slowly during active mechanical agitation, e.g., sonication. In some embodiments, the temperature is maintained below about 80, 70, 60, 50, 40, 30, or 20° C. during mechanical agitation. In some embodiments, the temperature is maintained below about 40° C. during mechanical agitation. In some embodiments, sonication is maintained at a level of about 15-25 kHz, alternating between on and off periods. In some embodiments, sonication is carried out at around 22 kHz. In some embodiments, the power level of sonication is set to about 1500-2000 W. In some embodiments, the homogenization of the cannabinoid oil mixture and d-limonene is carried out for 5-30 minutes, e.g., 15 minutes of active sonication time. In some embodiments, the sonication alternates 30-120 seconds on, 5-60 seconds off. These exemplary parameters are disclosed based on observations for a sample size of approximately 90 g and may be adjusted according to known principles for larger or smaller scale samples.

In some embodiments, additional terpenes, such as menthol and menthone, are added serially to the cannabinoid oil. In some embodiments, d-limonene, menthol, and menthone are added at the same time. In some embodiments, the terpenes are added via an essential oil comprising the terpenes. For example, In some embodiments, d-limonene, menthol, and menthone are added simultaneously to the sample in the form of peppermint oil. In some embodiments, one or more terpenes or essential oils are added to the cannabinoid oil after mechanical agitation of the cannabinoid oil. In some embodiments, one or more terpenes or essential oils are added to the cannabinoid oil after addition of eugenol. In some embodiments, one or more terpenes or essential oils are added to the cannabinoid oil after addition of eugenol and sonication of the cannabinoid oil and eugenol. In some embodiments, one or more terpenes or essential oils are added to the cannabinoid oil after addition of both eugenol and d-limonene.

In some embodiments, one or more terpenes or essential oils are added slowly during active mechanical agitation, e.g., sonication. In some embodiments, these ingredients are chilled prior to addition. In some embodiments, sonication is maintained at a level of about 15-25 kHz, alternating between on and off periods. In some embodiments, sonication is carried out at around 19 kHz. In some embodiments, the power level of sonication is set to about 100-500 W. In some embodiments, the homogenization of the cannabinoid oil mixture and the additional ingredients is carried out for 5-30 minutes, e.g., 15 minutes of active sonication time. In some embodiments, the sonication alternates on a 1-30 s/5-60 s interval of on/off. In some embodiments, the temperature is maintained below about 60, 55, 50, 45, or 40° C. during mechanical agitation. These exemplary parameters are disclosed based on observations for a sample size of approximately 100 g and may be adjusted according to known principles for larger or smaller scale samples.

In some embodiments, after the addition of each of the constituents, the cannabinoid oil plus terpene and/or essential oil mixture is further mechanically agitated, e.g., sonicated, for an additional period of time to produce the fully homogenized therapeutic oil blend with the desired average particle size. In some embodiments, the temperature of the sample is maintained below about 80, 70, 60, 50, or 40° C. during sonication. In some embodiments, the temperature of the sample is maintained below about 60° C. during mechanical agitation. In some embodiments, sonication is maintained at a level of about 15-25 kHz, alternating between on and off periods. In some embodiments, sonication is carried out at around 22 kHz. In some embodiments, the power level of sonication is set to about 1500-2000 W. In some embodiments, the homogenization of the cannabinoid oil mixture is carried out for 20-60 minutes, e.g., 30 minutes of active sonication time. In some embodiments, the sonication alternates on a 15-90 s/30-240 s interval of on/off. These exemplary parameters are disclosed based on observations for a sample size of approximately 100 g and may be adjusted according to known principles for larger or smaller scale samples.

Throughout the above steps, in some embodiments, the present disclosure teaches regulating the temperature of the oils and mixtures throughout the homogenization process leading to the formation of the therapeutic oil blend. In some embodiments, the present disclosure teaches keeping the temperature of the oils and mixtures below a pre-determined set point. In some embodiments, the set point may be fixed throughout the homogenization process. In some embodiments, the set point may vary in different steps.

In some embodiments, the set point is between 30° C.-90° C., i.e., the mixture is maintained below a temperature within this range. In some embodiments, the set point is 60° C. for some steps. In some embodiments, the set point is 40° C. for some steps. Temperature may be regulated by a variety of means, e.g., ice bath, water bath, coolant, fans, refrigeration, chilling, resting to allow ambient temperature to return, etc.

In addition, measures may be taken to avoid foaming after addition of surfactants (e.g., terpenes with surfactant properties). In some embodiments, long rest periods are employed to avoid foaming in the presence of surfactants. Mechanical agitation may also be employed in pulse mode to help avoid foaming. In some embodiments, sonication is generally carried out in alternating periods of on and off, e.g., 10/10, 30/5, 60/20, 5/15, 45/120 in terms of on seconds/off seconds. The present methods are not limited to any particular alternation of sonication or particular period of time. The times may be determined by one of skill in the art in order to maintain temperature below the set point, avoid foaming, increase homogenization, and the like. Longer rest periods may be employed to maintain a lower temperature and/or decrease foaming. The sonication power and frequency may be adjusted as well to improve one or more of these parameters.

The foregoing steps may be used to create a therapeutic oil blend of the present disclosure with small particle size (e.g. below 60 nm average), nano-penetrative qualities, and/or non-crystallizing qualities. Comparatively, the same base hemp oil and the same base ingredients mixed without mechanical agitation would result in a product that would crystallize within 24 hours, would not absorb appropriately, would feel sticky on the skin, and would not absorb into the skin quickly.

### Exemplary Therapeutic Oil Blends

The present disclosure provides methods of formulating therapeutic oil blends comprising at least one cannabinoid and at least one terpene.

In some embodiments the therapeutic oil blends of the present disclosure comprise a total w/w terpene content of greater or less than 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, or 90%, including all ranges and subranges therebetween. Thus in some embodiments, the therapeutic oil blends of the present disclosure comprise a total w/w terpene content between 20%-90% w/w, including all ranges and subranges therebetween.

In some embodiments the therapeutic oil blends of the present disclosure comprise greater than or less than 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90% w/w of each terpene in the composition, including all ranges and subranges. Thus in some embodiments, each terpene within the therapeutic oil blend is at a w/w concentration between 0.1%-90% w/w, including all ranges and subranges therebetween. This paragraph should be interpreted as providing the possible concentrations of any of the terpenes in the therapeutic oil blend, including those listed in the terpene section of this document, and those listed immediately below.

In some embodiments, the therapeutic oil blend comprises a terpene selected from the group consisting of bisabolol, borneol, caryophyllene, carene, camphene, camphor, cineol, citronellal, eucalyptol, eugenol, geraniol, guaiol, humulene, isopropyltoluene, isopulegol, linalool, d-limonene, menthol, menthone, beta-myrcene, nerolidol, ocimene, alpha-pinene, beta-pinene, phytol, pulegone, alpha-terpinene, gamma-terpinene, terpinolene, and thymol. In some embodiments, the therapeutic oil blend comprises eugenol. In some embodiments, the therapeutic oil blend comprises menthol. In some embodiments, the therapeutic oil blend comprises menthone. In some embodiments, the therapeutic oil blend comprises d-limonene. In some embodiments, the therapeutic oil blend comprises eugenol, menthol, menthone, and d-limonene. In some embodiments, the therapeutic oil blend comprises eugenol, menthol, menthone, and d-limonene and at least one more, two more, three more, or four more terpenes selected from the list consisting of bisabolol, borneol, caryophyllene, carene, camphene, camphor, cineol, citronellal, eucalyptol, geraniol, guaiol, humulene, isopropyltoluene, isopulegol, linalool, beta-myrcene, nerolidol, ocimene, alpha-pinene, beta-pinene, phytol, pulegone, alpha-terpinene, gamma-terpinene, terpinolene, and thymol.

In some embodiments, the terpenes of the present disclosure are provided in the form of essential oils. Thus, in some embodiments the therapeutic oil blends of the present disclosure comprise a total w/w essential oil content of greater or less than 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, or 90%, including all ranges and subranges therebetween. Thus in some embodiments, the therapeutic oil blends of the present disclosure comprise a total w/w essential oil content between 20%-90% w/w, including all ranges and subranges therebetween.

In some embodiments the therapeutic oil blends of the present disclosure comprise greater than or less than 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90% w/w of each essential in the composition, including all ranges and subranges. Thus in some embodiments, each essential oil within the therapeutic oil blend is at a w/w concentration between 0.1%-90% w/w, including all ranges and subranges therebetween. This paragraph should be interpreted as providing the possible concentrations of any of the essential oils in the therapeutic oil blend, including those listed in the essential oil section of this document, and those listed immediately below.

In some embodiments, the therapeutic oil blend comprises one or more essential oils. In some embodiments, the therapeutic oil blend comprises one or more essential oils selected from the list consisting of clove oil, clove bud oil, clove leaf oil, basil oil, lemon balm oil, cinnamon oil, nutmeg oil, Japanese star anise oil, spearmint oil, peppermint oil, wintergreen oil, pennyroyal oil, vanilla bean oil, cornmint oil, and combinations thereof. In some embodiments, the therapeutic oil blend comprises about 0.1, 0.2, 0.3, 0.4, 0.5, 0.6, 0.7, 0.8, 0.9, 1, 2, 3, 4, 5, 6, 7, 8, 9, 10, 11, 12, 13, 14 15, 16, 17, 18, 19, 20, 21, 22, 23, 24, 25, 26, 27, 28, 29, 30, 40, 45, 50, 55, 60, 65, 70, 75, 80, 85, 90, 95% w/w, or any ranges or subranges therebetween, of an essential oil or blend of essential oils.

In some embodiments, the therapeutic oil blend comprises clove oil, e.g., clove bud oil. In some embodiments, the therapeutic oil blend comprises about 1, 5, 10, 15, 20, 25, 30, 40, 45, 50, 55, 60, 65, 70, 75, 80, 85, 90, 95% w/w, or any ranges or subranges therebetween, of clove oil. In some embodiments, the therapeutic oil blend comprises about 20-40% w/w clove oil. In some embodiments, the therapeutic oil blend comprises about 30% w/w clove oil.

In some embodiments, the therapeutic oil blend comprises peppermint oil. In some embodiments, the therapeutic oil blend comprises about 1, 5, 10, 15, 20, 25, 30, 40, 45, 50, 55, 60, 65, 70, 75, 80, 85, 90, 95% w/w, or any ranges or subranges therebetween, of peppermint oil. In some embodiments, the therapeutic oil blend comprises about 5-20% w/w peppermint oil. In some embodiments, the therapeutic oil blend comprises about 10% w/w peppermint oil.

In some embodiments the therapeutic oil blends of the present disclosure comprise a total w/w cannabinoid content of greater or less than 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, or 90%, including all ranges and subranges therebetween. Thus in some embodiments, the therapeutic oil blends of the present disclosure comprise a total w/w cannabinoid content between 20%-90%, including all ranges and subranges therebetween.

In some embodiments the therapeutic oil blends of the present disclosure comprise greater than or less than 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90% w/w of each cannabinoid in the composition, including all ranges and subranges. Thus in some embodiments, each cannabinoid within the therapeutic oil blend is at a w/w concentration between 0.1%-90%, including all ranges and subranges therebetween. This paragraph should be interpreted as providing the possible concentrations of any of the cannabinoids in the therapeutic oil blend, including those listed in the cannabinoid section of this document, and those listed immediately below.

In some embodiments, the cannabinoid is a cannabidiol-related cannabinoid. In some embodiments, the cannabidiol-related cannabinoid is selected from the group consisting of cannabidiolic acid (CBDA), cannabidiol (CBD), cannabidiol monomethylether (CBDM), cannabidiol-C4 (CBD-C4), cannabidivarinic acid (CBDVA), cannabidivarin (CBDV), cannabidiorcol (CBD-C1), a salt thereof, a derivative thereof, and a mixture thereof. In some embodiments, the cannabidiol-related cannabinoid is CBD.

In some embodiments, the cannabinoid is cannabidiol (CBD).

In some embodiments, the therapeutic oil blend comprises 25%-75% w/w cannabinoids. In some embodiments, the therapeutic oil blend comprises at least 25%, at least 30%, at least 35%, at least 40%, at least 45%, at least 50%, at least 55%, at least 60%, at least 65%, at least 70%, or at least 75% w/w cannabinoids. In some embodiments, the therapeutic oil blend comprises about 25%, about 30%, about 35%, about 40%, about 45%, about 50%, about 55%, about 60%, about 65%, about 70%, or about 75% w/w cannabinoids. In some embodiments, the therapeutic oil blend comprises at most 25%, at most 30%, at most 35%, at most 40%, at most 45%, at most 50%, at most 55%, at most 60%, at most 65%, at most 70%, or at most 75% w/cannabinoids. In some embodiments, the therapeutic oil blend comprises 25%-35% w/w cannabinoids. In some embodiments, the therapeutic oil blend comprises 35%-45% w/w cannabinoids. In some embodiments, the therapeutic oil blend comprises 45%-55% w/w cannabinoids. In some embodiments, the therapeutic oil blend comprises 55%-65% w/w cannabinoids. In some embodiments, the therapeutic oil blend comprises 65%-75% w/w cannabidiol-related cannabinoid.

In some embodiments, the therapeutic oil blend comprises CBD. In some embodiments, the therapeutic oil blend comprises 25%-75% w/w CBD. In some embodiments, the therapeutic oil blend comprises at least 25%, at least 30%, at least 35%, at least 40%, at least 45%, at least 50%, at least 55%, at least 60%, at least 65%, at least 70%, or at least 75% w/w CBD. In some embodiments, the therapeutic oil blend comprises about 25%, about 30%, about 35%, about 40%, about 45%, about 50%, about 55%, about 60%, about 65%, about 70%, or about 75% w/w CBD. In some embodiments, the therapeutic oil blend comprises at most 25%, at most 30%, at most 35%, at most 40%, at most 45%, at most 50%, at most 55%, at most 60%, at most 65%, at most 70%, or at most 75% w/w CBD. In some embodiments, the therapeutic oil blend comprises 25%-35% w/w CBD. In some embodiments, the therapeutic oil blend comprises 35%-45% w/w CBD. In some embodiments, the therapeutic oil blend comprises 45%-55% w/w CBD. In some embodiments, the therapeutic oil blend comprises 55%-65% w/w CBD. In some embodiments, the therapeutic oil blend comprises 65%-75% w/w CBD.

In some embodiments, the therapeutic oil blend comprises hemp oil, a CBD extract, a CBD isolate, or any combination thereof. In some embodiments, the therapeutic oil blend comprises about 1, 5, 10, 15, 20, 25, 30, 40, 45, 50, 55, 60, 65, 70, 75, 80, 85, 90, 95% w/w, or any ranges or subranges therebetween, of a hemp oil, CBD extract, or CBD isolate. In some embodiments, the therapeutic oil blend comprises about 30-90% w/w of a hemp oil, CBD extract, or CBD isolate. In some embodiments, the therapeutic oil blend comprises about 60% w/w of a hemp oil, CBD extract, or CBD isolate. In some embodiments, the hemp oil, CBD extract, or CBD isolate has a concentration of CBD greater than 50, 60, 65, 70, 75, 80, 85, or 90% w/w. In some embodiments, the hemp oil, CBD extract, or CBD isolate has a concentration of CBD greater than 80% w/w.

In some embodiments the therapeutic oil blends of the present disclosure comprise one or more non-cannabinoid or terpene ingredients. For example, in some embodiments, the therapeutic oil blends of the present disclosure comprise a carrier oil. In some embodiments, the therapeutic oil blend comprises about 30% to about 99% by weight of a mixture of the cannabinoids and terpenes. In some embodiments, the therapeutic oil blend comprises about 40% to about 95% by weight of a mixture of the cannabinoids and terpenes. In some embodiments, the therapeutic oil blend comprises about 50% to about 95% by weight of a mixture of the cannabinoids and terpenes. In some embodiments, the therapeutic oil blend comprises about 60% to about 95% by weight of a mixture of the cannabinoids and terpenes. In some embodiments, the therapeutic oil blend comprises about 70% to about 95% by weight of a mixture of the cannabinoids and terpenes. In some embodiments, the therapeutic oil blend comprises about 80% to about 95% by weight of a mixture of the cannabinoids and terpenes.

In some embodiments, the therapeutic oil blend comprises camphor. In some embodiments, the therapeutic oil blend comprises 0.05-10% w/w camphor. In some embodiments, the therapeutic oil blend comprises 0.05-0.5% w/w camphor. In some embodiments, the therapeutic oil blend comprises 0.1-1% w/w camphor. In some embodiments, the therapeutic oil blend comprises 1-5% w/w camphor. In some embodiments, the therapeutic oil blend comprises 5-10% w/w camphor. In some embodiments, the therapeutic oil blend comprises at least 0.05%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1.0%, 1.5%, 2.0%, 2.5%, 3.0%, 3.5%, 4.0%, 4.5%, 5.5%, 6.0%, 6.5%, 7.0%, 7.5%, 8.0%, 8.5%, 9.0%, 9.5%, or 10.0% w/w camphor. In some embodiments, the therapeutic oil blend comprises about 0.05%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1.0%, 1.5%, 2.0%, 2.5%, 3.0%, 3.5%, 4.0%, 4.5%, 5.5%, 6.0%, 6.5%, 7.0%, 7.5%, 8.0%, 8.5%, 9.0%, 9.5%, or 10.0% w/w camphor. In some embodiments, the therapeutic oil blend comprises at most 0.05%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1.0%, 1.5%, 2.0%, 2.5%, 3.0%, 3.5%, 4.0%, 4.5%, 5.5%, 6.0%, 6.5%, 7.0%, 7.5%, 8.0%, 8.5%, 9.0%, 9.5%, or 10.0% w/w camphor.

In some embodiments, the therapeutic oil blend comprises eucalyptol. In some embodiments, the therapeutic oil blend comprises 0.01-10% w/w eucalyptol. In some embodiments, the therapeutic oil blend comprises 0.01-0.05% w/w eucalyptol. In some embodiments, the therapeutic oil blend comprises 0.05-0.1% w/w eucalyptol. In some embodiments, the therapeutic oil blend comprises 0.1-0.5% w/w eucalyptol. In some embodiments, the therapeutic oil blend comprises 0.5-1% w/w eucalyptol. In some embodiments, the therapeutic oil blend comprises 1-5% w/w eucalyptol. In some embodiments, the therapeutic oil blend comprises 5-10% w/w eucalyptol. In some embodiments, the therapeutic oil blend comprises at least 0.01%, 0.02%, 0.03%, 0.04%, 0.05%, 0.06%, 0.07%, 0.08%, 0.09%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1.0%, 1.5%, 2.0%, 2.5%, 3.0%, 3.5%, 4.0%, 4.5%, 5.5%, 6.0%, 6.5%, 7.0%, 7.5%, 8.0%, 8.5%, 9.0%, 9.5%, or 10.0% w/w eucalyptol. In some embodiments, the therapeutic oil blend comprises about 0.01%, 0.02%, 0.03%, 0.04%, 0.05%, 0.06%, 0.07%, 0.08%, 0.09%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1.0%, 1.5%, 2.0%, 2.5%, 3.0%, 3.5%, 4.0%, 4.5%, 5.5%, 6.0%, 6.5%, 7.0%, 7.5%, 8.0%, 8.5%, 9.0%, 9.5%, or 10.0% w/w eucalyptol. In some embodiments, the therapeutic oil blend comprises at most 0.01%, 0.02%, 0.03%, 0.04%, 0.05%, 0.06%, 0.07%, 0.08%, 0.09%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1.0%, 1.5%, 2.0%, 2.5%, 3.0%, 3.5%, 4.0%, 4.5%, 5.5%, 6.0%, 6.5%, 7.0%, 7.5%, 8.0%, 8.5%, 9.0%, 9.5%, or 10.0% w/w eucalyptol.

In some embodiments, the therapeutic oil blend comprises eugenol. In some embodiments, the therapeutic oil blend comprises 5-50% w/w eugenol. In some embodiments, the therapeutic oil blend comprises 5-10% w/w eugenol. In some embodiments, the therapeutic oil blend comprises 10-15% w/w eugenol. In some embodiments, the therapeutic oil blend comprises 15-20% w/w eugenol. In some embodiments, the therapeutic oil blend comprises 20-25% w/w eugenol. In some embodiments, the therapeutic oil blend comprises 25-30% w/w eugenol. In some embodiments, the therapeutic oil blend comprises 30-35% w/w eugenol. In some embodiments, the therapeutic oil blend comprises 35-40% w/w eugenol. In some embodiments, the therapeutic oil blend comprises 40-45% w/w eugenol. In some embodiments, the therapeutic oil blend comprises 45-50% w/w eugenol. In some embodiments, the therapeutic oil blend comprises at least 5%, 6%, 7%, 8%, 9%, 10.0%, 15%, 20%, 25%, 30%, 35%, 40%, 45%, or 50% w/w eugenol. In some embodiments, the therapeutic oil blend comprises about 5%, 6%, 7%, 8%, 9%, 10.0%, 15%, 20%, 25%, 30%, 35%, 40%, 45%, or 50% w/w eugenol. In some embodiments, the therapeutic oil blend comprises at most 5%, 6%, 7%, 8%, 9%, 10.0%, 15%, 20%, 25%, 30%, 35%, 40%, 45%, or 50% w/w eugenol. In some embodiments, the eugenol comprised by the therapeutic oil blend is provided in the form of clove bud oil.

In some embodiments, the therapeutic oil blend comprises linalool. In some embodiments, the therapeutic oil blend comprises 0.01-10% w/w linalool. In some embodiments, the therapeutic oil blend comprises 0.01-0.05% w/w linalool. In some embodiments, the therapeutic oil blend comprises 0.05-0.1% w/w linalool. In some embodiments, the therapeutic oil blend comprises 0.1-0.5% w/w linalool. In some embodiments, the therapeutic oil blend comprises 0.5-1% w/w linalool. In some embodiments, the therapeutic oil blend comprises 1-5% w/w linalool. In some embodiments, the therapeutic oil blend comprises 5-10% w/w linalool. In some embodiments, the therapeutic oil blend comprises at least 0.01%, 0.02%, 0.03%, 0.04%, 0.05%, 0.06%, 0.07%, 0.08%, 0.09%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1.0%, 1.5%, 2.0%, 2.5%, 3.0%, 3.5%, 4.0%, 4.5%, 5.5%, 6.0%, 6.5%, 7.0%, 7.5%, 8.0%, 8.5%, 9.0%, 9.5%, or 10.0% w/w linalool. In some embodiments, the therapeutic oil blend comprises about 0.01%, 0.02%, 0.03%, 0.04%, 0.05%, 0.06%, 0.07%, 0.08%, 0.09%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1.0%, 1.5%, 2.0%, 2.5%, 3.0%, 3.5%, 4.0%, 4.5%, 5.5%, 6.0%, 6.5%, 7.0%, 7.5%, 8.0%, 8.5%, 9.0%, 9.5%, or 10.0% w/w linalool. In some embodiments, the therapeutic oil blend comprises at most 0.01%, 0.02%, 0.03%, 0.04%, 0.05%, 0.06%, 0.07%, 0.08%, 0.09%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1.0%, 1.5%, 2.0%, 2.5%, 3.0%, 3.5%, 4.0%, 4.5%, 5.5%, 6.0%, 6.5%, 7.0%, 7.5%, 8.0%, 8.5%, 9.0%, 9.5%, or 10.0% w/w linalool.

In some embodiments, the therapeutic oil blend comprises d-limonene. In some embodiments, the therapeutic oil blend comprises 0.05-10% w/w d-limonene. In some embodiments, the therapeutic oil blend comprises 0.05-0.5% w/w d-limonene. In some embodiments, the therapeutic oil blend comprises 0.1-1% w/w d-limonene. In some embodiments, the therapeutic oil blend comprises 1-5% w/w d-limonene. In some embodiments, the therapeutic oil blend comprises 5-10% w/w d-limonene. In some embodiments, the therapeutic oil blend comprises at least 0.05%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1.0%, 1.5%, 2.0%, 2.5%, 3.0%, 3.5%, 4.0%, 4.5%, 5.5%, 6.0%, 6.5%, 7.0%, 7.5%, 8.0%, 8.5%, 9.0%, 9.5%, or 10.0% w/w d-limonene. In some embodiments, the therapeutic oil blend comprises about 0.05%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1.0%, 1.5%, 2.0%, 2.5%, 3.0%, 3.5%, 4.0%, 4.5%, 5.5%, 6.0%, 6.5%, 7.0%, 7.5%, 8.0%, 8.5%, 9.0%, 9.5%, or 10.0% w/w d-limonene. In some embodiments, the therapeutic oil blend comprises at most 0.05%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1.0%, 1.5%, 2.0%, 2.5%, 3.0%, 3.5%, 4.0%, 4.5%, 5.5%, 6.0%, 6.5%, 7.0%, 7.5%, 8.0%, 8.5%, 9.0%, 9.5%, or 10.0% w/w d-limonene.

In some embodiments, the therapeutic oil blend comprises menthol. In some embodiments, the therapeutic oil blend comprises 0.1-10% w/w menthol. In some embodiments, the therapeutic oil blend comprises 0.1-1% w/w menthol. In some embodiments, the therapeutic oil blend comprises 1-5% w/w menthol. In some embodiments, the therapeutic oil blend comprises 5-10% w/w menthol. In some embodiments, the therapeutic oil blend comprises at least 0.1%, 0.2%, 0.3%, 0.4%, 0.5%, 0.6%, 0.7%, 0.8%, 0.9%, 1.0%, 1.5%, 2.0%, 2.5%, 3.0%, 3.5%, 4.0%, 4.5%, 5.5%, 6.0%, 6.5%, 7.0%, 7.5%, 8.0%, 8.5%, 9.0%, 9.5%, or 10.0% w/w menthol. In some embodiments, the therapeutic oil blend comprises about 0.1%, 0.2%, 0.3%, 0.4%, 0.5%, 0.6%, 0.7%, 0.8%, 0.9%, 1.0%, 1.5%, 2.0%, 2.5%, 3.0%, 3.5%, 4.0%, 4.5%, 5.5%, 6.0%, 6.5%, 7.0%, 7.5%, 8.0%, 8.5%, 9.0%, 9.5%, or 10.0% w/w menthol. In some embodiments, the therapeutic oil blend comprises at most 0.1%, 0.2%, 0.3%, 0.4%, 0.5%, 0.6%, 0.7%, 0.8%, 0.9%, 1.0%, 1.5%, 2.0%, 2.5%, 3.0%, 3.5%, 4.0%, 4.5%, 5.5%, 6.0%, 6.5%, 7.0%, 7.5%, 8.0%, 8.5%, 9.0%, 9.5%, or 10.0% w/w menthol.

In some embodiments, the therapeutic oil blend comprises menthone. In some embodiments, the therapeutic oil blend comprises 0.1-10% w/w menthone. In some embodiments, the therapeutic oil blend comprises 0.1-1% w/w menthone. In some embodiments, the therapeutic oil blend comprises 1-5% w/w menthone. In some embodiments, the therapeutic oil blend comprises 5-10% w/w menthone. In some embodiments, the therapeutic oil blend comprises at least 0.1%, 0.2%, 0.3%, 0.4%, 0.5%, 0.6%, 0.7%, 0.8%, 0.9%, 1.0%, 1.5%, 2.0%, 2.5%, 3.0%, 3.5%, 4.0%, 4.5%, 5.5%, 6.0%, 6.5%, 7.0%, 7.5%, 8.0%, 8.5%, 9.0%, 9.5%, or 10.0% w/w menthone. In some embodiments, the therapeutic oil blend comprises about 0.1%, 0.2%, 0.3%, 0.4%, 0.5%, 0.6%, 0.7%, 0.8%, 0.9%, 1.0%, 1.5%, 2.0%, 2.5%, 3.0%, 3.5%, 4.0%, 4.5%, 5.5%, 6.0%, 6.5%, 7.0%, 7.5%, 8.0%, 8.5%, 9.0%, 9.5%, or 10.0% w/w menthone. In some embodiments, the therapeutic oil blend comprises at most 0.1%, 0.2%, 0.3%, 0.4%, 0.5%, 0.6%, 0.7%, 0.8%, 0.9%, 1.0%, 1.5%, 2.0%, 2.5%, 3.0%, 3.5%, 4.0%, 4.5%, 5.5%, 6.0%, 6.5%, 7.0%, 7.5%, 8.0%, 8.5%, 9.0%, 9.5%, or 10.0% w/w menthone.

In some embodiments, the therapeutic oil blend comprises beta-myrcene. In some embodiments, the therapeutic oil blend comprises 0.05-10% w/w beta-myrcene. In some embodiments, the therapeutic oil blend comprises 0.05-0.5% w/w beta-myrcene. In some embodiments, the therapeutic oil blend comprises 0.1-1% w/w beta-myrcene. In some embodiments, the therapeutic oil blend comprises 1-5% w/w beta-myrcene. In some embodiments, the therapeutic oil blend comprises 5-10% w/w beta-myrcene. In some embodiments, the therapeutic oil blend comprises at least 0.05%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1.0%, 1.5%, 2.0%, 2.5%, 3.0%, 3.5%, 4.0%, 4.5%, 5.5%, 6.0%, 6.5%, 7.0%, 7.5%, 8.0%, 8.5%, 9.0%, 9.5%, or 10.0% w/w beta-myrcene. In some embodiments, the therapeutic oil blend comprises about 0.05%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1.0%, 1.5%, 2.0%, 2.5%, 3.0%, 3.5%, 4.0%, 4.5%, 5.5%, 6.0%, 6.5%, 7.0%, 7.5%, 8.0%, 8.5%, 9.0%, 9.5%, or 10.0% w/w beta-myrcene. In some embodiments, the therapeutic oil blend comprises at most 0.05%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1.0%, 1.5%, 2.0%, 2.5%, 3.0%, 3.5%, 4.0%, 4.5%, 5.5%, 6.0%, 6.5%, 7.0%, 7.5%, 8.0%, 8.5%, 9.0%, 9.5%, or 10.0% w/w beta-myrcene.

In some embodiments, the therapeutic oil blend comprises alpha-pinene. In some embodiments, the therapeutic oil blend comprises 0.05-10% w/w alpha-pinene. In some embodiments, the therapeutic oil blend comprises 0.05-0.5% w/w alpha-pinene. In some embodiments, the therapeutic oil blend comprises 0.1-1% w/w alpha-pinene. In some embodiments, the therapeutic oil blend comprises 1-5% w/w alpha-pinene. In some embodiments, the therapeutic oil blend comprises 5-10% w/w alpha-pinene. In some embodiments, the therapeutic oil blend comprises at least 0.05%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1.0%, 1.5%, 2.0%, 2.5%, 3.0%, 3.5%, 4.0%, 4.5%, 5.5%, 6.0%, 6.5%, 7.0%, 7.5%, 8.0%, 8.5%, 9.0%, 9.5%, or 10.0% w/w alpha-pinene. In some embodiments, the therapeutic oil blend comprises about 0.05%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1.0%, 1.5%, 2.0%, 2.5%, 3.0%, 3.5%, 4.0%, 4.5%, 5.5%, 6.0%, 6.5%, 7.0%, 7.5%, 8.0%, 8.5%, 9.0%, 9.5%, or 10.0% w/w alpha-pinene. In some embodiments, the therapeutic oil blend comprises at most 0.05%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1.0%, 1.5%, 2.0%, 2.5%, 3.0%, 3.5%, 4.0%, 4.5%, 5.5%, 6.0%, 6.5%, 7.0%, 7.5%, 8.0%, 8.5%, 9.0%, 9.5%, or 10.0% w/w alpha-pinene.

In some embodiments, the therapeutic oil blend comprises beta-pinene. In some embodiments, the therapeutic oil blend comprises 0.05-10% w/w beta-pinene. In some embodiments, the therapeutic oil blend comprises 0.05-0.5% w/w beta-pinene. In some embodiments, the therapeutic oil blend comprises 0.1-1% w/w beta-pinene. In some embodiments, the therapeutic oil blend comprises 1-5% w/w beta-pinene. In some embodiments, the therapeutic oil blend comprises 5-10% w/w beta-pinene. In some embodiments, the therapeutic oil blend comprises at least 0.05%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1.0%, 1.5%, 2.0%, 2.5%, 3.0%, 3.5%, 4.0%, 4.5%, 5.5%, 6.0%, 6.5%, 7.0%, 7.5%, 8.0%, 8.5%, 9.0%, 9.5%, or 10.0% w/w beta-pinene. In some embodiments, the therapeutic oil blend comprises about 0.05%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1.0%, 1.5%, 2.0%, 2.5%, 3.0%, 3.5%, 4.0%, 4.5%, 5.5%, 6.0%, 6.5%, 7.0%, 7.5%, 8.0%, 8.5%, 9.0%, 9.5%, or 10.0% w/w beta-pinene. In some embodiments, the therapeutic oil blend comprises at most 0.05%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1.0%, 1.5%, 2.0%, 2.5%, 3.0%, 3.5%, 4.0%, 4.5%, 5.5%, 6.0%, 6.5%, 7.0%, 7.5%, 8.0%, 8.5%, 9.0%, 9.5%, or 10.0% w/w beta-pinene.

In some embodiments, the therapeutic oil blend comprises alpha-terpinene. In some embodiments, the therapeutic oil blend comprises 0.01-10% w/w alpha-terpinene. In some embodiments, the therapeutic oil blend comprises 0.01-0.05% w/w alpha-terpinene. In some embodiments, the therapeutic oil blend comprises 0.05-0.1% w/w alpha-terpinene. In some embodiments, the therapeutic oil blend comprises 0.1-0.5% w/w alpha-terpinene. In some embodiments, the therapeutic oil blend comprises 0.5-1% w/w alpha-terpinene. In some embodiments, the therapeutic oil blend comprises 1-5% w/w alpha-terpinene. In some embodiments, the therapeutic oil blend comprises 5-10% w/w alpha-terpinene. In some embodiments, the therapeutic oil blend comprises at least 0.01%, 0.02%, 0.03%, 0.04%, 0.05%, 0.06%, 0.07%, 0.08%, 0.09%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1.0%, 1.5%, 2.0%, 2.5%, 3.0%, 3.5%, 4.0%, 4.5%, 5.5%, 6.0%, 6.5%, 7.0%, 7.5%, 8.0%, 8.5%, 9.0%, 9.5%, or 10.0% w/w alpha-terpinene. In some embodiments, the therapeutic oil blend comprises about 0.01%, 0.02%, 0.03%, 0.04%, 0.05%, 0.06%, 0.07%, 0.08%, 0.09%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1.0%, 1.5%, 2.0%, 2.5%, 3.0%, 3.5%, 4.0%, 4.5%, 5.5%, 6.0%, 6.5%, 7.0%, 7.5%, 8.0%, 8.5%, 9.0%, 9.5%, or 10.0% w/w alpha-terpinene. In some embodiments, the therapeutic oil blend comprises at most 0.01%, 0.02%, 0.03%, 0.04%, 0.05%, 0.06%, 0.07%, 0.08%, 0.09%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1.0%, 1.5%, 2.0%, 2.5%, 3.0%, 3.5%, 4.0%, 4.5%, 5.5%, 6.0%, 6.5%, 7.0%, 7.5%, 8.0%, 8.5%, 9.0%, 9.5%, or 10.0% w/w alpha-terpinene.

In some embodiments, the therapeutic oil blend comprises gamma-terpinene. In some embodiments, the therapeutic oil blend comprises 0.01-10% w/w gamma-terpinene. In some embodiments, the therapeutic oil blend comprises 0.01-0.05% w/w gamma-terpinene. In some embodiments, the therapeutic oil blend comprises 0.05-0.1% w/w gamma-terpinene. In some embodiments, the therapeutic oil blend comprises 0.1-0.5% w/w gamma-terpinene. In some embodiments, the therapeutic oil blend comprises 0.5-1% w/w gamma-terpinene. In some embodiments, the therapeutic oil blend comprises 1-5% w/w gamma-terpinene. In some embodiments, the therapeutic oil blend comprises 5-10% w/w gamma-terpinene. In some embodiments, the therapeutic oil blend comprises at least 0.01%, 0.02%, 0.03%, 0.04%, 0.05%, 0.06%, 0.07%, 0.08%, 0.09%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1.0%, 1.5%, 2.0%, 2.5%, 3.0%, 3.5%, 4.0%, 4.5%, 5.5%, 6.0%, 6.5%, 7.0%, 7.5%, 8.0%, 8.5%, 9.0%, 9.5%, or 10.0% w/w gamma-terpinene. In some embodiments, the therapeutic oil blend comprises about 0.01%, 0.02%, 0.03%, 0.04%, 0.05%, 0.06%, 0.07%, 0.08%, 0.09%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1.0%, 1.5%, 2.0%, 2.5%, 3.0%, 3.5%, 4.0%, 4.5%, 5.5%, 6.0%, 6.5%, 7.0%, 7.5%, 8.0%, 8.5%, 9.0%, 9.5%, or 10.0% w/w gamma-terpinene. In some embodiments, the therapeutic oil blend comprises at most 0.01%, 0.02%, 0.03%, 0.04%, 0.05%, 0.06%, 0.07%, 0.08%, 0.09%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1.0%, 1.5%, 2.0%, 2.5%, 3.0%, 3.5%, 4.0%, 4.5%, 5.5%, 6.0%, 6.5%, 7.0%, 7.5%, 8.0%, 8.5%, 9.0%, 9.5%, or 10.0% w/w gamma-terpinene.

In some embodiments, the therapeutic oil of the present disclosure comprises between 38%-76% CBD oil, between 22%-44% clove oil, and between 9%-19% peppermint oil. In some embodiments, the therapeutic oil of the present disclosure comprises about 55% CBD oil, about 31% clove oil, and about 13.5% peppermint oil. In some embodiments, the therapeutic oil of the present disclosure comprises about 50% CBD.

In some embodiments, the therapeutic oil of the present disclosure comprises between 25%-50% CBD, between 0.9%-19% camphor, between 4.3% to 8.74% menthol, between 3.79% to 7.59% menthone, between 0.02% to 0.03% eucalyptol, between 0.15% to 0.30% a-pinene, between 0.15% to 0.31% b-pinene, between 0.38% to 0.76% b-myrcene, between 0.08% to 0.16% a-terpinene, between 1.25% to 2.5% d-limonene, between 0.08% to 0.16% y-terpinene, and between 0.07% to 0.14% linalool. In some embodiments, the therapeutic oil of the present disclosure comprises about 50% CBD, about 0.14% camphor, about 6.25% menthol, about 5.42% menthone, about 0.02% eucalyptol, about 0.21% a-pinene, about 0.22% b-pinene, about 0.54% b-myrcene, about 0.11% a-terpinene, about 1.78% d-limonene, about 0.11% y-terpinene, and about 0.1% linalool.

### Compositions Comprising Therapeutic Oil Blend

The present disclosure also provides compositions comprising the therapeutic oil blend of the disclosure. The compositions may comprise additional ingredients suited to their intended purpose and mode of administration. Such ingredients may include additional essential oils (that are added after the therapeutic oil is made), carrier oils, emulsifiers, humectants, flavoring, coloring, and the like. The compositions include, but are not limited to, topical compositions and ingestible compositions, including food compositions, tablets, tinctures, lotions, body balms, massage oils, drink mixes, gel topicals, salves, and lozenges. In this section the composition comprising the therapeutic oil blends are sometimes referred to as therapeutic oil blend compositions.

### Essential Oils

The compositions of the present disclosure can comprise one or more essential oils or essential oil compounds. Essential oils can include, but are not limited to: Linalool; B-Caryophyllene; B-Myrcene; D-Limonene; Humulene; a-Pinene; Ylang Ylang (Cananga odorata); Yarrow (Achillea millefolium); Violet (Viola odorata); Vetiver (Vetiveria zizanoides); Vanilla (Vanilla plantifolia); Tuberose (Polianthes tuberosa); Thyme (Thymus vulgaris L.); Tea Tree (Melaleuca alternifolia); Tangerine (Citrus reticulata); Spruce, Black (Picea mariana); Spruce (Tsuga Canadensis); Spikenard (Nardostachys jatamansi); Spearmint (Mentha spicata); Sandalwood (Santalum spicatum); Rosewood (Aniba rosaeodora); Rosemary Verbenone (Rosmarinus officinalis); Rosemary (Rosmarinus officinalis); Rose (Rosa damascena); Rose Geranium (Pelargonium roseum); Ravensara (Ravensara aromatica); Plai (Zingiber cassumunar) Pine Needle (Pinus sylvestris L.); Petitgrain (Citrus aurantium); Peppermint (Mentha piperita); Pepper, Black (Piper nigrum L.); Patchouli (Pogostemon cablin); Palo Santo (Bursera graveolens); Palmarosa (Cymbopogon martini); Osmanthus (Osmanthus fragrans); Oregano (Origanum vulgare); Orange, Sweet (Citrus sinensis); Oak Moss (Evernia prunastri); Nutmeg (Myristica fragrans) Niaouli (Melaleuca viridifloria); Neroli (aka Orange Blossom) (Citrus aurantium); Myrtle (Myrtus communis); Myrrh (Commiphora myrrha); Mimosa (Acacia decurrens); Melissa (Melissa officinalis L.); Marjoram, Sweet (Origanum majorana); Manuka (Leptospermum scoparium); Mandarin, Red (Citrus deliciosa); Mandarin (Citrus deliciosa); Lotus, White (Nelumbo nucifera); Lotus, Pink (Nelumbo nucifera); Lotus, Blue (Nelumbo nucifera); Lime (Citrus aurantifolia); Lily (Lilum aurantum); Lemongrass (Cymbopogon citratus); Lemon (Citrus limonum); Lavender (Lavandula angustifolium); Lavandin (Lavandula hybrida grosso); Kanuka (Kunzea ericoides); Juniper Berry (Juniperus cummunis); Jasmine (Jasminum officinale); Jasmine Abs (Jasminum sambac); Helichrysum (Helichrysum italicum); Grapefruit, White (Citrus×paradisi); Grapefruit, Pink (Citrus paradisi); Ginger (Zingiber officinalis); Geranium (Pelargonium graveolens); Geranium, Bourbon (Pelargonium graveolens, ‘Herit); Gardenia (Gardenia jasminoides); Galbanum (Ferula galbaniflua); Frankincense (Boswellia carterii); Frangipani (Plumeria alba); Fir Needle White (Abies alba); Fir Needle Siberia (Abies siberica); Fir Needle Canada (Abies balsamea); Fennel, Sweet (Foeniculum vulgare); Eucalyptus Smithii. Eucalyptus Radiata, Eucalyptus Globulus, Eucalyptus Citriodora, Eucalyptus Blue Mallee (Eucalyptus polybractea); Elemi (Canarium luzonicum); Dill (Anethum graveolens); Cypress (Cupressus sempervirens); Cumin (Cuminum cyminum); Coriander (Coriandum sativum); Cocoa (Theobroma cacao); Clove (Eugenia caryophylatta); Clary Sage (Salvia sclarea); Cistus (aka Labdanum) (Cistus ladaniferus L.); Cinnamon (Cinnamomum zeylanicum); Chamomile, Roman (Anthemis nobilis); Chamomile, Blue (Matricaria chamomilla); Celery Seed (Apium graveolins); Cedarwood, Western Red (Thuja plicata); Cedarwood, Blood (Juniperus virginiana); Cedarwood Atlas (Cedrus atlantica); Carrot Seed (Daucus carota); Cardamon (Elettaria cardamomum); Caraway Seed (Carum carvi); Cajeput (Melaleuca cajuputi); Cade (Juniperus oxycedrus); Birch, White (Betula alba); Birch, Sweet (Betula lenta); Bergamot (Citrus bergamia); Bay Laurel (Laurus nobilis); Basil (Ocimum basilicum); Basil, Holy (Ocimum sanctum); Basil (Ocimum basilicum); Balsam Poplar (Populus balsamifera); Balsam Peru (Myroxylon balsamum); Angelica (Angelica archangelica L.); and/or combinations thereof.

In some embodiments, the composition comprising the therapeutic oil blend of the present disclosure may comprise an essential oil derived from a plant selected from the following list: Alfalfa (Medicago sativa L.); Allspice (Pimenta officinalis Lindl.); Almont, bitter (pure from prussic acid) (Prunnus amygdalus Batsch, Prussun armeniaca L., or Prunnus persica (L.) Batsch.); Ambrette (seed) (Hibiscus moschatus Moench.); Angelica root (Angelica archangelica L.); Angelica seed; Angelica stem; Angostura (cusparia bark) (Galipea officinalis Hancock.); Anise (Pimpinella anisum L.); Asafetida (Ferula assa-foetida L. and related spp. of Ferula.); Balm (lemon balm) (Melissa officinalis L.); Balsam of Peru (Myroxylon pereirae Klotzsch.); Basil (Ocimum basilicum L.); Bay leaves (Laurus nobilis L.); Bay (myrcia oil) (Pimenta racemosa (Mill.) J. W. Moore.); Bergamot (bergamot orange) (Citrus aurantium L. subsp. bergamia Wright et Am.); Bitter almond (free from prussic acid) (Prunus amygdalus Batsch, Prunus armeniaca L., or Prunus persica (L.) Batsch.); Bois de rose (Aniba rosaeodora Ducke.); Cacao (Theobroma cacao L.); Camomile (chamomile) flowers, Hungarian (Matricaria chamomilla L.); Camomile (chamomile) flowers, Roman or English (Anthemis nobilis L.); Cananga (Cananga odorata Hook. f and Thoms.); Capsicum (Capsicum frutescens L. and Capsicum annuum L.); Caraway (Carum carvi L.); Cardamom seed (cardamon) (Elettaria cardamomum Maton.); Carob bean (Ceratonia siliqua L.); Carrot (Daucus carota L.); Cascarilla bark (Croton eluteria Benn.); Cassia bark, Chinese (Cinnamomum cassia Blume.); Cassia bark, Padang or Batavia (Cinnamomum burmanni Blume.); Cassia bark, Saigon (Cinnamomum loureirii Nees.); Celery seed (Apium graveolens L.); Cherry, wild, bark (Prunus serotina Ehrh.); Chervil (Anthriscus cerefolium (L.) Hoffm.); Chicory (Cichorium intybus L.); Cinnamon bark, Ceylon (Cinnamomum zeylanicum Nees.); Cinnamon bark, Chinese (Cinnamomum cassia Blume.); Cinnamon bark, Saigon (Cinnamomum loureirii Nees.); Cinnamon leaf, Ceylon (Cinnamomum zeylanicum Nees.); Cinnamon leaf, Chinese (Cinnamomum cassia Blume.); Cinnamon leaf, Saigon (Cinnamomum loureirii Nees.); Citronella (Cymbopogon nardus Rendle.); Citrus peels (Citrus spp.); Clary (clary sage) (Salvia sclarea L.); Clover (Trifolium spp.); Coca (decocainized) (Erythroxylum coca Lam. and other spp. of Erythroxylum.); Coffee (Coffea spp.); Cola nut (Cola acuminata Schott and Endl., and other spp. of Cola.); Coriander (Coriandrum sativum L.); Cumin (cummin) (Cuminum cyminum L.); Curacao orange peel (orange, bitter peel) (Citrus aurantium L.); Cusparia bark (Galipea officinalis Hancock.); Dandelion (Taraxacum officinale Weber and T. laevigatum DC.); Dandelion root; Dog grass (quackgrass, triticum) (Agropyron repens (L.) Beauv.); Elder flowers (Sambucus canadensis L. and S. nigra I.); Estragole (esdragol, esdragon, tarragon) (Artemisia dracunculus L.); Estragon (tarragon); Fennel, sweet (Foeniculum vulgare Mill.); Fenugreek (Trigonella foenum-graecum L.); Galanga (galangal) (Alpinia officinarum Hance.); Geranium (Pelargonium spp.); Geranium, East Indian (Cymbopogon martini Stapf.); Geranium, rose (Pelargonium graveolens L′Her.); Ginger (Zingiber officinale Rosc.); Grapefruit (Citrus paradisi Macf.); Guava (Psidium spp.); Hickory bark (Carya spp.); Horehound (hoarhound) (Marrubium vulgare L.); Hops (Humulus lupulus L.); Horsemint (Monarda punctata L.); Hyssop (Hyssopus officinalis L.); Immortelle (Helichrysum augustifolium DC.); Jasmine (Jasminum officinale L. and other spp. of Jasminum.); Juniper (berries) (Juniperus communis L.); Kola nut (Cola acuminata Schott and Endl., and other spp. of Cola.); Laurel berries (Laurus nobilis L.); Laurel leaves (Laurus spp.); Lavender (Lavandula officinalis Chaix.); Lavender, spike (Lavandula latifolia Vill.); Lavandin (Hybrids between Lavandula officinalis Chaix and Lavandula latifolin Vill.); Lemon (Citrus limon (L.) Burm. f.); Lemon balm (see balm); Lemon grass (Cymbopogon citratus DC. and Cymbopogon lexuosus Stapf.); Lemon peel (Citrus limon (L.) Burm. f.); Lime (Citrus aurantifolia Swingle.); Linden flowers (Tilia spp.); Locust bean (Ceratonia siliqua L,); Lupulin (Humulus lupulus L.); Mace (Myristica fragrans Houtt.); Mandarin (Citrus reticulata Blanco.); Marjoram, sweet (Majorana hortensis Moench.); Mate (Ilex paraguariensis St. Hil.); Melissa (see balm); Menthol (Mentha spp.); Menthyl acetate; Molasses (extract) (Saccarum officinarum L.); Mustard (Brassica spp.); Naringin (Citrus paradisi Macf.); Neroli, bigarade (Citrus aurantium L.); Nutmeg (Myristica fragrans Houtt.); Onion (Allium cepa L.); Orange, bitter, flowers (Citrus aurantium L.); Orange, bitter, peel; Orange leaf (Citrus sinensis (L.) Osbeck.); Orange, sweet; Orange, sweet, flowers; Orange, sweet, peel; Origanum (Origanum spp.); Palmarosa (Cymbopogon martini Stapf.); Paprika (Capsicum annuum L.); Parsley (Petroselinum crispum (Mill.) Mansf.); Pepper, black (Piper nigrum L.); Pepper, white; Peppermint (Mentha piperita L.); Peruvian balsam (Myroxylon pereirae Klotzsch.); Petitgrain (Citrus aurantium L.); Petitgrain lemon (Citrus limon (L.) Burm. f.); Petitgrain mandarin or tangerine (Citrus reticulata Blanco.); Pimenta (Pimenta officinalis Lindl.); Pimenta leaf (Pimenta officinalis Lindl.); Pipsissewa leaves (Chimaphila umbellata Nutt.); Pomegranate (Punica granatum L.); Prickly ash bark (Xanthoxylum (or Zanthoxylum) Americanum Mill. or Xanthoxylum clava-herculis L.); Rose absolute (Rosa alba L., Rosa centifolia L., Rosa damascena Mill., Rosa gallica L., and vars. of these spp.); Rose (otto of roses, attar of roses); Rose buds; Rose flowers; Rose fruit (hips); Rose geranium (Pelargonium graveolens L′Her.); Rose leaves (Rosa spp.); Rosemary (Rosmarinus officinalis L.); Saffron (Crocus sativus L.); Sage (Salvia officinalis L.); Sage, Greek (Salvia triloba L.); Sage, Spanish (Salvia lavandulaefolia Vahl.); St. John's bread (Ceratonia siliqua L.); Savory, summer (Satureia hortensis L.); Savory, winter (Satureia montana L.); Schinus molle (Schinus molle L.); Sloe berries (blackthorn berries) (Prunus spinosa L.); Spearmint (Mentha spicata L.); Spike lavender (Lavandula latifolia Vill.); Tamarind (Tamarindus indica L.); Tangerine (Citrus reticulata Blanco.); Tarragon (Artemisia dracunculus L.); Tea (Thea sinensis L.); Thyme (Thymus vulgaris L. and Thymus zygis var. gracilis Boiss.); Thyme, white; Thyme, wild or creeping (Thymus serpyllum L.); Triticum (see dog grass); Tuberose (Polianthes tuberosa L.); Turmeric (Curcuma longa L.); Vanilla (Vanilla planifolia Andr. or Vanilla tahitensis J. W. Moore.); Violet flowers (Viola odorata L.); Violet leaves; Violet leaves absolute; Wild cherry bark (Prunus serotina Ehrh.); Ylang-ylang (Cananga odorata Hook. f. and Thoms.); and Zedoary bark (Curcuma zedoaria Rosc.).

### Carrier Oils

In some embodiments, a composition comprising a therapeutic oil blend according to the present disclosure may also comprise a carrier oil. In some embodiments, the carrier oil may be any vegetable oil. In some embodiments, the carrier oil may be any essential oil. Various exemplary carrier oils and essential oils are described, for example, in WO 2017/091764 and any of these oils may be used in the therapeutic oil blend compositions of the present disclosure.

In some embodiments, the carrier oil may be medium chain triglyceride (MCT) oil, long chain triglyceride (LCT) oil, coconut oil, corn oil, canola oil, olive oil, avocado oil, vegetable oil, flaxseed oil, palm oil, peppermint oil, hemp oil, sesame oil, sunflower oil, a winterized oil of long-chain mono-, di-, and tri-glycerides (e.g. Maisine® CC), rice bran oil, or any combination thereof. Additional examples of carrier oils include, but are not limited to, almond oil; aloe vera oil; apricot kernel oil; avocado oil; argan oil; calendula oil; carrot seed oil; castor oil; coconut oil; evening primrose oil; fish oils and oils rich in omega-3 fatty acids (e.g., algae, krill, flaxseed); grape seed oil; hazelnut oil; hemp seed oil; jojoba oil; macadamia oil; olive oil; raspberry seed oil; sesame oil; sunflower oil; walnut oil; wheatgerm oil, and combinations thereof.

A carrier oil may be present in the therapeutic oil blend or the composition comprising the therapeutic oil blend in an amount ranging from about 0.5% to about 99.5%. In an embodiment, a carrier oil may be present in the therapeutic oil blend composition in an amount of about 5% (w/w), about 10% (w/w), about 15% (w/w), about 20% (w/w), about 25% (w/w), about 30% (w/w), about 35% (w/w), about 40% (w/w), about 45% (w/w), about 50% (w/w), about 55% (w/w), about 60% (w/w), about 65% (w/w), about 70% (w/w), about 75% (w/w), about 80% (w/w), about 85% (w/w), about 90% (w/w), about 95% (w/w), about 96% (w/w), about 97% (w/w), about 98% (w/w), about 99% (w/w), or more.

### Emulsifiers

In some embodiments, the composition comprises about 5% to about 25% by weight of an emulsifier or a mixture of emulsifiers. In some embodiments, the composition comprises about 25% to about 50% by weight of an emulsifier or a mixture of emulsifiers. In some embodiments, the emulsifier is selected from the group consisting of polysorbate 80, oleoyl polyoxyl-6 glycerides, polyoxyl 35 hydrogenated castor oil, sucrose distearate, tocopherol polyethylene glycol 1000 succinate, lauroyl polyoxyl-32 glycerides, sorbitan monooleate, glyceryl stearate, cetearyl alcohol, sodium stearoyl lactylate, salts thereof, derivatives thereof, and mixtures of emulsifiers.

In some embodiments, the cannabinoid oil compositions of the present disclosure comprise an emulsifier. Emulsifier components may be selected from the group consisting of poly-glycolized glycerides and polyoxyethylene glycerides of medium to long chain mono-, di-, and triglycerides, such as: almond oil PEG-6 esters, almond oil PEG-60 esters, apricot kernel oil PEG-6 esters (Labrafil® M1944CS), caprylic/capric triglycerides PEG-4 esters (Labrafac® Hydro WL 1219), caprylic/capric triglycerides PEG-4 complex (Labrafac® Hydrophile), caprylic/capric glycerides PEG-6 esters (Softigen® 767), caprylic/capric glycerides PEG-8 esters (Labrasol®), castor oil PEG-50 esters, hydrogenated castor oil PEG-5 esters, hydrogenated castor oil PEG-7 esters, 9 hydrogenated castor oil PEG-9 esters, corn oil PEG-6 esters (Labrafil® M 2125 CS), corn oil PEG-8 esters (Labrafil® WL 2609 BS), corn glycerides PEG-60 esters, olive oil PEG-6 esters (Labrafil® M1980 CS), hydrogenated palm/palm kernel oil PEG-6 esters (Labrafil® M 2130 BS), hydrogenated palm/palm kernel oil PEG-6 esters with palm kernel oil, PEG-6, palm oil (Labrafil® M 2130 CS), palm kernel oil PEG-40 esters, peanut oil PEG-6 esters (Labrafil® M 1969 CS), glycerol esters of saturated C8-C18 fatty acids (Gelucire® 33/01), glyceryl esters of saturated C12-C18 fatty acids (Gelucire® 39/01 and 43/01), glyceryl laurate/PEG-32 laurate (Gelucire® 44/14), glyceryl laurate glyceryl/PEG 20 laurate, glyceryl laurate glyceryl/PEG 32 laurate, glyceryl, laurate glyceryl/PEG 40 laurate, glyceryl oleate/PEG-20 glyceryl, glyceryl oleate/PEG-30 oleate, glyceryl palmitostearate/PEG-32 palmitostearate (Gelucire® 50/13), glyceryl stearate/PEG stearate, glyceryl stearate/PEG-32 stearate (Gelucire® 53/10), saturated polyglycolized glycerides (Gelucire® 37/02 and Gelucire® 50/02), triisostearin PEG-6 esters (i.e. Labrafil® Isostearique), triolein PEG-6 esters, trioleate PEG-25 esters, polyoxyl 35 castor oil (Cremophor® EL or Kolliphor® EL), polyoxyl 40 hydrogenated castor oil (Cremophor® RH 40 or Kolliphor® RH40), polyoxyl 60 hydrogenated castor oil (Cremophor® RH60), lecithin, phospholipids and mixtures thereof.

In some embodiments, the emulsifier is polyglycolized derivatives and polyoxyethylene esters or ethers derivatives of medium to long chain fatty acids, commercially named Brij and Myrj variety surfactants, and propylene glycol esters of medium to long chain fatty acids, which can be used including caprylate/caprate diglycerides, glyceryl monooleate, glyceryl ricinoleate, glyceryl laurate, glyceryl dilaurate, glyceryl dioleate, glyceryl mono/dioleate, glyceryl caprylate/caprate, medium chain (C8/C10) mono- and diglycerides (Capmul® MCM, Capmul® MCM (L)), mono- and diacetylated monoglycerides, polyglyceryl oleate, polyglyceryl-2 dioleate, polyglyceryl-10 trioleate, polyglyceryl-10 laurate, polyglyceryl-10 oleate, and polyglyceryl-10 mono dioleate, propylene glycol caprylate/caprate (Labrafac® PC), propylene glycol dicaprylate/dicaprate (Miglyol® 840), propylene glycol monolaurate, propylene glycol ricinoleate, propylene glycol monooleate, propylene glycol dicaprylate/dicaprate, propylene glycol dioctanoate, and mixtures thereof.

### Humectants

In some embodiments, the therapeutic oil blend composition is a topical formulation and further comprises a humectant, which can be referred to as a soothing, smoothing, moisturizing or protective agent. The humectants in, e.g., a lotion composition functions to stabilize the moisture content of the tissue to which it is applied in the presence of fluctuating humidity. The humectant is not particularly limited, as numerous acceptable humectants are known in the art. Examples of suitable humectants for use in the compositions of the present disclosure include: polyglycols (as hereinafter defined), propylene glycol, sorbitol, lactic acid, sodium lactate, glycerol, ethoxylated castor oil, calamine, dodecylsulphate, sodium lauryl sulphate (SLS); a polyoxyethylene ester of polysorbitan, such as monooleate, monolaurate, monopalmitate, monostearate esters; esters of sorbitan, the polyoxyethylenes ethers, sodium dioctylsulphosuccinate (DOSS), lecithin, sodium docusate, hexylene glycol, butylene glycol, aloe vera gel, aloe vera powder, hyaluronic acid, alpha hydroxy acids such as lactic acid, egg yolk, egg white, glyceryl triacetate, honey, molasses, polymeric polyols such as polydextrose, quillaia, sodium hexametaphosphate e452i; sugar alcohols (sugar polyols) such as glycerol, sorbitol, xylitol, maltitol; urea, and castor oil. The amount of the humectant in a topical formulation is not particularly limited, so long as it is a therapeutically effective amount. In some embodiments, the amount ranges from 0.01 to 5 wt %, relative to the total amount of the composition. In some embodiments, the amount ranges from 0.1 to 1 wt %, relative to the total amount of the composition.

### Probiotics

Probiotics can also be included in cannabinoid oil compositions prepared according to the present disclosure. Examples of suitable probiotics include, but are not limited to, Acinetobacter calcoaceticus, Arthrobacter agilis, Arthrobacter citreus, Arthrobacter globiformis, Arthrobacter luteus, Arthrobacter simplex, Azotobacter chroococcum, Azotobacter paspali, Azospirillum brasiliense, Azospirillum lipoferum, Bacillus ssp. (e.g., Bacillus brevis, Bacillus coagulans, Bacillus laterosporus, Bacillus marcerans, Bacillus pumilus, Bacillus polymyxa, Bacillus sphaericus, Bacillus subtilis), Bacteroides lipolyticum, Bacteroides succinogenes, Bifidobacterium ssp. (e.g., Bifidobacterium animalis lactis, Bifidobacterium bifidum, Bifidobacterium infantis, Bifidobacterium lactis, Bifidobacterium longum, Bifidobacterium animalis, Bifidobacterium breve), Brevibacterium lipolyticum, Brevibacterium stationis, Enterococcus faecium, Kurthia zopfii, Lactobacillus ssp. (e.g., Lactobacillus acidophilus, Lactobacillus brevis, Lactobacillus bulgaricus, Lactobacillus casei, Lactobacillus delbrueckii LE, Lactobacillus ferment, Lactobacillus gasseri, Lactobacillus helveticus, Lactobacillus lacris, Lactobacillus paracasei, Lactobacillus plantarumtarum, Lactobacillus reuteri, Lactobacillus rhamnosus, Lactobacillus salivarius, Lactobacillus sporogenes), Myrothecium verrucaria, Pseudomonas calcis, Pseudomonas dentrificans, Pseudomonas fluorescens, Pseudomonas glathei, Phanerochaete chrysosporium, Saccharomyces boulardii, Saccharomyces cerevisiae, Streptococcus thermophilus, Streptomyces fradiae, Streptomyces cellulosae, Streptomyces griseoflavus, and combinations thereof.

### Additional Ingredients

The compositions of the present disclosure can comprise an additional agent or agents, whether active or passive. Examples of such an agent include a sweetening agent, a flavoring agent, a coloring agent, a filling agent, a binding agent, a lubricating agent, an excipient, a preservative, an emollient, a hydrating agent, a smoothing agent, or a manufacturing agent. Additional pharmaceutically acceptable excipients (in the case of pharmaceuticals) or other additives (for non-pharmaceutical applications) can be added to the composition. For example, if desired, any generally accepted soluble or insoluble inert pharmaceutical filler (diluent) material can be included in the final product (e.g., a solid dosage form). Such inert pharmaceutical filler can comprise a monosaccharide, a disaccharide, a polyhydric alcohol, inorganic phosphates, sulfates or carbonates, and combinations thereof. Examples of suitable inert pharmaceutical fillers include sucrose, dextrose, lactose, xylitol, fructose, sorbitol, calcium phosphate, calcium sulfate, calcium carbonate, microcrystalline cellulose, and combinations thereof. An effective amount of any generally accepted pharmaceutical lubricant, such as calcium or magnesium soaps, can be added.

Depending on the dosage form, optional additives and modifiers further comprise one or more of acids, bases, acidity regulators, alcohol, anticaking agents, antifoaming agents, antioxidants, bulking agents, coagulation agents, colour retention agents, emulsifiers, flavor enhancers, flour treatment agents, gelling agents, glazing agents, humectants, leavening agents, tracer gases, preservatives, stabilizers, sweeteners, tenderizers, and thickeners.

Additional herbal extracts, essential oils, homeopathic remedies, and flower essences are known and may also be added to the therapeutic oil blend compositions of the present disclosure. Examples of such are provided in U.S. Publication No. 20180344661, incorporated herein by reference in its entirety. The therapeutic oil blend composition may also comprise a fruit extract, such as an extract of coconuts, apricots, apples, pears, peaches, pineapples, papayas, pomegranates, cherries, kiwis, tangerines, oranges, grapes, or mixtures thereof. Additional fruit extracts and additives may be found in, e.g., U.S. Pat. No. 6,630,163, incorporate by reference herein in its entirety.

Other ingredients may also be present in the composition, such as antibiotics; antiseptics; antifungals; corticosteroids; soothing agents; anti-aging agents; smoothing agents; moisturizing agents; and protective agents.

### Topical Formulations

In some embodiments, the composition comprising the therapeutic oil blend is a topical formulation. Without wishing to be bound by theory, it is believed that the smaller particle size, increased bioavailability, and increased shelf life of the therapeutic oil blend of the present disclosure makes it well suited for use and administration via topical formulation. The form of the topical formulations of the present invention is not particularly limited, if it is in a form that promotes its use as a topical formulation. Non-limiting examples of the formulation include a lotion, a cream, a salve, a body balm, a liniment, an ointment, a gel, a paste, a tonic, an unguent, a nasal spray, a soap, a shampoo, and a lip balm.

In some embodiments, a composition comprising the therapeutic oil blend of the present disclosure is a lotion. Unless otherwise indicated, the term “lotion” relates to a low- to medium-viscosity topical preparation intended for application to unbroken skin in contrast, creams and gels have higher viscosity. Lotions are applied to external skin with bare hands, a clean cloth, cotton wool or gauze. Many lotions, especially hand lotions and body lotions are formulated not as a medicine delivery system, but simply to smooth, re-hydrate, and soften the skin. These are particularly popular with the aging and aged demographic groups, in the case of face usage, can also be classified as a cosmetic in many cases, and may contain fragrances.

Most lotions are oil-in-water emulsions using a substance such as cetyl alcohol to keep the emulsion together, but water-in-oil lotions are also formulated. The key components of a skin care lotion, cream or gel emulsion (that is mixtures of oil and water) are the aqueous and oil phases, an emulsifier to prevent separation of these two phases, and, if used, the drug substance or substances. Other ingredients are commonly added to lotions, such as fragrances, glycerol, petroleum jelly, dyes, preservatives, proteins and stabilizing agents.

In some embodiments, the lotion comprises the therapeutic oil blend of the present disclosure. In some embodiments, the lotion comprises a therapeutic oil blend of the present disclosure comprising cannabidiol or a cannabidiol-related cannabinoid, a terpene, an emulsifier, a carrier oil, and an essential oil. In some embodiments, the lotion comprises arnica oil. Arnica oil may be used for relieving pain associated with sore muscles, muscle aches, sprains, back and neck pain. Arnica oil may be beneficial for bruise treatment, insect bites, soothing sunburn, and to reduce inflammation in joints. In some embodiments, the lotion comprises rose absolute oil. Rose absolute oil may be used to treat pain, anxiety, and depression. Rose absolute oil may have calming and/or sedative effects.

In some embodiments, the lotion comprises a carrier oil that is a plant-derived oil. In some embodiments, the lotion comprises one or more of the following plant-derived oils: almond oil, avocado seed oil, beech nut oil, bitter gourd oil, bottle gourd oil, brazil nut oil, buffalo gourd oil, butternut squash seed oil, canola oil, cashew oil, cocoa butter, coconut oil, corn oil, cottonseed oil, egusi seed oil, flax seed oil, grapefruit seed oil, grapeseed oil, hazelnut oil, hemp oil, lemon oil, macadamia oil, mongongo nut oil, olive oil, orange oil, palm oil, peanut oil, pecan oil, pine nut oil, pistachio oil, pumpkin seed oil, rapeseed oil, rice bran oil, safflower seed oil, sesame seed oil, sunflower seed oil, soybean oil, walnut oil, watermelon seed oil. In some embodiments, the lotion may comprise olive oil, sunflower oil, and/or hempseed oil.

Lotions can be used for the delivery to the skin of medications such as: antibiotics; antiseptics; antifungals; corticosteroids; anti-acne agents; anti-inflammatory agents; anti-arthritis agents; anti-aging agents; soothing agents; anti-wrinkle agents; smoothing agents; moisturizing agents; and protective agents.

In some embodiments, the composition is a body butter, a body balm, or a salve. In general, body butters and body balms are made utilizing the base ingredients of natural butter(s) and herbal and/or vegetable/nut/seed oils. Balms typically contain beeswax. Salves typically comprise herbal oils and beeswax. Since body balms, butters, and salves do not contain water, they are may be referred to as anhydrous formulations. The lack of water also means they do not require a preservative. In some embodiments, an antioxidant may be added, such as turmeric CO₂ oil, rosemary CO₂ extract, vitamin E or mixed tocopherols.

In some embodiments, a composition comprising the therapeutic oil blend of the present disclosure is a body balm. In some embodiments, the body balm may comprise one or more ingredients selected from the list consisting of: shea butter, beeswax, coconut oil, sunflower oil, jojoba oil, cocoa butter, MCT oil, turmeric oil, rosemary extract, vitamin E, and essential oils. In some embodiments, the body balm comprises turmeric oil. In some embodiments, the turmeric oil is CO₂ extracted turmeric oil. In some embodiments, the body balm has antioxidant effects. In some embodiments, the body balm has anti-inflammatory effects.

In some embodiments, a composition comprising the therapeutic oil blend of the present disclosure is a salve. In some embodiments, the salve comprises a carrier oil, such as a vegetable oil. In some embodiments, the salve comprises olive oil, sunflower oil, or coconut oil. In some embodiments, the salve comprises herbal extracts and/or oils. In some embodiments, the salve comprises an essential oil. In some embodiments, the salve comprises beeswax.

In some embodiments, a composition comprising the therapeutic oil blend of the present disclosure is a massage oil. In some embodiments, the therapeutic oil blend itself may be used as a massage oil. In some embodiments, the massage oil comprises a carrier oil, such as those disclosed herein. In some embodiments, the massage oil comprises a carrier oil selected from the list consisting of: sunflower oil, jojoba oil, sesame seed oil, olive oil, mineral oil, sweet almond oil, apricot oil, or grapeseed oil. In some embodiments, the massage oil comprises an essential oil. In some embodiments, the massage oil comprises a vitamin or mineral.

In some embodiments, a composition comprising the therapeutic oil blend of the present disclosure is a topical gel. A gel is a solid jelly-like soft material that comprises a substantially dilute cross-linked system, which exhibits no flow when in the steady-state. By weight, gels are mostly liquid, yet they behave like solids due to a three-dimensional cross-linked network within the liquid. It is the crosslinking within the fluid that gives a gel its structure (hardness) and contributes to the adhesive stick (tack). Gels are a dispersion of molecules of a liquid within a solid medium. In some embodiments, the gel comprises a crosslinking agent. In some embodiments, the gel comprises carbomer.

Cannabinoids can be formulated in a product, such as a topical product, in a quantity of at least about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, or 2000 milligrams (mg), or more, per ounce (oz). Cannabinoids can be formulated in a product, such as a topical product, in a quantity of at most about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, or 2000 milligrams (mg) per ounce (oz). Cannabinoids can be formulated in a product, such as a topical product, in a quantity of about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, or 2000 milligrams (mg) per ounce (oz). Cannabinoids can be formulated in a product, such as a topical product, in a quantity of at least about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50% or more by weight of the product. Cannabinoids can be formulated in a product, such as a topical product, in a quantity of at most about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of the product. Cannabinoids can be formulated in a product, such as a topical product, in a quantity of about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of the product.

The therapeutic oil blends described herein comprise terpenes. The terpene content of the therapeutic oil blend is described in detail in the foregoing disclosure. In addition to the terpenes comprised by the therapeutic oil blend, additional terpene compounds can be included in a product, such as a topical product, in a quantity of at least about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, or 2000 milligrams (mg), or more, per ounce (oz). Terpene compounds can be included in a product, such as a topical product, in a quantity of at most about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, or 2000 milligrams (mg) per ounce (oz). Terpene compounds can be included in a product, such as a topical product, in a quantity of about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, or 2000 milligrams (mg) per ounce. Terpene compounds can be included in a product, such as a topical product, in a quantity of at least about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, or more by weight of the product. Terpene compounds can be included in a product, such as a topical product, in a quantity of at most about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of the product. Terpene compounds can be included in a product, such as a topical product, in a quantity of about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of the product.

### Ingestible Formulations

In some embodiments, the composition comprising a therapeutic oil blend according to the present disclosure is an ingestible formulation. Without wishing to be bound by theory, it is believed that the smaller particle size, increased bioavailability, and increased shelf life of the therapeutic oil blend of the present disclosure makes it well suited for use and administration vian ingestible formulation. The form of the ingestible formulations of the present disclosure is not particularly limited, if it is in a form that promotes its use as an ingestible formulation. Non-limiting examples of the formulation include a tablet, a tincture, a drink mix, a beverage, a lozenge, and a food composition.

In some embodiments, the composition comprising the therapeutic oil blend of the present disclosure is a tablet. In some embodiments, the tablet is a pressed tablet formulated to dissolve in an aqueous medium, e.g., for use as a drink mix. In some embodiments, the composition can take the form of a tablet or a capsule prepared by conventional means with a pharmaceutically acceptable excipient. Tablets and gelatin capsules may comprise any number of base components, including: diluents or fillers, lubricants, binders, disintegrants, and wetting agents.

Exemplary embodiments of diluents and fillers include, without limitation, lactose, dextrose, sucrose, mannitol, maltodextrin, lecithin, agarose, xanthan gum, guar gum, glycerol, sorbitol, cellulose (e.g., ethyl cellulose, microcrystalline cellulose), glycine, pectin, polyacrylates and/or calcium hydrogen phosphate and calcium sulfate. Exemplary embodiments of lubricants include, without limitation, silica, anhydrous colloidal silica, talcum, stearic acid, its magnesium or calcium salt (e.g., magnesium stearate or calcium stearate), metallic stearates, colloidal silicon dioxide, hydrogenated vegetable oil, corn starch, sodium benzoate, sodium acetate and/or polyethyleneglycol. Exemplary embodiments of binders include, without limitation, magnesium aluminum silicate, starch paste, gelatin, tragacanth, methylcellulose, sodium carboxymethylcellulose, polyvinylpyrrolidone and/or hydroxypropyl methylcellulose. Exemplary embodiments of disintegrants include, without limitation, starches (e.g., potato starch or sodium starch), glycolate, agar, alginic acid or its sodium or potassium salt, or effervescent mixtures. Exemplary embodiments of wetting agents include, without limitation, sodium lauryl sulfate. Excipients may also be used for preparation of buccal dosage forms and sublingual dosage forms (e.g., lozenges), for example, in U.S. Pat. Nos. 5,981,552 and 8,475,832.

Tablets can be formulated in sustained release format. Methods of making sustained release tablets are known in the art; see, for example, U.S. Patent Publication No. 2006/0051416 and U.S. Patent Publication No. 2007/0065512. Gradual-release tablets are known in the art; examples of such tablets are set forth in U.S. Pat. No. 3,456,049, for example. A slow- or sustained-release form may delay disintegration or absorption of the composition or one or more components thereof.

In some embodiments, the tablet comprises essential oils. In some embodiments, the tablet comprises ingredients useful in promoting alertness. In some embodiments, the tablet comprises ingredients useful in promoting somnolence and/or enhancing sleep quality.

In some embodiments, the composition comprising the therapeutic oil blend of the present disclosure is a tincture. A tincture is typically an extract of plant or animal material dissolved in a solvent or carrier liquid. In some embodiments, solvent or carrier liquid concentrations may be between 15-60% v/v. In some embodiments, solvent concentrations may be at most 10%, at most 20%, at most 30%, at most 40%, at most 50%, at most 60%, at most 70%, at most 80%, or at most 90% v/v. The solvent or carrier liquid may be selected from an alcohol, vinegar, glycerol, propylene glycol, and a carrier oil. In some embodiments, the tincture comprises a carrier oil. In some embodiments, the solvents, through the process of solvation, create solvation spheres around the cannabinoids. In some embodiments, the tincture comprises essential oils. In some embodiments, the tincture comprises additional ingredients, such as sweeteners and/or flavoring. In some embodiments, the tincture is formulated with ingredients, e.g., essential oils, useful in promoting somnolence and/or improving sleep quality. In some embodiments, the tincture is formulated with ingredients, e.g., essential oils, useful in decreasing or relieving or ameliorating stress or symptoms of stress.

In some embodiments, the composition comprising the therapeutic oil blend of the present disclosure is a lozenge. In some embodiments, the lozenge comprises a syrup. In some embodiments, the lozenge comprises isomalt. In some embodiments, the lozenge comprises food coloring. In some embodiments, the lozenge comprises flavoring. In some embodiments, the lozenge comprises a sweetening agent. In some embodiments, the lozenge comprises essential oils.

In some embodiments, the composition comprising the therapeutic oil blend of the present disclosure is a food composition. The food composition comprises an effective amount of a therapeutic oil blend and a food carrier. Any type, amount, or form of composition comprising a therapeutic oil blend disclosed herein is applicable for preparing the food composition disclosed herein.

A food composition or food product can comprise a food bar, including but not limited to granola bars, protein bars, candy bars, and energy bars. A food composition or food product can comprise a cereal product, including but not limited to oatmeal, flour (e.g., wheat flour, rice flour, corn flour, barley flour), breakfast cereal, granola, bread, pasta, rice cakes, and popcorn. A food composition or food product can comprise a bakery product, including but not limited to bread, pastries, brownies, cakes, pies, donuts, crackers, and muffins. A food composition or food product can comprise a dairy product, including but not limited to milk, fermented milk, curd, whey, yogurt, cream, cheese, butter, clarified butter, ghee, and ice cream. A food composition or food product can comprise a nut butter or seed butter, including but not limited to peanut butter, almond butter, cashew butter, hazelnut butter, macadamia nut butter, pecan butter, pistachio butter, walnut butter, pumpkin seed butter, sesame seed butter, soybean butter, and sunflower seed butter. A food composition or food product can comprise an oil (e.g., a cooking oil), including but not limited to olive oil, coconut oil, vegetable oil, canola oil, corn oil, peanut oil, sunflower seed oil, almond oil, avocado oil, rice bran oil, cottonseed oil, flaxseed oil, linseed oil, grape seed oil, hemp oil, mustard oil, macadamia oil, palm oil, tea seed oil, walnut oil, margarine, lard, butter, clarified butter, ghee, or tallow. A food composition or food product can comprise sports food products such as energy gels, sports drinks, energy powders, energy bars, energy shots, protein powders, drink mixes, and protein drinks (e.g., protein shakes). A food composition or food product can comprise a beverage, including but not limited to water, electrolyte drinks, soda, coconut water, tea (e.g., Jun tea, black tea, green tea, white tea, herbal tea), coffee, a soft drink, an alcoholic beverage (e.g., cocktail, liquor, spirits, beer, wine, malt beverage), water, juice (e.g., apple juice, orange juice, tomato juice, vegetable juice, cranberry juice), a sports drink, electrolyte-enriched water, vitamin-enhanced water, a hangover-recovery drink, milk (e.g., dairy-based milk, coconut milk, almond milk, soy milk, hemp milk, rice milk, oat milk, cashew milk, hazelnut milk), and yogurt. A food composition or food product can comprise a fungus or fermented food or drink, including but not limited to kifir (kefir), jun, amasi, amazake, appam, ayran, doogh, bagoong, brem, cheonggukj ang, chicha, kombucha, fermented bean curd, kimchi, lassi, miso, poi, yakult, and yogurt.

Nutritional additives and modifiers useful to the consumer may also be included in the therapeutic oil blend compositions of the present disclose. Nutritional additives function in the maintenance of normal body health. Suitably nutritional additives comprise essential nutrients including vitamins, dietary minerals amino acids and fatty acids, including without limitation vitamin A, vitamin B1, vitamin B2, vitamin B3, vitamin B5, vitamin B6, vitamin B7, vitamin B9, vitamin B12, vitamin C, vitamin D, vitamin E, vitamin K, calcium, phosphorus, potassium, sulfur, sodium, chlorine, magnesium, iron, cobalt, copper, zinc, molybdenum, iodine, selenium, manganese, nickel, chromium, fluorine, boron, strontium histidine, isoleucine, leucine, lysine, methionine, cysteine, phenylalanine, tyrosine, threonine, tryptophan, valine, alpha-linoleic acid, and linoleic acid.

Cannabinoids can be formulated in a product, such as an ingestible product, in a quantity of at least about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, or 2000 milligrams (mg), or more, per ounce (oz). Cannabinoids can be formulated in a product, such as an ingestible product, in a quantity of at most about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, or 2000 milligrams (mg) per ounce (oz). Cannabinoids can be formulated in a product, such as an ingestible product, in a quantity of about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, or 2000 milligrams (mg) per ounce (oz). Cannabinoids can be formulated in a product, such as an ingestible product, in a quantity of at least about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50% or more by weight of the product. Cannabinoids can be formulated in a product, such as an ingestible product, in a quantity of at most about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of the product. Cannabinoids can be formulated in a product, such as an ingestible product, in a quantity of about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of the product.

Terpene compounds can be included in a product, such as an ingestible product, in a quantity of at least about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, or 2000 milligrams (mg), or more, per ounce (oz). Terpene compounds can be included in a product, such as an ingestible product, in a quantity of at most about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, or 2000 milligrams (mg) per ounce (oz). Terpene compounds can be included in a product, such as an ingestible product, in a quantity of about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, or 2000 milligrams (mg) per ounce. Terpene compounds can be included in a product, such as an ingestible product, in a quantity of at least about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, or more by weight of the product. Terpene compounds can be included in a product, such as an ingestible product, in a quantity of at most about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of the product. Terpene compounds can be included in a product, such as an ingestible product, in a quantity of about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of the product.

### Organic Products

Embodiments of the present disclosure are directed to compositions comprising at least 95% organic ingredients. In the United States, the Agricultural Marketing Service of the U.S. Department of Agriculture (USDA) oversees the National Organic Program (NOP). The NOP regulations include a definition of “organic” and provide for certification that agricultural ingredients have been produced under conditions that would meet the definition. They also include labeling standards based on the percentage of organic ingredients in a product.

The NOP applies the term “Organic” to products containing at least 95 percent organically produced ingredients (excluding water and salt). Remaining product ingredients must consist of nonagricultural substances approved on the National List or non-organically produced agricultural products that are not commercially available in organic form. See ams.usda.gov/rules-regulations/organic/national-list). Products meeting these requirements may display the USDA Organic Seal and must display the certifying agent's name and address.

In some embodiments, present compositions comprise greater than 95% organic components. In some embodiments, other ingredients suitable for inclusion in the compositions of the disclosure are listed in The National List of Allowed and Prohibited Substances, which may be found at ams.usda.gov/rules-regulations/organic/national-list. Also found in this reference are materials not intended for inclusion in the compositions herein. The list is incorporated by reference herein in its entirety.

### Methods of Use of Therapeutic Oil Blends and Compositions Comprising

The therapeutic oil blends and compositions comprising (collectively “the compositions”) of the present disclosure may be used in the treatment of numerous conditions. In some embodiments, the compositions may be administered to improve one or more physical or psychological attributes associated with a condition.

In some embodiments, the compositions may be administered to improve one or more physical or psychological attributes in the absence of a medical condition or unrelated to a medical condition. For example, the compositions may be administered to improve alertness, sleep quality, sense of wellbeing, recovery after physical exertion, skin quality, muscle tension, or any of a number of physical and psychological attributes.

Subjects of the present disclosure can include humans and other animals, such as pets (e.g., dogs, cats, birds, small mammals, snakes) and livestock or farm animals (e.g., cows, pigs, horses, sheep, chickens). Compositions of the present disclosure can be useful for both human and veterinary applications.

### Target Diseases and Conditions

In some embodiments, any of the compositions described above is for use in a method of treating a cannabinoid-responsive symptom, disease or disorder. The phrase “cannabinoid-responsive symptom, disease or disorder” as used herein refers to any symptom, disease or disorder which is associated with therapeutic benefit by a cannabinoid, by a mixture of cannabinoids, or by extracts of Cannabis.

In some embodiments, the cannabinoid-responsive symptom, disease or disorder is selected from the group consisting of: pain associated with cancer, neuropathic pain and HIV-associated sensory neuropathy; side effects of chemotherapy including nausea; symptoms of neurology and neurodegenerative diseases such as Huntington's disease, Parkinson's disease, Alzheimer's disease, amyotrophic lateral sclerosis, multiple sclerosis, epilepsy, post-traumatic stress disorder (PTSD), alcohol abuse, bipolar disorder, depression, anorexia nervosa; cancer such as gliomas, leukemia, skin tumors, colorectal cancer; diseases including hepatitis C, methicillin-resistant Staphylococcus aureus (MRSA), pruritus, psoriasis, asthma, sickle-cell disease, sleep apnea, digestive diseases, collagen-induced arthritis, atherosclerosis and dystonia and geriatric syndromes. Each possibility represents a separate embodiment of the invention. Additional indications are those that are treated by pure cannabinoids or benefiting from high dose of cannabinoids, for example, graft versus host disease (GVHD), inflammatory bowel diseases (IBD), psychiatric indications such as schizophrenia and borderline personality disorders (BPD), and opioid addiction withdrawal syndrome. In an embodiment, the pharmaceutical composition is used to treat psychiatric disorders, borderline personality disorder, anxiety, impulsivity, instability, suicidal, self-injury, or anhedonia behavior.

In some embodiments, the compositions may be used in the treatment of pain. Examples of pain include muscle pain, neuropathic pain, neurogenic pain, back pain, migraine, headache, facial pain, endometriosis, neuropathic pain associated with post-herpetic neuralgia, diabetic neuropathy, post-episiotomy pain, joint pain, musculoskeletal pain, trigeminal neuralgia, chronic pain, and the like. The compositions of the present disclosure may be used to mediate pain perception and inflammation, and indicate potential in the topical symptomatic treatment of the following conditions: neuropathic pain, burning feet syndrome, diabetic foot neuropathy, chronic low back pain, fibromyalgia syndrome, neck pain, post herpetic neuralgia, pain and inflammation of arthritis (hands, knees, joints); rheumatoid arthritis and osteoarthritis, trigeminal neuralgia (TN), pudendal neuropathy and pudendal nerve entrapment (PNE), sciatica pain, muscle strains, tendon injuries, plantar fasciitis, herniated disks; phantom limb pain following amputation, and dermatological disorders like psoriasis, eczema, dermatitis.

In some embodiments, the compositions may be used to treat a disease related to oxidative stress. In some embodiments, the disease is neurodegenerative disease, inflammation, metabolic disorders, aging, cancer, and atherosclerosis. cancer, aging, inflammation, malaria, rheumatoid arthritis, neurodegenerative diseases, diabetes, hypertension, chronic obstructive pulmonary disease, angina, arrhythmia, asthma, benign prostatic hyperplasia, carpal tunnel syndrome, bipolar disorder, cancer, cardiovascular disease, cataracts, Celiac disease, chronic fatigue syndrome, congestive heart failure, Crohn's disease, depression, dermatitis, diabetes, erectile dysfunction, fibromyalgia, gastroesophageal reflux disease, glaucoma, hypercholesterolemia, influenza, kidney stones, Lyme disease, macular degeneration, psoriasis, sleep apnea, systemic lupus erythematosus, thrombosis, and tinnitus. In some embodiments, administering the composition improves the one or more health conditions.

In some embodiments, a composition of the present disclosure may be administered to a subject suffering from one or more health conditions. Non-limiting examples of various health conditions include cancer, aging, inflammation, malaria, rheumatoid arthritis, neurodegenerative diseases, diabetes, hypertension, chronic obstructive pulmonary disease, angina, arrhythmia, asthma, benign prostatic hyperplasia, carpal tunnel syndrome, bipolar disorder, cancer, cardiovascular disease, cataracts, Celiac disease, chronic fatigue syndrome, congestive heart failure, Crohn's disease, depression, dermatitis, diabetes, erectile dysfunction, migraine, fibromyalgia, gastroesophageal reflux disease, glaucoma, hypercholesterolemia, influenza, kidney stones, Lyme disease, macular degeneration, psoriasis, sleep apnea, systemic lupus erythematosus, thrombosis, and tinnitus.

In some embodiments, a composition of the present disclosure may be used irrespective of the presence or absence of a medical condition. In some embodiments, a composition may be used to improve one or more aspects of daily life. In some embodiments, a composition may be used for the treatment of stress, for improving mood, for increasing relaxation, or for promoting a sense of well-being. In some embodiments, a composition may be used to improve one or more aspects of the waking and sleeping cycles. For example, a composition, e.g., a tincture, may be used to improve one or more aspects of sleep, including an aspect selected from sleep duration, restlessness, nighttime waking, restfulness, time to fall asleep, time spent asleep, time spent in deep sleep, time spent in REM sleep, time spent in light sleep, and overall quality of sleep. In some embodiments, a composition may be used in a waking subject to improve alertness, mood, activity, concentration, stress, focus, or wakefulness.

In some embodiments, a composition of the present disclosure, e.g., a topical formulation, may be used to improve one or more aspects of skin health, quality, or maintenance. For example, a composition may be used to improve skin hydration, appearance, dryness, tautness, tension, oiliness, texture, or smoothness.

In some embodiments, a composition of the present disclosure may be used to improve one or more aspects of physical health related to routine physical fitness. For example, a composition may be used to improve muscle regeneration, muscular discomfort, joint discomfort, soreness, muscle aches, muscle strain, or muscle exhaustion related to physical activity or exercise. In some embodiments, a composition may be applied (in the case of a topical formulation) or consumed (in the case of an oral formulation) following a period of physical exertion to improve one or more aspects of the recovery experience, including but not limited to recovery duration, comfort, stiffness, soreness, and muscle/joint pain.

### Administration

For the purposes of administration, the present compositions may be formulated in a variety of dosage forms. The term “dosage form” denotes any form of the formulation that contains an amount of a cannabinoid or of a mixture of cannabinoids and a terpene or a mixture of terpenes sufficient to achieve at least a partial therapeutic effect with a single or repeat administration. In some embodiments, the dosage form is a topical dosage form. In some embodiments, the dosage form is an oral dosage form. In some embodiments, the dosage form is a nasal dosage form, rectal dosage form, mucosal dosage form, vaginal dosage form, or ear dosage form. In some embodiments, the dosage form is one of those described in the foregoing sections, e.g., an oil, a massage oil, a lotion, a gel, a salve, a body balm, a tincture, a tablet, a pressed tablet, a drink mix, a food substance, and the like.

Compositions can be formulated in forms including but not limited to liquid, gel, semi-solid, and solid. Compositions disclosed herein can further be processed into forms including but not limited to solids, powders, liquids, suspensions, gels, tablets, foods, lotions, cosmetics, and other forms discussed in this disclosure. Compositions can be provided in, for example, a tablet form, a capsule form, a food form a chewable form, a non-chewable form, a transbuccal form, a sublingual form, a slow-release form, a non-slow-release form, a sustained release form, or a non-sustained-release form.

In some embodiments, the dosage form is formulated as a hard shell capsule, a soft shell capsule, a tablet, a liquid, a syrup or enema or pessaries or ovule. In some embodiments, the dosage form is granules or pellets delivered in a sachet or filled into capsule or compressed into a tablet. Each possibility represents a separate embodiment of the invention. In some embodiments the dosage form is formulated for oral delivery or topical delivery. In some embodiments, the dosage form is formulated for mucosal delivery. In some embodiments, the dosage form is formulated as or in a candy, toffee, dragee, chocolate, cookie or lozenge.

Encapsulated dosage forms and methods for the manufacture thereof may be found in U.S. Patent Publication No. 20190247325, herein incorporated by reference in its entirety.

Cannabinoids, terpenes and flavonoids tend may be lipophilic and/or have low solubility in hydrophilic biocompatible matrix materials. One method for obtaining desirable dosage forms comprising lipophilic substances and hydrophilic biocompatible matrix substances is to encapsulate or disperse lipophilic substances in the hydrophilic matrix using additives or modifiers which provide an environment for stable oil-in-water emulsions, micelles, liposomes or other complex phase equilibrium modified compositions. Exemplary techniques, modifiers and additives are described, e.g., in U.S. Publication No. 20190321330, incorporated by reference herein in its entirety.

In some embodiments, the dosage form comprises at least about 50 mg of a cannabinoid or a mixture thereof. In some embodiments, the dosage form comprises at least about 100 mg of a cannabinoid or a mixture thereof. In some embodiments, the dosage form comprises at least about 200 mg of a cannabinoid or a mixture thereof. In some embodiments, the dosage form comprises at least about 300 mg of a cannabinoid or a mixture thereof. In some embodiments, the dosage form comprises at least about 400 mg of a cannabinoid or a mixture thereof. In some embodiments, the dosage form comprises about 50 mg to about 1,200 mg of a cannabinoid or a mixture thereof. In some embodiments, the dosage form comprises about 50 mg to about 500 mg of a cannabinoid or a mixture thereof. In some embodiments, the dosage form comprises about 100 mg to about 400 mg of a cannabinoid or a mixture thereof. In some embodiments, the dosage form comprises about 100 mg, about 200 mg, about 300 mg, about 400 mg, about 500 mg or about 600 mg of a cannabinoid or a mixture thereof. Each possibility represents a separate embodiment of the invention.

In some embodiments, the CBD compositions of the present disclosure may be administered topically or orally. For topical or oral administration, the dosage may be distributed throughout the composition in a concentration range of 50 mg to 1 g per ounce. In some embodiments, the dosage may be about 10, 50, 100, 150, 200, 250, 300, 350, 400, 450, 500, 550, 600, 650, 700, 750, 800, 850, 900, 950, or 1000 mg per ounce. In some embodiments, the dosage may be about 150 mg per ounce. In some embodiments, the dosage may be about 500 mg per ounce. In some embodiments, the dosage may be about 10, 50, 100, 150, 200, 250, 300, 350, 400, 450, 500, 550, 600, 650, 700, 750, 800, 850, 900, 950, or 1000 mg per individual dose.

Cannabidiol for treating various psychiatric indications may be prescribed at doses of 100 to 600 mg/day. Jalali and Johnson (International Neuropsychiatric Disease Journal, 2013, Vol. 1(2), pages 113-147) review numerous studies using THC and/or CBD for antipsychotic activity using 140 to 600 mg/day. Yeshurun and co-workers (Biol. Blood Marrow Transplant, 2015, Vol. 21(10), pages 1770-1775) successfully treated Graft-versus-host-disease (GVHD) using pure CBD at a dose of 600 mg/day. Devinsky and co-workers (Epilepsia, 2014, Vol. 55(6), pages 791-802) used 200 to 300 mg of CBD for treating anticonvulsant in epilepsy and other neurological disorders. Leweke and co-workers (Psychiatry, 2012, Vol. 15, pages 2012-2015) used up to 800 mg cannabidiol per day to alleviate psychotic symptoms of schizophrenia. Hurd and co-workers, (Neurotherapeutics, 2015, pages 607-15) used 400 and 800 mg cannabidiol as treatment for opioid addiction. Devinsky and co-workers, (The Lancet Neurology, 2016, Vol. 15(3) pages 270-278) treated patients with treatment-resistant epilepsy with median 23 mg/kg cannabidiol which is 500 mg to >1,000 mg per day for many patients. Chagas and co-workers (Journal of Psychopharmacology, 2014, Vol 28(11), pages 1088-1092) treated Parkinson's disease with 300 mg of cannabidiol.

In some embodiments, an effective amount of a composition comprising a therapeutic oil blend is administered to a subject. The term “effective amount” or “therapeutically effective amount” refers to that amount of a cannabinoid composition described herein that is sufficient to effect the intended application including but not limited to a reduction in oxidative stress in a cell and/or disease treatment in a subject. The therapeutically effective amount may vary depending upon) the subject and condition being treated, e.g., the weight and age of the subject, the severity of the disease condition, the manner of administration and the like, which can readily be determined by one of ordinary skill in the art. The term also applies to a dose that will induce a particular response in target cells, e.g., reduction in oxidative stress, reduction in pain, anesthesia effect, analgesic effect, etc. The specific dose will vary depending on the particular formulation of the cannabinoid composition, the dosing regimen to be followed, whether it is administered in combination with other compounds, timing of administration, the tissue to which it is administered, route of administration and the physical delivery system in which it is carried.

Any of the subject compositions can be provided in a unit dosage form. A unit dosage is an amount of a compound, such as a cannabinoid compound delivered alone or in combination with other components, which is to be administered to a subject at or about one time point. Other components which can be included with a unit dosage include but are not limited to cosmetics, food carriers, food bars, baked goods, dairy products, oils, beverages, solid dosages (e.g., tablets), or liquid dosages. A unit dosage of a cannabinoid compound can be about 10, 20, 30, 40, 50, 60, 70, 80, 90, 100, 125, 150, 175, 200, 250, 300, 350, 400, 450, 500, 600, 700, 800, 900, 1000, 1100, 1200, 1300, 1400, 1500, 1600, 1700, 1800, 1900, 2000 or more milligrams (mg). A unit dosage of a cannabinoid compound can be at least about 10, 20, 30, 40, 50, 60, 70, 80, 90, 100, 125, 150, 175, 200, 250, 300, 350, 400, 450, 500, 600, 700, 800, 900, 1000, 1100, 1200, 1300, 1400, 1500, 1600, 1700, 1800, 1900, 2000 or more milligrams (mg). A unit dosage of a cannabinoid compound can be at most about 2000, 1900, 1800, 1700, 1600, 1500, 1400, 1300, 1200, 1100, 1000, 900, 800, 700, 600, 500, 450, 400, 350, 300, 250, 200, 175, 150, 125, 100, 90, 80, 70, 60, 50, 40, 30, 20, 10, or less milligrams (mg). A unit dosage can be an hourly dosage. A unit dosage can be a daily dosage. A unit dosage can provide about 1/24, 1/12, ⅛, ¼, ¼, ⅓, ½, or all of a daily dosage of one or more cannabinoids for a subject. A unit dosage can take the form of a tablet, gel, liquid, food product, food bar, container of liquid of defined volume, or other forms described herein, packaged for one-time consumption or administration.

The amount of the composition of the subject method administered will be dependent on the subject being treated, the severity of disorder or condition, the rate of administration, the disposition of the composition and encapsulated cannabinoid compound. An effective dosage is in the range of about 0.1 mg to about 2000 mg per kg body weight per day. For example, for a 70 kg human, this may amount to about 7 mg/day to about 1.75 g/day. In some instances, dosage levels below the lower limit of the aforesaid range may be more than adequate, while in other case still larger doses may be employed without causing any harmful side effects, e.g., by dividing such larger doses in several small doses for administration throughout the day. Dosages may be administered over time periods of hours, days, weeks, or months.

The composition comprising an encapsulated cannabinoid compound can be administered to a subject in any of several ways to effect a reduction in oxidative stress. The compositions can be administered, for example, orally or transdermally. Other delivery routes include but are not limited to intranasal, sublingual, transmucosal, or intradermal. An effective amount of the compositions may be in in dosage unit formulations containing conventional nontoxic physiologically acceptable carriers, adjuvants, and vehicles as desired.

Oral administration may be accomplished using an oral dosage form. Oral dosage forms can be solid (e.g., a tablet or bulk powder) or liquid (e.g., a suspension or slurry). Tablets can include tablets, pressed tablets, caplets, capsules, including soft gelatin capsules, and lozenges. Tablets can further comprise suitable binders, lubricants, diluents, disintegrating agents, colorants, flavoring agents, flow-inducing agents, and melting agents. Liquids can include drink mixes and tinctures.

Compositions formulated for oral administration can be incorporated into a food composition. A food composition can be a beverage, a solid food, or a semi-solid food. Food compositions can comprise an encapsulated cannabinoid compound and a food carrier. A food carrier can be practically any food product. Examples of food carriers include, but are not limited to food bars (granola bars, protein bars, candy bars, etc.), cereal products (oatmeal, breakfast cereals, granola, etc.), bakery products (bread, donuts, crackers, bagels, pastries, cakes, etc.), beverages (milk-based beverage, sports drinks, fruit juices, alcoholic beverages, bottled waters), pastas, grains (rice, corn, oats, rye, wheat, flour, etc.), egg products, snacks (candy, chips, gum, chocolate, etc.), meats, fruits, and vegetables.

A composition can also be administered transdermally, such as via a patch. The compositions of the present disclosure can be administered intravenously. The compositions of the present disclosure can be administered topically. The compositions of the present disclosure can be administered via topical exposure to an aqueous solution, such as immersing a subject in a float tank. The compositions of the present disclosure can be formulated as a bath salt or liquid bath product, which can be dissolved or dispersed in water (e.g., a bath) for skin exposure, for example by immersion of the subject. Compositions comprising an encapsulated cannabinoid compound for topical or transdermal application can be provided as cosmetics or personal care products, such as soaps (e.g., solid, bar, liquid, or foaming), hand sanitizer, lotions, massage oils masks, makeup, moisturizers, body balms, salves, topical gels, sunscreen, toothpaste, mouth wash, or throat spray.

In some embodiments, the composition is administered orally. In some embodiments, the composition is a food or beverage. In some embodiments, the therapeutic oil blend of the present disclosure may be administered topically. In some embodiments, the therapeutic oil blend of the present disclosure may be administered dermally. In some embodiments, the therapeutic oil blend of the present disclosure may be administered transdermally. In some embodiments, the composition is administered by inhalation. In some embodiments, the composition is nebulized.

The cannabis oil extracts described herein are useful in the manufacture of a pharmaceutical composition or a medicament for treating a number of conditions including, but not limited to, the conditions listed in the foregoing sections.

Pharmaceutical compositions or medicaments for use in the present invention can be formulated by standard techniques or methods well-known in the art of pharmacy using one or more physiologically acceptable carriers or excipients. Suitable pharmaceutical carriers are described herein and in, e.g., “Remington's Pharmaceutical Sciences” by E. W. Martin. Cannabis oil extracts can be formulated for administration by any suitable route, including, but not limited to, orally, topically, nasally, rectally, vaginally, pulmonary, parenterally (e.g., intravenously, subcutaneously, intramuscularly, etc.), and combinations thereof. In some embodiments, the cannabis oil is diluted in a liquid, e.g., a carrier oil. The most suitable route of administration in any given case will depend in part on the condition being treated as well as the response of the subject to the particular route of treatment.

In certain embodiments, cannabis oil compositions as described herein are administered via a vaporizer or like device as described, for example, in U.S. Pat. No. 8,915,254; U.S. Pat. Appl. Pub. No. 2014/0060552; U.S. Pat. No. 8,488,952; and U.S. Pat. Appl. Pub. No. 2015/0040926. Compositions for pulmonary administration also include, but are not limited to, dry powder compositions consisting of the powder of a cannabis oil described herein, and the powder of a suitable carrier and/or lubricant. The compositions for pulmonary administration can be inhaled from any suitable dry powder inhaler device known to a person skilled in the art. In certain instances, the compositions may be conveniently delivered in the form of an aerosol spray from pressurized packs or a nebulizer, with the use of a suitable propellant, for example, dichlorodifluoromethane, trichlorofluoromethane, dichlorotetrafluoroethane, carbon dioxide, or other suitable gas. In the case of a pressurized aerosol, the dosage unit can be determined by providing a valve to deliver a metered amount. Capsules and cartridges of, for example, gelatin for use in an inhaler or insufflator can be formulated containing a powder mix of the compound(s) and a suitable powder base, for example, lactose or starch.

For oral administration, a pharmaceutical composition or a medicament can take the form of, e.g., a tablet or a capsule prepared by conventional means with a pharmaceutically acceptable excipient. Preferred are tablets and gelatin capsules comprising the active ingredient(s), together with (a) diluents or fillers, e.g., lactose, dextrose, sucrose, mannitol, maltodextrin, lecithin, agarose, xanthan gum, guar gum, sorbitol, cellulose (e.g., ethyl cellulose, microcrystalline cellulose), glycine, pectin, polyacrylates and/or calcium hydrogen phosphate, calcium sulfate, (b) lubricants, e.g., silica, anhydrous colloidal silica, talcum, stearic acid, its magnesium or calcium salt (e.g., magnesium stearate or calcium stearate), metallic stearates, colloidal silicon dioxide, hydrogenated vegetable oil, corn starch, sodium benzoate, sodium acetate and/or polyethyleneglycol; for tablets also (c) binders, e.g., magnesium aluminum silicate, starch paste, gelatin, tragacanth, methylcellulose, sodium carboxymethylcellulose, polyvinylpyrrolidone and/or hydroxypropyl methylcellulose; if desired (d) disintegrants, e.g., starches (e.g., potato starch or sodium starch), glycolate, agar, alginic acid or its sodium or potassium salt, or effervescent mixtures; (e) wetting agents, e.g., sodium lauryl sulfate, and/or (f) absorbents, colorants, flavors and sweeteners. Tablets can be either uncoated or coated according to methods known in the art. The excipients described herein can also be used for preparation of buccal dosage forms and sublingual dosage forms (e.g., films and lozenges) as described, for example, in U.S. Pat. Nos. 5,981,552 and 8,475,832. Formulation in chewing gums as described, for example, in U.S. Pat. No. 8,722,022, is also contemplated.

Further preparations for oral administration can take the form of, for example, solutions, syrups, suspensions, and toothpastes. Liquid preparations for oral administration can be prepared by conventional means with pharmaceutically acceptable additives, for example, suspending agents, for example, sorbitol syrup, cellulose derivatives, or hydrogenated edible fats; emulsifying agents, for example, lecithin, xanthan gum, or acacia; non-aqueous vehicles, for example, almond oil, sesame oil, hemp seed oil, fish oil, oily esters, ethyl alcohol, or fractionated vegetable oils; and preservatives, for example, methyl or propyl-p-hydroxybenzoates or sorbic acid. The preparations can also contain buffer salts, flavoring, coloring, and/or sweetening agents as appropriate.

Typical formulations for topical administration include creams, ointments, sprays, lotions, hydrocolloid dressings, and patches, as well as eye drops, ear drops, and deodorants. Cannabis oils can be administered via transdermal patches as described, for example, in U.S. Pat. Appl. Pub. No. 2015/0126595 and U.S. Pat. No. 8,449,908. Formulation for rectal or vaginal administration is also contemplated. The cannabis oils can be formulated, for example, as suppositories containing conventional suppository bases such as cocoa butter and other glycerides as described in U.S. Pat. Nos. 5,508,037 and 4,933,363. Compositions can contain other solidifying agents such as shea butter, beeswax, kokum butter, mango butter, ilipe butter, tamanu butter, carnauba wax, emulsifying wax, soy wax, castor wax, rice bran wax, and candelila wax. Compositions can further include clays (e.g., Bentonite, French green clays, Fuller's earth, Rhassoul clay, white kaolin clay) and salts (e.g., sea salt, Himalayan pink salt, and magnesium salts such as Epsom salt).

The compositions set forth herein can be formulated for parenteral administration by injection, for example by bolus injection or continuous infusion. Formulations for injection can be presented in unit dosage form, for example, in ampoules or in multi-dose containers, optionally with an added preservative. Injectable compositions are preferably aqueous isotonic solutions or suspensions, and suppositories are preferably prepared from fatty emulsions or suspensions. The compositions may be sterilized and/or contain adjuvants, such as preserving, stabilizing, wetting or emulsifying agents, solution promoters, salts for regulating the osmotic pressure, buffers, and/or other ingredients. Alternatively, the compositions can be in powder form for reconstitution with a suitable vehicle, for example, a carrier oil, before use. In addition, the compositions may also contain other therapeutic agents or substances. The compositions are prepared according to conventional mixing, granulating or coating methods, respectively, and contain about 0.1 to 75%, preferably about 1 to 50%, of the cannabis oils.

The aspects and embodiments provided herein have been described in an illustrative manner, and it is to be understood that the terminology used is intended to be in the nature of words of description rather than of limitation.

Many modifications and variations are possible in light of the above teachings. It is, therefore, to be understood that within the scope of the appended claims, the invention can be practiced otherwise than as specifically described.

The present disclosure is illustrated in detail below with reference to examples, but is not to be construed as being limited thereto.

Citation of any document herein is not intended as an admission that such document is pertinent prior art, or considered material to the patentability of any claim of the present disclosure. Any statement as to content or a date of any document is based on the information available to applicant at the time of filing and does not constitute an admission as to the correctness of such a statement.

### EXAMPLES

### Example 1: Therapeutic Oil Blend Formulation Process Via Sonication

A therapeutic oil blend comprising a cannabidiol-related cannabinoid and a terpene was prepared using an ultrasonic homogenizer (1800 W, 19-22 kHz). The cannabidiol-related cannabinoid was provided in the form of a cannabidiol-related cannabinoid-containing hemp oil. The hemp oil was heated to 60° C. to fully liquefy the hemp oil and sonicated for two hours at maximum power in order to homogenize the solution and prepare it for the addition of terpenes. The sonication was performed by alternating between sonication and rest periods at every 10 seconds in order to control the temperature of the sample, keeping the temperature of the sample below 80° C. throughout the process. The terpene was chilled in an effort to prevent vaporization and to reduce temperature, and then was added to the sonicated hemp oil during a further sonication cycle of ten minutes alternating between sonication and rest time. The therapeutic oil blend was then cooled and set to run for two hours alternating between sonication and rest time to fully homogenize the mixture. The resulting therapeutic oil blend had an average cannabidiol-related cannabinoid particle size of 20-60 nm and was shelf stable without crystallization for a period of at least 30 days.

### Example 2: Therapeutic Oil Blend Formulation Process Via Sonication

### Materials

A therapeutic oil blend comprising cannabidiol (CBD), eugenol, menthol, menthone, and d-limonene was prepared using an ultrasonic homogenizer (1800 W, 19-22 kHz). The cannabidiol was provided in the form of a CBD-comprising hemp oil; eugenol was provided in the form of a clove bud oil comprising greater than 90% eugenol; and all other terpenes were provided in the form of a fractionated peppermint oil. Table 1 provides exemplary quantities of these three ingredients, including the quantities of Formula 3 employed in this example and other ratios also being pursued experimentally.

TABLE 1
Quantities of ingredients comprised by exemplary
therapeutic oil blends.

| | Formula | Formula | Formula | Formula | Formula |
|---|---|---|---|---|---|
| Ingredient | 1 | 2 | 3 | 4 | 5 |
| Hemp oil | 40 g | 50 g | 60 g | 60 g | 70 g |
| Clove bud oil | 35 g | 35 g | 30 g | 20 g | 15 g |
| Peppermint oil | 25 g | 15 g | 10 g | 20 g | 15 g |

### Methods

To begin with, the hemp oil was heated to 60° C. to ensure that all the cannabinoids were adequately dissolved. Heating ensures that any crystals that may have formed during the storage and transport of the oil are dissolved before beginning the formulation work.

Next, the liquid hemp oil was sonicated for two hours at maximum power to ensure complete homogeneity of the product. In order to avoid overheating, the sonication was performed via a series of alternating sonication and rest periods cycling every 10 seconds. Related experiments had shown that overheating of the CBD or CBD+ terpenes resulted in degradation of the ingredients, producing a dark and thick product that failed to stay in liquid form in storage. In this example, temperature was controlled via a series of on/off cycles. Persons having skill in the art however, will recognize other ways of maintaining temperature stability. After two hours of alternating on/off sonication, the oil was ready to have additional ingredients added.

Terpenes can be sensitive to high temperatures (e.g., can evaporate due to their high vapor pressure). Thus, in order to reduce any losses, two steps were taken to keep the temperature of the solution steady. First, subsequent sonication steps followed the alternating cycle of sonication and rest periods every 10 seconds. Second, additional ingredients to the composition were added after being chilled. Thus, chilled eugenol was added to the composition, and the mixture was further sonicated for ten minutes. Next, chilled d-limonene was added to the composition, and the mixture was further sonicated for about 15 minutes. Camphor, eucalyptol, a-pinene, b-pinene, b-myrcene, a-terpinene, y-terpinene, and linalool were mixed at room temperature, allowing any solid ingredients to dissolve, and added during another sonication cycle of 20 minutes alternating between sonication and rest time.

### Results

The resulting therapeutic oil blend was then cooled again and set to run for two hours alternating between sonication and rest time to ensure complete mixing. The resulting therapeutic oil blend exhibited a variety of desirable stability, bioavailability, and particle size properties that made it an excellent candidate for topical cannabinoid therapies. For example, the therapeutic oil blend had an average particle size of 20-60 nm, as it refers to the size of the CBD particles and/or crystal structures within the oil. In addition, the therapeutic oil blend was shelf stable without crystallization for a period of at least 30 days, time point limited only by observation length. In contrast, the control mixture of the ingredients without sonication crystallized in 24 hours, had a sticky texture, and did not absorb well into the skin. These properties are illustrated in more detail in the Examples below.

The cannabinoid profiles of two exemplary therapeutic oil blends prepared according to this process were examined and the results are shown in Table 2.

TABLE 2
Cannabinoid profile.

| Compound | Result (%) | Result (mg/g) |
|---|---|---|
| Cannabidiol (CBD) | 50-52 | 500-520 |
| Other cannabinoids | 3.2-5.0 | 33-50 |

### Example 3: Large Scale Production of an Exemplary Therapeutic Oil Blend of the Disclosure

Example 2, above, described small-scale production of therapeutic oil blends. This example describes large (industrial scale) production of an exemplary therapeutic oil blend comprising CBD, eugenol, and other terpenes.

### Materials

In this industrial-scale example, the CBD was provided via CBD-comprising hemp oil, and the eugenol ingredient was provided via clove bud oil, comprising greater than 90% eugenol. The other terpenes, including menthol, menthone, d-limonene, camphor, eucalyptol, a-pinene, b-pinene, b-myrcene, a-terpinene, y-terpinene, and linalool were provided in the form of peppermint oil. Thus, in total, only three ingredients were necessary. Table 3 provides quantities of these three ingredients being pursued experimentally for use in exemplary therapeutic oil blends of the disclosure, including those of Formula 4 that were used in this example.

TABLE 3
Quantities of ingredients comprised by exemplary
therapeutic oil blends.

| | Formula | Formula | Formula | Formula | Formula |
|---|---|---|---|---|---|
| Ingredient | 1 | 2 | 3 | 4 | 5 |
| Hemp oil | 50 kg | 60 kg | 60 kg | 70 kg | 80 kg |
| Clove bud oil | 45 kg | 40 kg | 30 kg | 30 kg | 25 kg |
| Peppermint oil | 15 kg | 10 kg | 20 kg | 10 kg | 5 kg |

### Methods

A process similar to that employed in Example 2 was carried out. Briefly, hemp oil was heated to 60° C. and sonicated for two hours at maximum power, alternating between sonication and rest periods at every 10 seconds. Next, chilled clove bud oil was added and the mixture was further sonicated for ten minutes alternating between sonication and rest time. Finally, chilled peppermint oil was added and the mixture was further sonicated for 20 minutes alternating between sonication and rest time. To ensure complete mixing, the therapeutic oil blend was then cooled and set to run for two hours alternating between sonication and rest time.

### Results

The resulting therapeutic oil blend, or an equivalent batch obtained by the same process, was shelf stable without crystallization for a period of at least 60 days, as demonstrated in Example 6 below.

### Example 4: Open Air Stability and Non-Crystallization Testing

Traditional hemp oils are known to crystallize over time, causing issues with oxidation and non-homogeneity. In particular, the formation of crystal structures allows for greater surface area exposure to air and increased oxidation rate. To demonstrate the improved stability and non-crystallizing properties of a therapeutic oil blend of the disclosure, different samples were tested for long term stability in open air testing conditions.

### Materials and Methods

Sample 1, hemp oil control. A full spectrum hemp oil enriched for CBD (Rocky Mountain Extraction Services, LLC) was weighed out and placed in a beaker. The sample was then heated to 60° C. and allowed to completely liquify for 5 minutes. It was then allowed to cool at room temperature and neutral humidity and the beaker was exposed to the open air. This sample served as a control for traditional, commercially available hemp oils without added terpenes and without sonication.

Sample 2, therapeutic oil blend. An exemplary therapeutic oil blend of the disclosure was formulated according to the process outlined in Example 3 above using the same weight of a full spectrum CBD hemp oil from the same lot used for Sample 1. During the formulation process, the sample reached 60° C. and was left to cool at room temperature and neutral humidity. It was then placed inside a beaker and exposed to the open air. Table 4 discloses the differences between these samples.

TABLE 4
Characteristics of samples employed in open air stability testing.

| Sample | Ingredients | Process |
|---|---|---|
| Sample 1, | CBD-containing | Heated to 60° C. |
| hemp oil | hemp oil | to liquefy. |
| Sample 2, | CBD-containing hemp | Heated, sonicated, further |
| therapeutic | oil, clove bud oil, | sonicated with serial |
| oil blend | peppermint oil | addition of clove bud oil |
| | | and peppermint oil as in Ex. 3 |

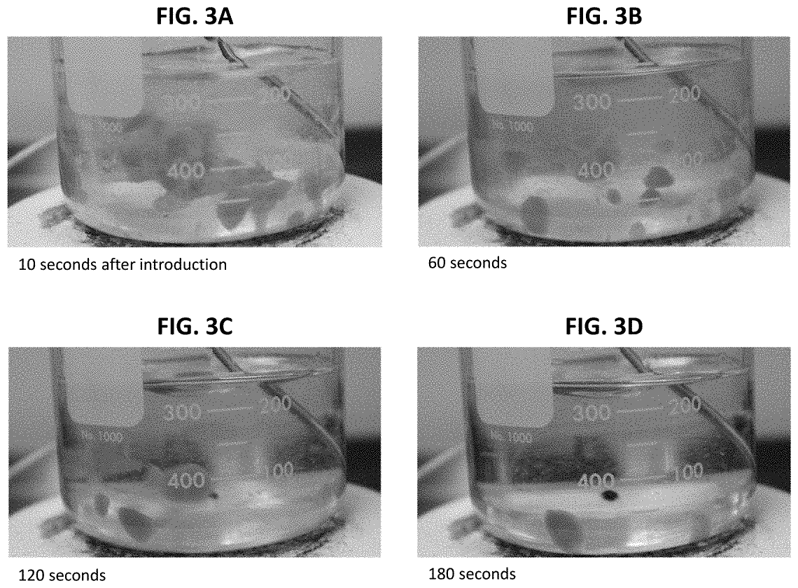

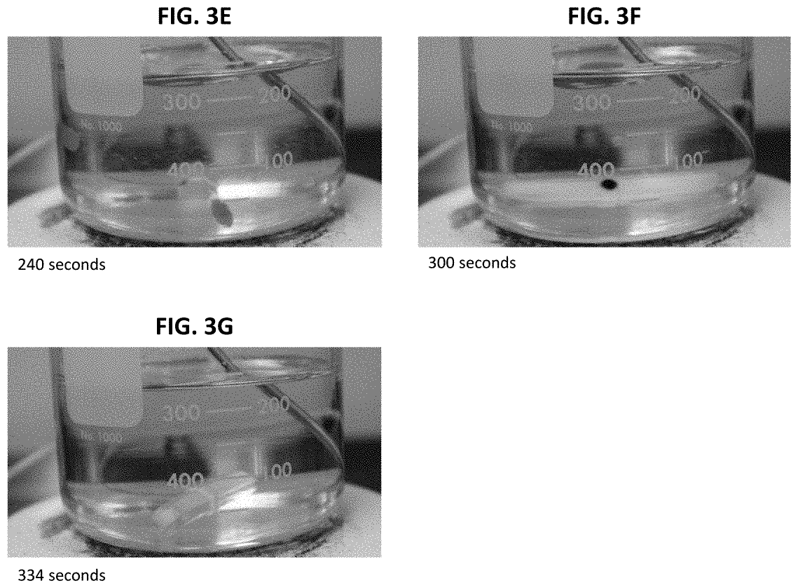

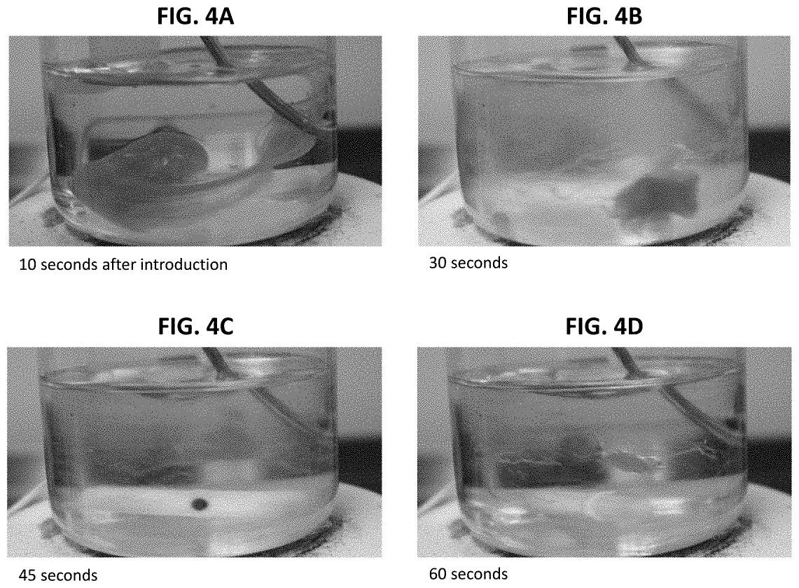

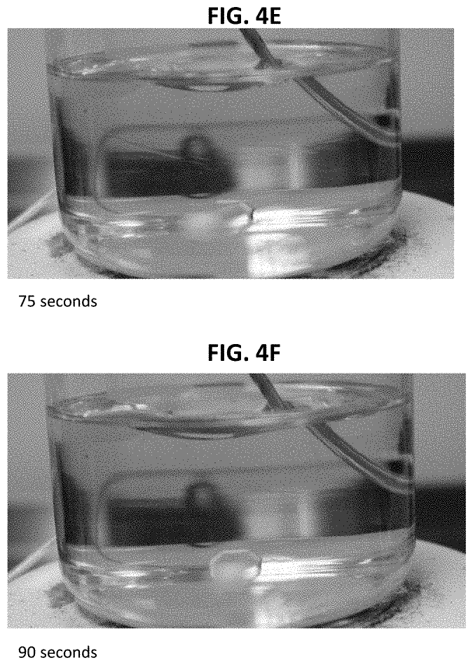

Storage and observation. Both samples were left exposed to the air and monitored for a period of 60 days. For the first 21 days, the samples were mixed daily, videotaped, and photographed in order to observe changes to consistency over time. During observation, both samples were submitted to comparable agitation and mixing. Daily comparative analysis was completed when Sample 1, hemp oil, achieved complete crystallization (day 21) while Sample 2, the therapeutic oil blend, remained in the desired liquid state. On day 60, additional photographs were taken and observations were made. Throughout the experiment, the ambient temperature was maintained at 73° F./23° C. with a relative humidity of 50%±2% RH. Both samples were stored under the same conditions, side by side. Select images of the samples and specific observations of their physical states are provided herein for different time points, as documented in FIGS. 3-4 and the results below. In particular, images and observations are provided for days 1, 7, 14, 15, 16, 21, and 60 for Sample 1, hemp oil; and days 1, 7, 14, 21, and 60 for Sample 2, the therapeutic oil blend.

### Results for Sample 1, Hemp Oil Control

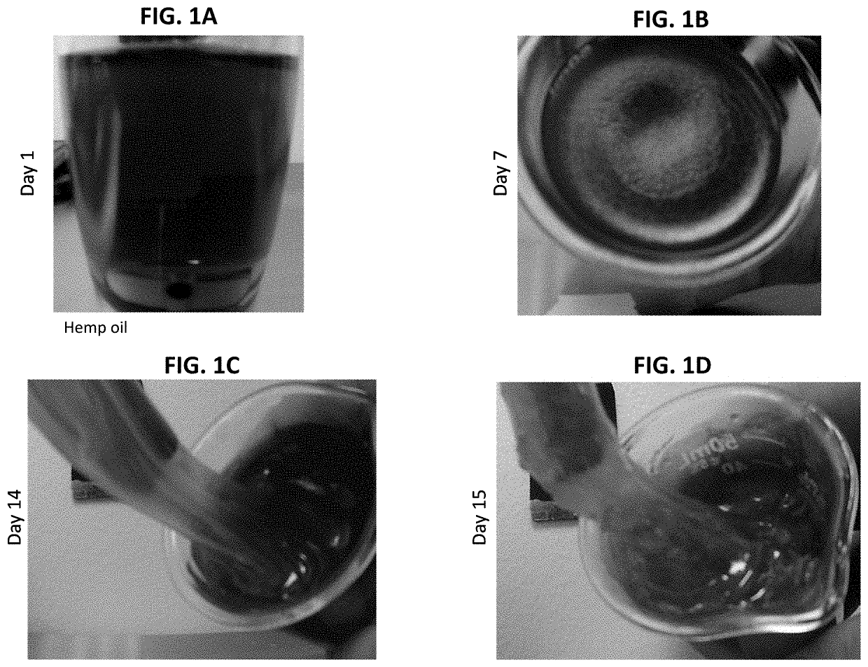

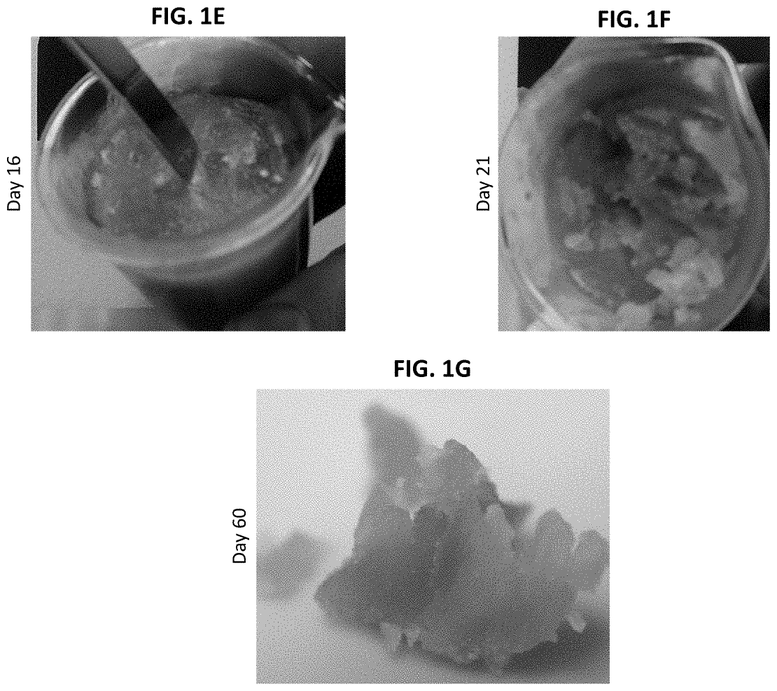

Day 1. Images and observations of Sample 1, hemp oil, were made while the sample was in the process of returning to room temperature. At the point captured in the image (FIG. 1A), the sample was still warm and clear, and brown in color, but the appearance became cloudy as the oil sample cooled further.

Day 7. On day 7, Sample 1 had thickened and could no longer pour. The oil was no longer clear and had formed a very thick, semi-solid material similar in consistency to a resin or sap. See FIG. 1B. This sap-like consistency is typical for freshly produced hemp oil, e.g., that has not been melted at 60° C. On a production scale, the crystallization process can occur rapidly as the hemp oil is placed into multiple containers from one large lot.

Day 14. Sample 1 developed into a very thick resin-like material at 14 days. When stirred with a metal stir rod, the product stuck to the metal rod and stuck to itself. A significant amount of the material stuck to the rod and formed a large strand, similar in consistency to a taffy or pulled caramel. See FIG. 1C.

Day 15. By day 15, chunks of crystals had begun to form in Sample 1. These crystals were clearly apparent in the “chunkiness” of the sample (FIG. 1D). Once the crystallization process starts in hemp oil, the formation of crystals is rapid. These crystals quickly spread throughout the entire mass, which turns solid.

Day 16. At this point, Sample 1 was completely solid (FIG. 1E). This transition to a solid state occurred within 24-48 hours of the start of nucleation. The surface of the mass was hard and required significant pressure to break through. Once the initial stress from the metal implement had broken the surface, the sample exhibited a very hard brown sugar consistency.

Day 21. At day 21, Sample 1 was solid and broke into crystallized chunks when disturbed (FIG. 1F). No further crystallization of the product could occur after this point, since the sample already exhibited full crystallization. Following this time point, the sample only continued to harden and dry further. A fully crystallized hemp oil such as Sample 1 cannot be removed from its container without the application of heat. Daily comparisons ceased at this point, due to the full crystallization of Sample 1.

Day 60. Sample 1 was completely dry and crystalline (FIG. 1G).

### Results for Sample 2, the Therapeutic Oil Blend

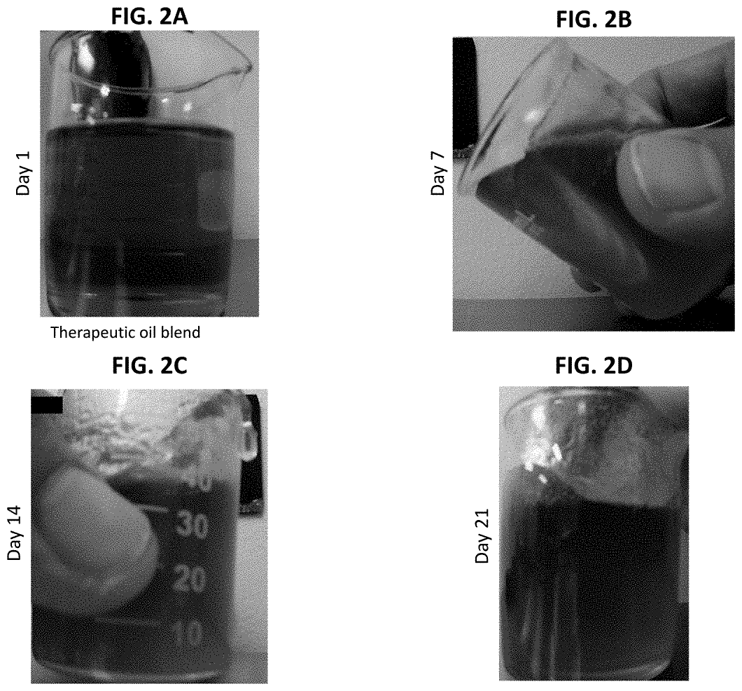

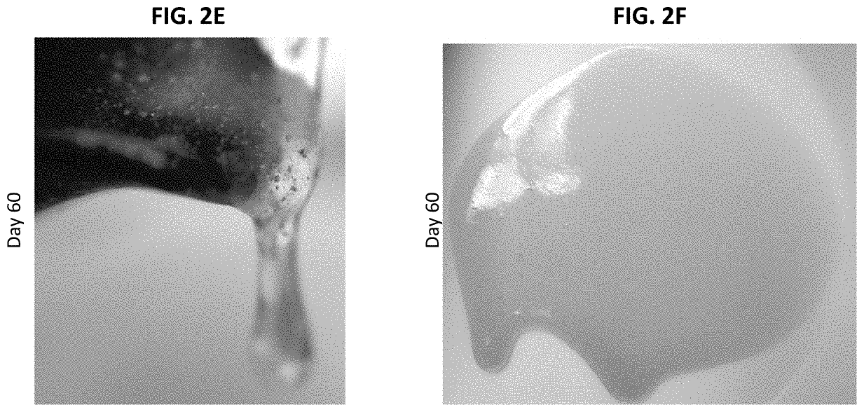

Day 1. An image of Sample 2, the therapeutic oil blend, was similarly taken during its return to room temperature, such that the sample was still warm and clear, and brown in color (FIG. 2A). The product was similar in appearance to Sample 1, however some key differences remained. The refractive index of this mixture was lower than Sample 1, because the exemplary formulation process resulted in a less dense and less viscous mixture, such that light was not bent as sharply when passing through it.

Day 7. On day 7, as shown in FIG. 2B, Sample 2 was still a liquid and could be poured and swirled around the beaker. It also left a thin film on the surfaces it touched. Sample 2 was slightly clouded as the fats and waxes cooled following formulation, just like in the traditional hemp oil Sample 1. However, in contrast to Sample 1, Sample 2 retained its liquid properties. No crystallization was observed.

Day 14. On day 14, Sample 2 was still completely liquified and could still drip down the sides of the beaker, showing its thin viscosity in comparison to standard hemp oil. See FIG. 2C showing a drop of Sample 2 dripping down the side of the beaker. Sample 2 retained its properties and exhibited a runny sap-like consistency. Sample 2 was still crystal-free.

Day 21. By day 21, Sample 2 was still fully in a liquid state, with a consistency slightly thinner than that of maple syrup. In the image contained in FIG. 2D, Sample 2 was being stirred, and the leading edge of the wave is apparent.

Day 60. Even by day 60, Sample 2 was still in a liquid state and retained its consistency. As shown in FIG. 2E, the sample was still liquid and pourable. The sample was poured onto a semi-vertical surface and allowed to drip freely in order to demonstrate its clarity (complete lack of crystallization), its homogeneity (no variation in color or consistency), and its maintained viscosity. See FIG. 2F. While the experiment concluded at this time point, Sample 2 still showed no signs of deterioration in terms of consistency, homogeneity, and shelf stability.

A comparison of Sample 1, the hemp oil control, and Sample 2, the exemplary therapeutic oil blend, at the different time points is provided in Table 5.

TABLE 5
Observations of samples at different time points in open air stability testing.

| | Sample 1, hemp oil, | Sample 2, therapeutic oil |
|---|---|---|
| Day | observations | blend observations |
| 1 | Warm, clear brown liquid | Warm, clear brown liquid following |
| | following melting at | process of Example 3; lower |
| | 60° C. | refractive index |
| 7 | Thick, semi-solid resin; not | Liquid; same homogeneity, |
| | pourable | consistency, and apparent viscosity |
| | | as therapeutic oil blend after |
| | | cooling; no crystallization |
| 14 | Very thick resin; sticks like | Liquid; same homogeneity, |
| | taffy to metal rod | consistency, and apparent viscosity; |
| | | no crystallization |
| 15 | Chunks of crystals forming | Liquid; same homogeneity, |
| | | consistency, and apparent viscosity; |
| | | no crystallization |
| 16 | Completely solid; once | Liquid; same homogeneity, |
| | broken, exhibits hard brown | consistency, and apparent viscosity; |
| | sugar consistency | no crystallization |
| 21 | Fully crystallized | Liquid; same homogeneity, |
| | | consistency, and apparent viscosity; |
| | | no crystallization |
| 60 | Completely dry and | Liquid; same homogeneity, |
| | crystalline | consistency, and apparent viscosity; |
| | | no crystallization |

### Conclusions

Sample 1, a traditional hemp oil, began to crystallize within 14 days after being heated to a completely liquid state on day 1, and was fully solid by day 16 and fully crystallized by day 21. In contrast, over the course of two months of observation in a non-ideal, open air-exposed environment, Sample 2, an exemplary therapeutic oil blend of the disclosure, did not crystallize, change consistency, or exhibit non-homogeneity. Because Sample 2 did not crystallize, less of the product was exposed to air, which lowers the rate of oxidation and maintains the quality and shelf life of this product. In this experiment, the samples were exposed to air and humidity, in contrast to normal storage conditions in which a therapeutic oil blend of the disclosure would be stored in a closed, sealed container away from air and light. Therefore, under normal storage conditions, a therapeutic oil blend of the disclosure such as Sample 2 would be expected to have an even longer shelf life. Crystallization also affects bioavailability of the CBD. Crystalline CBD cannot be absorbed by the skin. Thus, even small amounts of crystallization, such as that occurring by day 15 (or more severely at 16), render the hemp oil less effective for topical applications.

### Example 5: Characterization of Solvation Rate of Exemplary Therapeutic Oil Blend

Traditional hemp oils have a large particle size (and/or crystal size) that slows or inhibits their ability to dissolve in, e.g., carrier oils. Exemplary therapeutic oil blends of the disclosure comprise a smaller particle size that allows for faster solvation and, therefore, greater ease of incorporation into other compositions, such as any of those disclosed herein. To characterize the solvation potential of an exemplary therapeutic oil blend of the disclosure, an assay was performed to compare the speed at which a therapeutic oil blend and two control hemp oils dissolved in a carrier MCT oil. These results are also relevant to absorption speed, as faster solvation correlates with smaller particle size and faster absorption, e.g., via topical application.

### Samples

Sample 1: 10 g of solid traditional hemp oil. (Solid Control)

Sample 2: 10 g of liquid traditional hemp oil. (Liquid Control)

Sample 3: 10 g of an exemplary therapeutic oil blend prepared according to the process disclosed in Example 3 above. (Therapeutic Oil of Present Disclosure)

### Solvation Method

For each sample, 200 g of Medium Chain Triglyceride (MCT) Oil was heated to 60° C. on a hotplate and stirred with a magnetic stir bar running at 200 rpm. Each sample was separately added to their own batch of heated MCT oil all at once and videotaped to determine how long it took to dissolve into the MCT oil. Throughout the experiment, the ambient temperature was maintained at 73° F./23° C. with a relative humidity of 50%±2% RH.

### Results

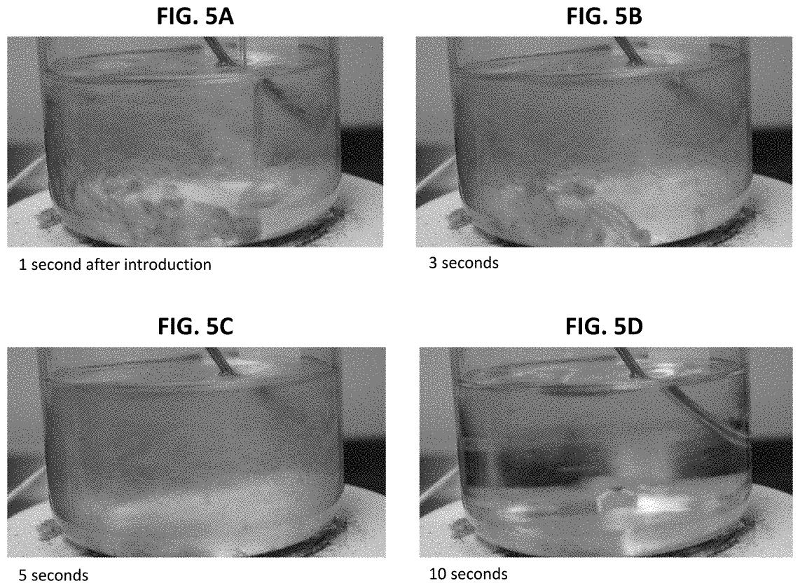

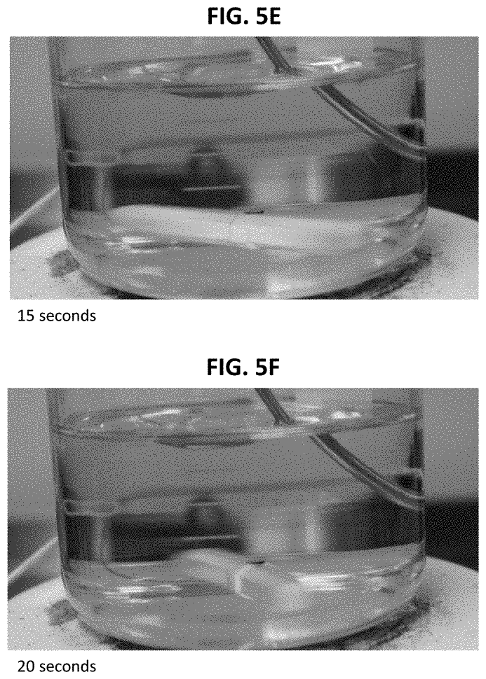

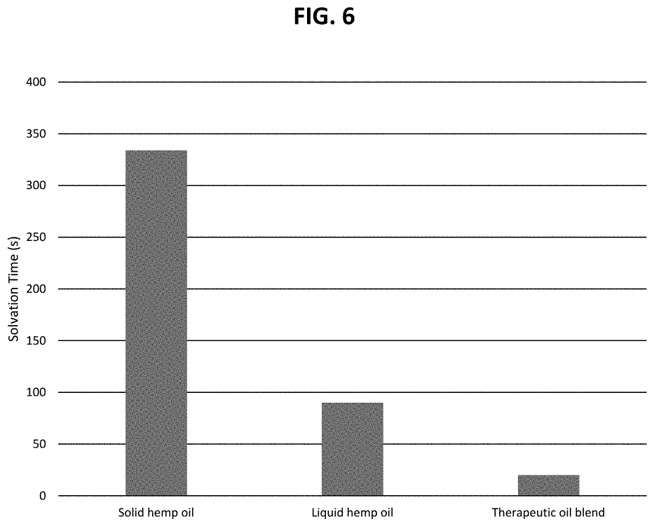

Control Sample 1, solid hemp oil, took 334 seconds to fully dissolve in the MCT oil (FIG. 3A-G). Control Sample 2, liquid hemp oil, took 90 seconds to fully dissolve (FIG. 4A-F). Sample 3, an exemplary therapeutic oil blend of the disclosure, took 20 seconds to fully dissolve (FIG. 5A-F). A bar graph comparing these solvation times is shown in FIG. 6.

These results demonstrate that an exemplary therapeutic oil blend of the disclosure outperformed traditional solid or liquid hemp oil in terms of rate of solvation, as expected given the smaller particle size and nano-penetrative properties of such therapeutic oil blends. This improved solvation property of the therapeutic oil blend allows for improved ease of use in the manufacture of compositions comprising the therapeutic oil blend, as well as resulting in a more homogeneous product on a faster time scale.

### Example 6: In Vivo Absorption Assay

Topical administration of CBD products has become increasingly popular to target the tissues of a localized area. The transdermal absorption of these products is related to the size of the particle of the formulation. Exemplary therapeutic oil blends of the disclosure have a smaller particle size and improved nano-penetrative properties as compared to traditional hemp oils. To test the absorption properties of one such therapeutic oil blend, formulated as in Example 3 above, an in vivo topical absorption assay was performed. The absorption properties of six different topical formulations were evaluated qualitatively using visual analog scales (VAS).

### Samples

Six different samples were prepared:

Sample 1, hemp oil: Pure Hemp oil, comprising approximately 83% CBD. This sample was included to act as a hemp oil only control, without the additional ingredients or process steps involved in formulating the therapeutic oil blend of Example 3.

Sample 2, sonicated hemp oil: Sonicated pure hemp oil, as in Sample 1, comprising approximately 83% CBD. This sample was included to act as a sonication-only hemp oil comparison, experiencing a similar sonication process as the exemplary therapeutic oil blend, but without the added terpenes.

Sample 3, CBD in MCT: Powdered CBD isolate in a carrier MCT Oil, comprising approximately 50% CBD. The organic CBD isolate was obtained from the same pure hemp oil as in Samples 1 and 2. The MCT oil was heated and the CBD isolate was added and stir bar mixed until dissolved and homogeneous. This sample had roughly the same CBD composition as the therapeutic oil blend, but without additional hemp and terpene ingredients.

Sample 4, stirred hemp+terpenes: Pure hemp oil, as in Samples 1 and 2, with added terpenes, comprising approximately 50% CBD. This sample had the same composition as an exemplary therapeutic oil blend prepared according to Example 3 (as in Sample 6), but was only heat and stir bar mixed. This sample included all of the ingredients of the therapeutic oil blend, but without the sonication steps employed to produce it.

Sample 5, CBD+terpenes in MCT: Powdered CBD isolate in a carrier MCT Oil with added terpenes, comprising approximately 50% CBD. The organic CBD isolate was obtained from the same pure hemp oil as in Samples 1 and 2. This sample had the same CBD content and added terpene content as an exemplary therapeutic oil blend prepared according to Example 3 (as in Sample 6), but was formulated by stir bar mixing the ingredients into heated MCT oil. This sample was intended to include all of the “active” ingredients of the therapeutic oil blend in a lighter weight carrier oil.

Sample 6, therapeutic oil blend: An exemplary therapeutic oil blend of the disclosure prepared according to the process disclosed in Example 3.

A comparison of the six samples employed in this experiment is shown in Table 6 below.

TABLE 6
Samples tested in in vivo absorption assay.

| Sample | Ingredients | Formulation process |
|---|---|---|
| 1, hemp oil | Hemp oil | Melted |
| 2, sonicated hemp oil | Hemp oil | Sonicated |
| 3, CBD in MCT oil | Powdered CBD isolate, | Ingredients heated, |
| | MCT oil | stir bar mixed |
| 4, stirred hemp + terpenes | Hemp oil, clove bud oil, | Ingredients heated, |
| | peppermint oil | stir bar mixed |
| 5, CBD + terpenes in MCT | Powdered CBD isolate, | Ingredients heated, |
| | MCT oil, clove bud oil, | stir bar mixed |
| | peppermint oil | |
| 6, therapeutic oil blend | Hemp oil, clove bud oil, | Process of Example 3 |
| | peppermint oil | |

### Method

0.5 ml of each sample was applied to the ventral forearm and gently spread using a single gloved finger from the opposite hand for 20 seconds. VAS were used to collect data on both the visual (how it looked) and tactile (how it felt) absorption. Water was used to clean the area between samples. The measurements were then converted into % scores for each component and combined into a singular absorption score with arbitrary units.

Testing of each sample was performed within one week of formulation. The samples were formulated, shipped via next day air, stored at room temperature away from heat and light and tested within three days of shipping. In spite of this, Sample 1 (hemp oil) had already formed a solid by the time testing commenced, accounting for its absorption score of 0. Of the remaining samples, Sample 6, the therapeutic oil blend, was the only sample that did not exhibit any form of crystallization, separation or precipitation by the time that testing commenced. Each of these samples was manually agitated to mix/resuspend contents prior to absorption testing.

### Results

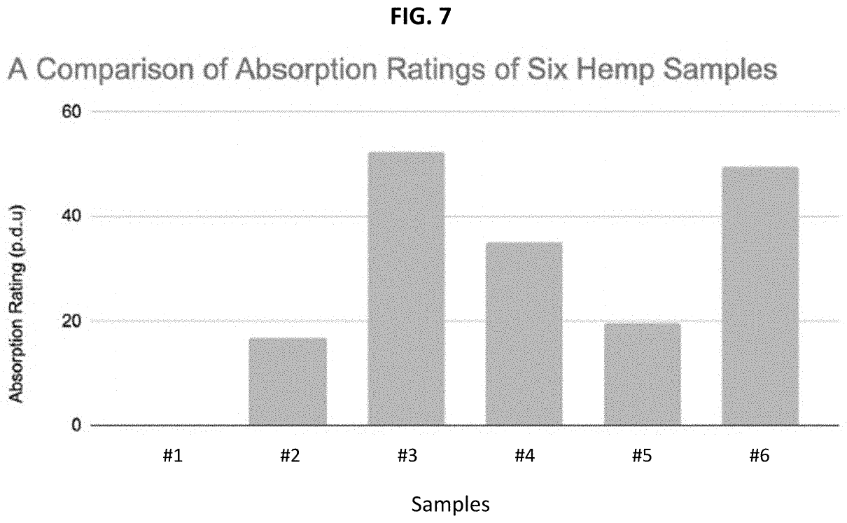

Results of the absorption assay are shown in Table 7 and in FIG. 7, which displays the combined absorption score for each of the six samples. These results show that an exemplary therapeutic oil blend of the disclosure (Sample 6) had significantly improved visual and tactile absorption compared to traditional hemp oil (Sample 1), sonicated hemp oil (Sample 2), and hemp oil with added terpenes (Sample 4). Sample 6, the therapeutic oil blend, compared favorably with an MCT oil composition with added CBD (Sample 3), which is remarkable, given that the MCT oil of Sample 3 had a much lower average carbon chain length than hemp oil, and would therefore have been expected to absorb faster than hemp oils (e.g., see comparison between Sample 2 and 3). In addition, Sample 6 outperformed an MCT oil comprising the same amount of CBD and added terpenes (Sample 5) in terms of absorption. In this sample, the addition of terpenes actually reduced absorption, suggesting that the combination of cannabinoid and terpene contents in the therapeutic oil blend of Sample 6, exhibited unexpected synergistic absorption properties. These results demonstrate that an exemplary therapeutic oil blend of the present disclosure has significantly improved absorption properties compared to existing formulations and even compared to formulations with similar ingredient profiles.

TABLE 7
Absorption ratings for six samples derived from pure hemp oil.

| | | Absorption | Absorption | Absorption | | Combined |
|---|---|---|---|---|---|---|
| | VAS Total | (Visual) - | (Visual) - | (Feel) - | Absorption | Absorption |
| SAMPLE | Measure | Actual | % | Actual | (Feel) - % | Score |
| 1, hemp oil | 163 | 0 | 0 | 0 | 0 | 0 |
| 2, sonicated | 163 | 32 | 20 | 23 | 14 | 17 |
| hemp oil | | | | | | |
| 3, CBD in | 163 | 86 | 53 | 85 | 52 | 52 |
| MCT | | | | | | |
| 4, stirred | 163 | 66 | 40 | 49 | 30 | 35 |
| hemp + | | | | | | |
| terpenes | | | | | | |
| 5, CBD + | 163 | 36 | 22 | 28 | 17 | 20 |
| terpenes in | | | | | | |
| MCT | | | | | | |
| 6, therapeutic | 163 | 85 | 52 | 76 | 47 | 49 |
| oil blend | | | | | | |

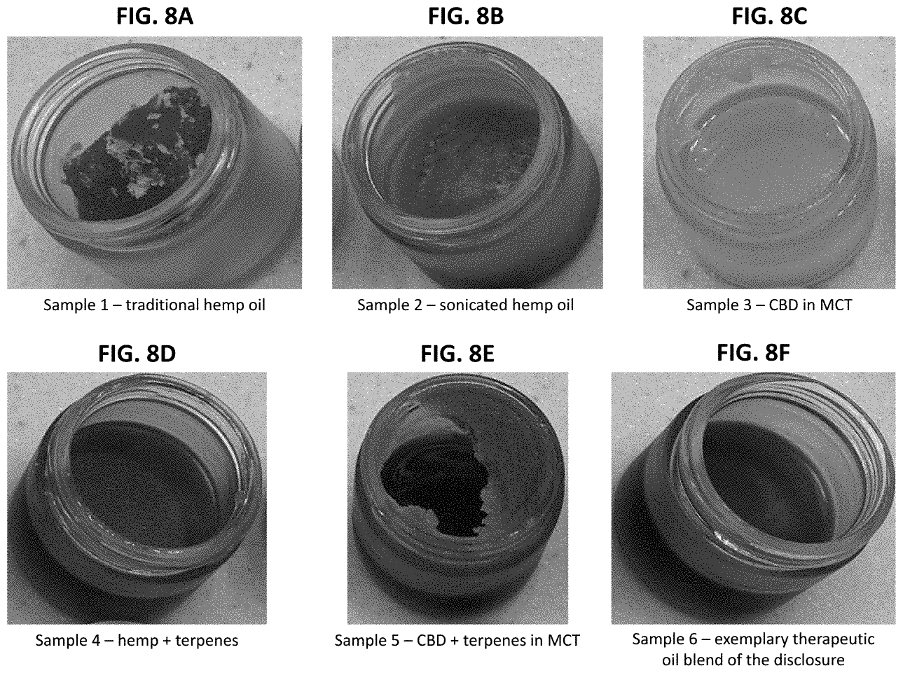

FIG. 8A-F also include photos visually documenting the physical deterioration of the samples at the time of absorption testing, except for Sample 6, the therapeutic oil blend, which was still stable, homogeneous, liquid, and non-crystallized. These observations are tabulated in Table 8, as well. These results further demonstrate that an exemplary therapeutic oil blend of the disclosure exhibits surprisingly improved shelf life and stability, in addition to its improved absorption properties, compared to traditional hemp oils and CBD terpene blends.

TABLE 8
Observations on physical state of sample at time of absorption testing.

| SAMPLE | Observations |
|---|---|
| 1, hemp oil | Sample was solid with visible cracks on surface |
| 2, sonicated hemp oil | Exhibited significant crystallization |
| 3, CBD in MCT | Some precipitation observed |
| 4, stirred hemp + | Gritty texture from precipitation and/or |
| terpenes | crystallization of constituents |
| 5, CBD + terpenes in | Significant precipitation |
| MCT | |
| 6, therapeutic oil | No crystallization, precipitation, or |
| blend | non-homogeneity |

### Example 7: In Vivo Thermography Assay for Topical Application

Traditional hemp oil and CBD products do not exhibit pronounced vasodilatory effects, e.g., when applied topically to the skin. In contrast, exemplary therapeutic oil blends of the disclosure, such as those formulated via the processes disclosed in Examples 1-3 are expected to induce vasodilation as a result of their terpene composition. This vasodilation is important for the rapid delivery of active ingredients to the target area and for penetration of sensory nerve fibers. To characterize the vasodilatory effect of an exemplary therapeutic oil blend, a thermography assay was performed. Thermography is an indirect method of measuring a vasodilation response via a change in skin temperature which occurs with increased blood flow.

### Samples

Two samples were prepared:

Sample 1, CBD in MCT: powdered CBD isolate dissolved in MCT Oil, comprising approximately 50% CBD. For this sample, a powdered organic CBD isolate was stir bar mixed into heated MCT oil. This sample represents a CBD only comparison, without the other “active” ingredients comprised by the therapeutic oil blend.

Sample 2, therapeutic oil blend: A therapeutic oil blend was prepared according to the process disclosed in Example 3.

### Method

0.5 ml of each sample was applied to the ventral forearm and gently spread using a single gloved finger from the opposite hand for 20 seconds. Thermal images were taken using a FLIR C2 (Flir Systems, Sweden) immediately prior to application, 30 seconds after application, and 60 seconds after application.

### Results

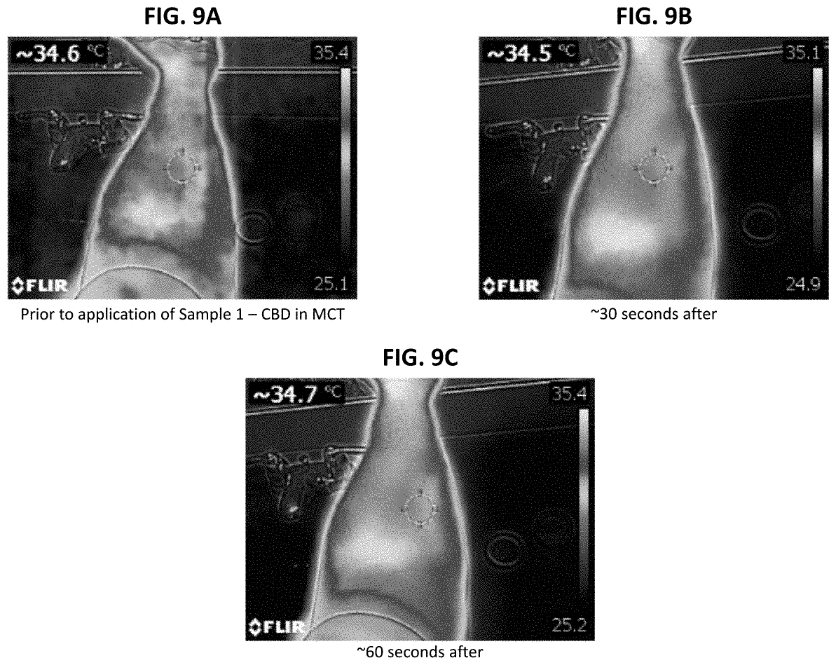

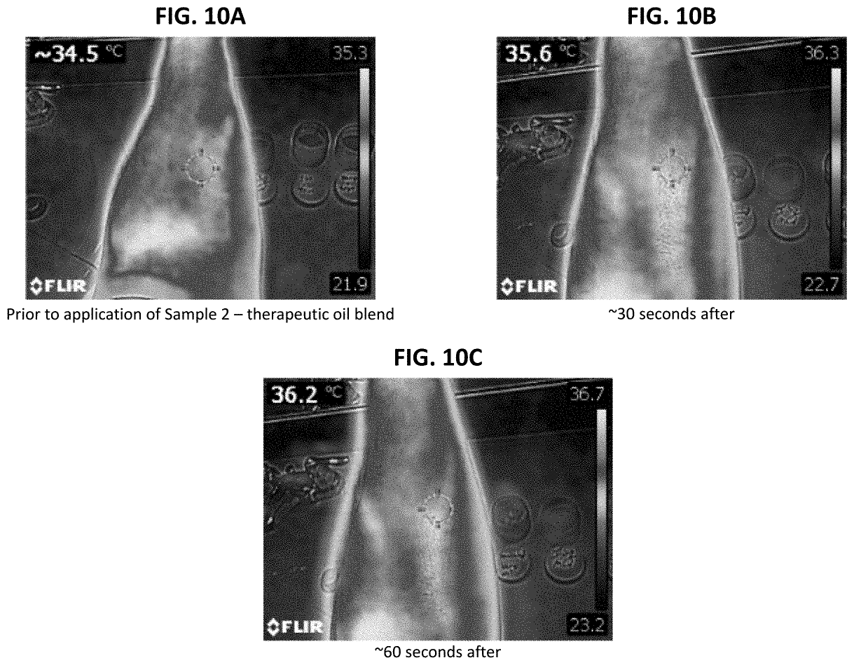

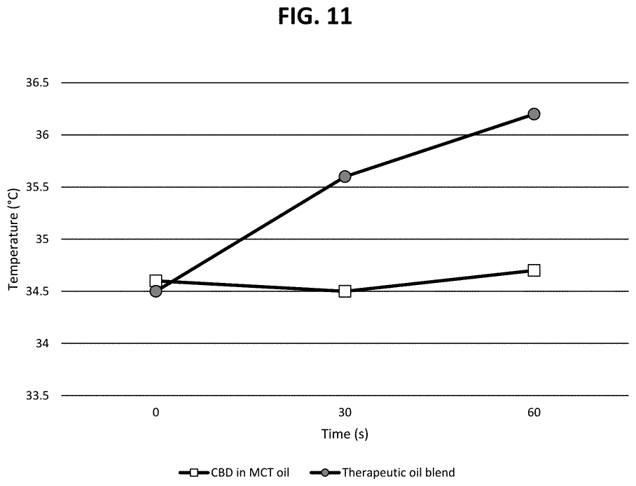

Results of this thermography assay are shown in FIG. 9A-C and FIG. 10A-C. The images in FIG. 9A-C demonstrate essentially no variation in skin temperature induced by application of Sample 1 at the time points measured. By contrast, application of the therapeutic oil blend resulted in an increase in skin temperature of ˜1.1° C. at 30 s post application and an increase of ˜1.7° C. at 60 s post application (FIG. 10A-C). A line graph comparing the temperature changes for each sample is shown in FIG. 11, and these results are also tabulated in Table 9 below. These results illustrate the vasodilatory characteristics of an exemplary therapeutic oil blend of the disclosure in contrast to a CBD only control.

TABLE 9
Results of thermographic assay.

| | Temperature (° C.) | |
|---|---|---|
| | Sample 1, CBD | Sample 2, therapeutic |
| Time point | in MCT | oil blend |
| Prior to application | 34.6 | 34.5 |
| 30 s post-application | 34.5 | 35.6 |
| 60 s post-application | 34.7 | 36.2 |

### Example 8: In Vivo Assay Demonstrating Anesthetic Properties of Exemplary Therapeutic Oil Blend

Traditional hemp oils and CBD oils do not have anesthetic properties, in contrast to exemplary therapeutic oil blends of the disclosure which do have this property. To demonstrate this, an exemplary therapeutic oil blend of the disclosure was formulated according to Example 2 above. The therapeutic oil blend was tested via a standard skin prick assay to determine its ability to reduce tactile sensation, e.g., pain stimuli, when applied topically.

### Method

Three human subjects participated in this study. Each subject was scratched with a tip of a paperclip at neutral pressure on their mid forearm and were told to rate that stimulus as a 5 as the level of baseline stimulus.

Five minutes later, each subject took 500 mg of the therapeutic oil blend and rubbed it into the spot on their mid forearm for 45 seconds. The subjects then waited approximately 1 minute before re-administering the scratch test and then rated the scratch compared to the baseline result on a scale of 1-5, with 1 being little sensation and 5 being identical to the baseline sensation.

Ten minutes later, subjects re-administered the scratch test and again rated the sensation on a scale of 1-5.

### Results

The results of the assay are shown in Table 10 below for the scratch test performed one minute after application of the therapeutic oil blend (“one minute test”) and for the scratch test performed ten minutes after that (“ten minute test”), as compared to the baseline scratch test.

TABLE 10
Scratch test results.

| | Baseline | One minute test | Ten minute test |
|---|---|---|---|
| Subject A | 5 | 2 | 3 |
| Subject B | 5 | 3 | 3 |
| Subject C | 5 | 1 | 2 |

These results demonstrate an average reduction of 60% in tactile sensation within one minute of application of an exemplary therapeutic oil blend of the disclosure, and an average reduction of 47% in tactile sensation ten minutes following application. These results also illustrate that the smaller particle size and nano-penetrative properties of the therapeutic oil blend allow the product to penetrate to the sensory nerve fibers, as indicated by the fact that the application of the product reduces tactile sensation on the areas applied (numbing). These tests were informally repeated in the course of performing the assays of Examples 7 and 8 below, in which context they were compared to hemp oil, sonicated hemp oil, and CBD isolate in MCT oil. These comparisons confirmed that the exemplary therapeutic oil blend of the present invention had anesthetic properties not possessed by traditional hemp and CBD oils.

### Example 9: Microscopy-Based Analysis of Particle Size

A sample of hemp oil and an exemplary therapeutic oil blend prepared according to Example 3 above, and derived from the same type of hemp oil, were visually compared under a microscope at 4×, 10×, and 20× magnification to compare the particle sizes comprised by each sample. In particular, the samples were analyzed to visually identify the size of CBD crystal particulates comprised by each.

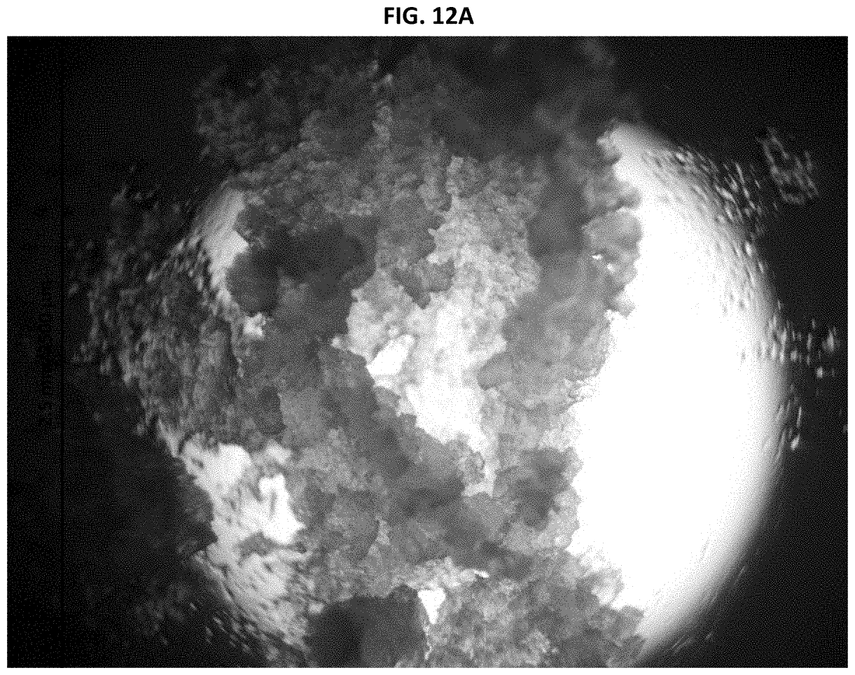

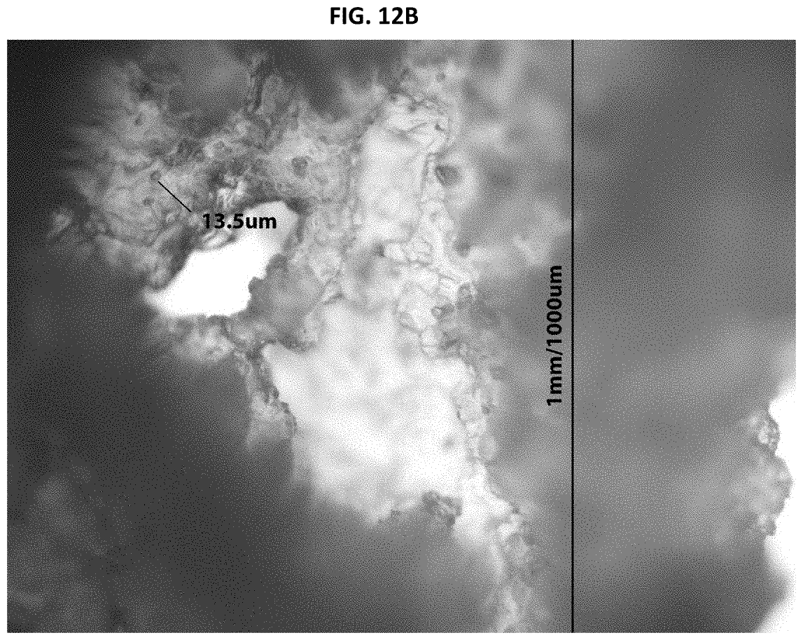

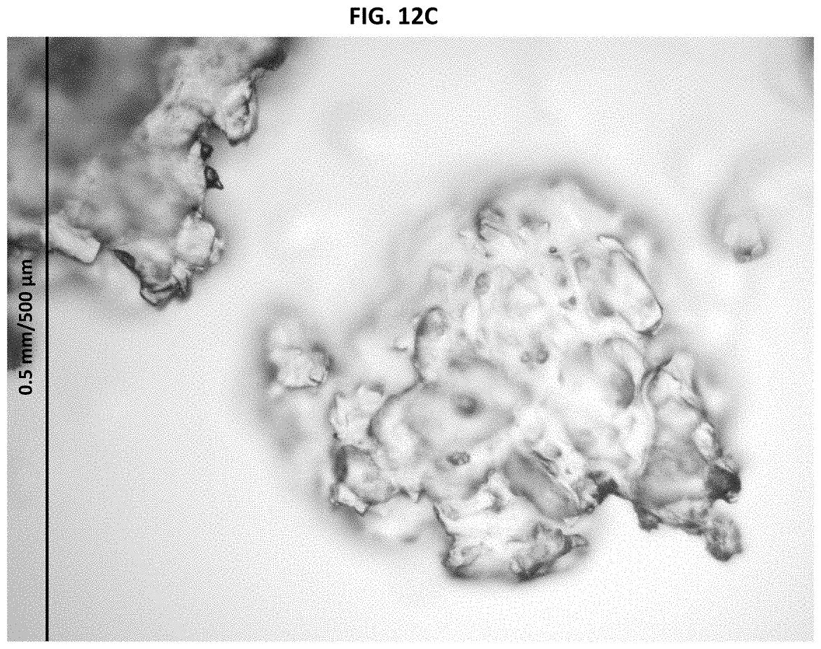

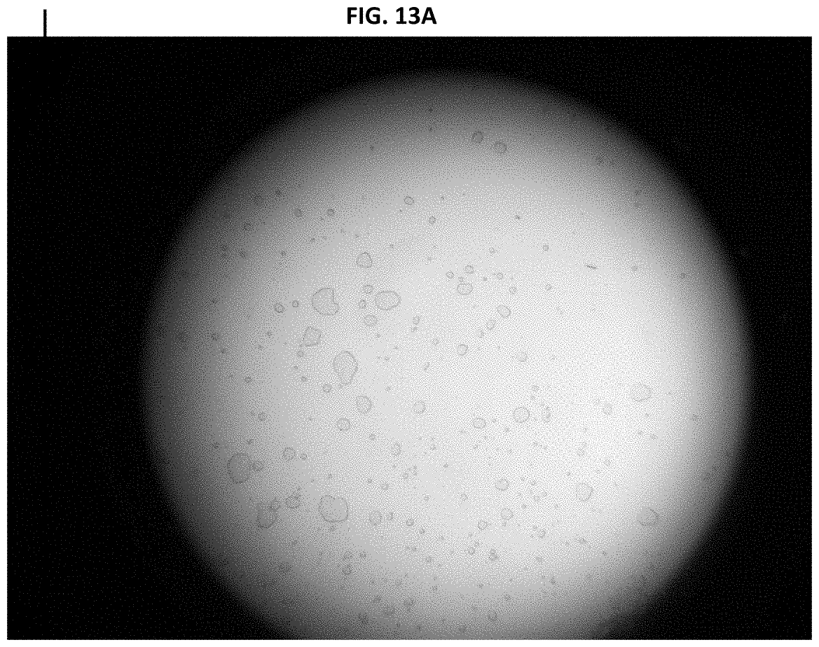

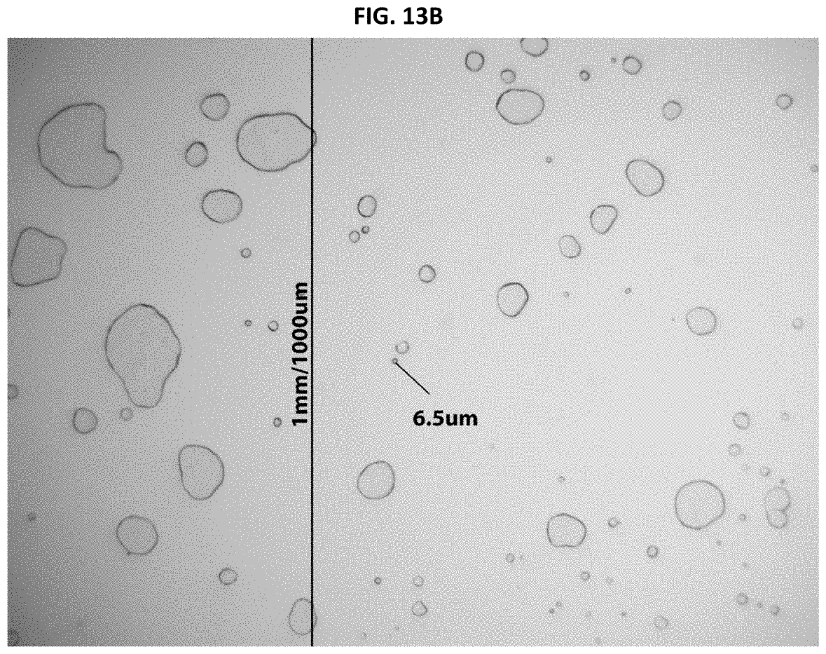

As shown in FIG. 12A-C, the hemp oil contained large crystalline structures, measuring multiple millimeters across in length (FIG. 12A). FIG. 12C shows a smaller crystalline structure that is nevertheless about 0.3 mm in diameter. In contrast, while FIG. 13A-C show air bubbles dispersed throughout the sample, there are no detectable crystals or particulates of any kind at these magnifications within the therapeutic oil blend. These images demonstrate that the particle size of the therapeutic oil blend is well below 0.1 μM, as a particle of this size would be visible, e.g., at 20× magnification.

### Example 10: Freeze-Thaw Stability Testing

Freeze-thaw stability testing is commonly used, e.g., in the cosmetics industry, to determine the temperature stability of a product, which is especially relevant for transportation of the product without undesirable changes, such as separation or consistency changes.

### Samples

A sample of full spectrum CBD-enriched hemp oil (Sample 1) and a sample of an exemplary therapeutic oil blend of the disclosure (Sample 2) were tested via iterated cycles of freezing and thawing as a measure of physical stability and in order to determine changes in organoleptic profile as a result of temperature abuse. Sample 2 was formulated using the same full spectrum CBD-enriched hemp oil comprised by Sample 1 and was prepared according to the process disclosed in Example 3 above. Additional control samples for each composition were maintained at 20° C. throughout the experiment to serve as a baseline for non-temperature abused material.

### Freeze-Thaw Conditions

The samples were alternately exposed to a cycle of 48 hours in freezing conditions (0° C. in a freezer) and 48 hours in thawing conditions (25° C. in an incubator, 15% RH) for a total of three cycles.

### Results

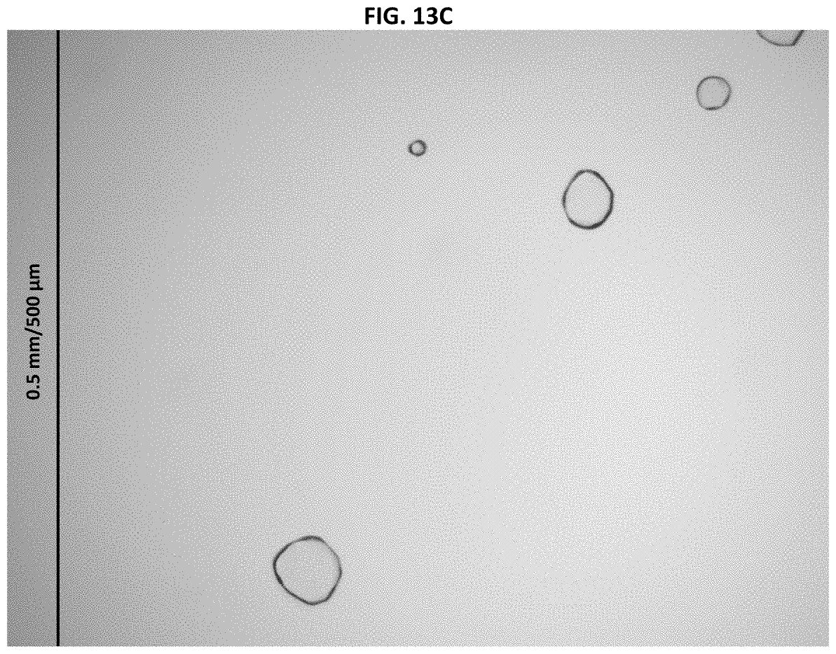

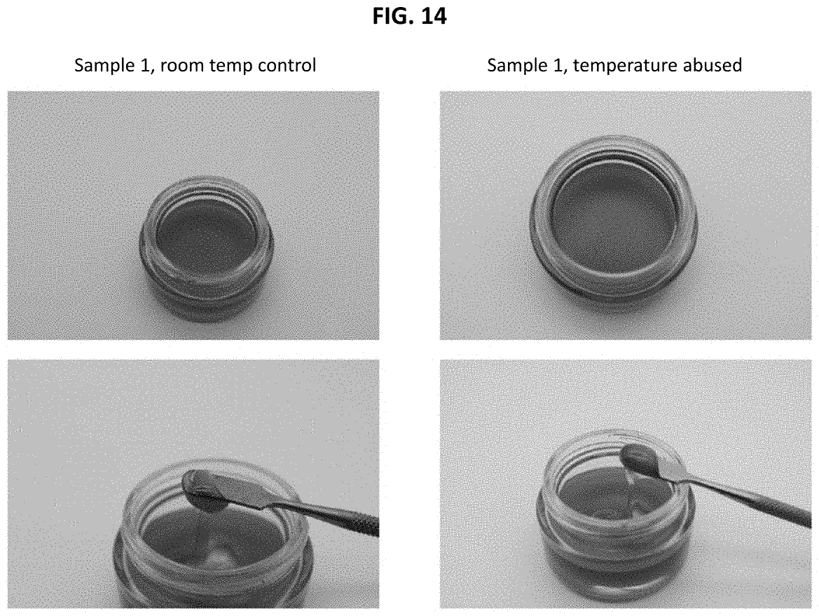

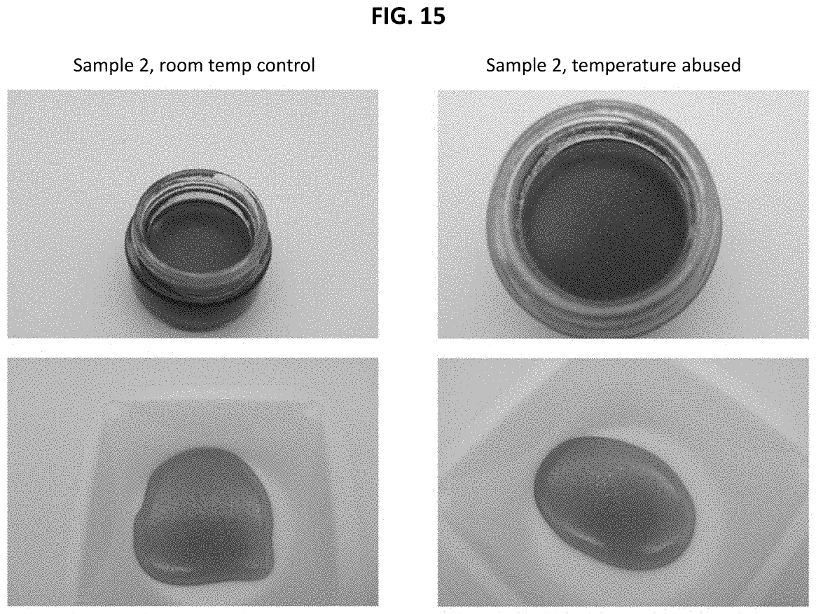

At the end of all three cycles of freezing and thawing, Sample 1 (hemp oil) experienced some changes in organoleptic profile: it was slightly less sticky and more easily spreadable than the control and exhibited a slight change in smell, with an increase in plant/hemp odor, though no crystallization was observed. FIG. 14 shows a comparison between the room temperature control and temperature-abused Sample 1. In contrast, Sample 2, an exemplary therapeutic oil blend of the disclosure was identical to its respective room temperature control, with an equivalent flow rate and no observable changes to the material. These results demonstrate that therapeutic oil blends of the disclosure have stable organoleptic profiles throughout freeze-thaw testing, and would be expected to maintain their physical attributes when exposed to typical transportation-related fluctuations in temperature. FIG. 15 shows a comparison between the room temperature control and temperature-abused Sample 2.

### Example 11: Topical Administration of Therapeutic Oil Blend

A nano-penetrative therapeutic oil blend comprising a cannabidiol-related cannabinoid, eugenol, menthol, menthone, and d-limonene formulated as in examples 1-4 is administered topically to subjects in a fitness environment following a period of physical exertion. Subjects experience decreased pain and muscle tension and improved recovery time. The smaller particle size and stable, homogeneous mixture, is capable of penetrating to the sensory nerve fibers, as indicated by the fact that the application of the product reduces tactile sensation on the areas applied (numbing). A control blend formulated through simple mixing of the ingredients is not able to penetrate the dermis to the same degree and/or in the same rapid time period, as indicated by an absence of numbing, reduced numbing, and/or delay of numbing.

### Example 12: Formulation of Lotion Comprising Therapeutic Oil Blend and Arnica Oil

A lotion is formulated by mixing an emulsifier with a therapeutic oil blend according to the present disclosure, carrier oils, and essential oils, including Arnica oil, to produce an emulsion with water. An emulsifier such as cetyl alcohol or stearic acid is added to the oil phase. The water phase is prepared separately and may contain stabilizing agents. The mixture is heated to between 45 and 85° C. and mixed until a stable emulsion is formed. The lotion comprises 150 mg of cannabidiol or a cannabidiol-related cannabinoid per ounce of lotion.

### Example 13: Formulation of Lotion Comprising Therapeutic Oil Blend and Rose Absolute Oil

A lotion is prepared as in Example 12, but comprising Rose absolute oil and other essential oils instead of Arnica oil. The lotion comprises 150 mg of cannabidiol or a cannabidiol-related cannabinoid per ounce of lotion.

### Example 14: Formulation of Body Balm Comprising Therapeutic Oil Blend and Turmeric CO₂ Oil

A body balm is prepared by mixing shea butter, beeswax, a therapeutic oil blend, turmeric CO₂ oil and other essential oils and carrier oils. The mixture is heated until the ingredients are well-blended and then cooled to solid form. The body balm comprises 500 mg of cannabidiol or a cannabidiol-related cannabinoid per ounce of body balm.

### Example 15: Formulation of Sleep Tincture Comprising Therapeutic Oil Blend and Valerian Root Extract

A sleep tincture is prepared by mixing MCT oil, a therapeutic oil blend, valerian root extract, and essential oils. The sleep tincture comprises 1000 mg of cannabidiol or a cannabidiol-related cannabinoid per ounce of sleep tincture.

### Example 16: Illustrative Compositions Comprising an Exemplary Therapeutic Oil Blend of the Disclosure

The therapeutic oil blends of the present invention are incorporated into the following compositions.

This composition comprises the following ingredients: purified water, glyceryl stearate, cetearyl alcohol, sodium stearoyl lactylate, organic olive oil, organic sunflower oil, organic glycerin, organic hempseed oil, Lactobacillus ferment, Lactobacillus, Cocos nucifera (coconut) fruit extract, organic menthol, white camphor oil, organic eucalyptus oil, organic black pepper oil, organic arnica oil, therapeutic oil blend (as in Example 4), hyaluronic acid, organic turmeric CO₂ oil, mixed tocopherols (vitamin E), and organic aloe vera powder. This composition comprises 300 mg CBD in each 2 ounces.

This composition comprises the following ingredients: purified water, glyceryl stearate, cetearyl alcohol, sodium stearoyl lactylate, organic olive oil, organic sunflower oil, organic glycerin, organic hempseed oil, Lactobacillus ferment, Lactobacillus, Cocos nucifera (coconut) fruit extract, organic Indian peppermint oil, organic arnica oil, lemon balm oil, organic eucalyptus oil, organic rose absolute oil, therapeutic oil blend (as in Example 4), organic chamomile oil, hyaluronic acid, aloe vera powder, and mixed tocopherols (vitamin E). This composition comprises 300 mg CBD in each 2 ounces.

This composition comprises the following ingredients: organic refined shea butter, organic beeswax, organic MCT coconut oil, organic turmeric CO₂ oil, therapeutic oil blend (as in Example 4), organic arnica oil, organic menthol, white camphor oil, and organic eucalyptus oil. This composition comprises 500 mg CBD in each 1 ounce or 835 mg CBD in each 1.67 ounces.

This composition comprises the following ingredients: organic MCT, therapeutic oil blend (as in Example 4), organic valerian root extract, organic lemon balm oil, organic chamomile oil, organic myrcene, and organic lavender oil. This composition comprises 1,000 mg (1 g) CBD in each 1 ounce.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world.

### NUMBERED EMBODIMENTS OF THE INVENTION

Notwithstanding the claims provided herein, the present disclosure contemplates the following embodiments:

1. A mechanically agitated, non-crystallizing liquid therapeutic oil blend with emulsion-like properties comprising:

a) 30-90% w/w cannabinoid oil; and

b) 25-75% w/w of one or more essential oils.

2. A cavitated, non-crystallizing liquid therapeutic oil blend with emulsion-like properties comprising:

a) 30-90% w/w cannabinoid oil; and

b) 25-75% w/w of one or more essential oils.

3. A sonicated, non-crystallizing liquid therapeutic oil blend with emulsion-like properties comprising:

a) 30-90% w/w cannabinoid oil; and

b) 25-75% w/w of one or more essential oils.

4. A liquid therapeutic oil blend that does not crystallize at room temperature for a period of at least 60 days comprising:

a) 30-90% w/w cannabinoid oil; and

b) 25-75% w/w of one or more essential oils.

5. A liquid therapeutic oil blend with emulsion-like properties that has an average particle size of less than 0.1 μM comprising:

a) 30-90% w/w cannabinoid oil; and

b) 25-75% w/w of one or more essential oils.

6. A mechanically agitated, non-crystallizing liquid therapeutic oil blend with emulsion-like properties comprising:

a) 25-75% w/w cannabinoid, and

b) 25-75% w/w terpenes.

7. A cavitated, non-crystallizing liquid therapeutic oil blend with emulsion-like properties comprising:

a) 25-75% w/w cannabinoid, and

b) 25-75% w/w terpenes.

8. A sonicated, non-crystallizing liquid therapeutic oil blend with emulsion-like properties comprising:

a) 25-75% w/w cannabinoid, and

b) 25-75% w/w terpenes.

9. A liquid therapeutic oil blend that does not crystallize at room temperature for a period of at least 60 days comprising:

a) 25-75% w/w cannabinoid, and

b) 25-75% w/w terpenes.

10. A liquid therapeutic oil blend with emulsion-like properties that has an average particle size of less than 0.1 μM comprising:

a) 25-75% w/w cannabinoid, and

b) 25-75% w/w terpenes.

11. A mechanically agitated, non-crystallizing liquid therapeutic oil blend with emulsion-like properties comprising:

a) 30-90% w/w cannabinoid oil;

b) 15-40% w/w clove bud oil; and

c) 5-20% w/w peppermint oil.

12. A cavitated, non-crystallizing liquid therapeutic oil blend with emulsion-like properties comprising:

a) 30-90% w/w cannabinoid oil;

b) 15-40% w/w clove bud oil; and

c) 5-20% w/w peppermint oil.

13. A sonicated, non-crystallizing liquid therapeutic oil blend with emulsion-like properties comprising:

a) 30-90% w/w cannabinoid oil;

b) 15-40% w/w clove bud oil; and

c) 5-20% w/w peppermint oil.

14. A liquid therapeutic oil blend that does not crystallize at room temperature for a period of at least 60 days comprising:

a) 30-90% w/w cannabinoid oil;

b) 15-40% w/w clove bud oil; and

c) 5-20% w/w peppermint oil.

15. A liquid therapeutic oil blend with emulsion-like properties that has an average particle size of less than 0.1 μM comprising:

a) 30-90% w/w cannabinoid oil;

b) 15-40% w/w clove bud oil; and

c) 5-20% w/w peppermint oil.

16. The therapeutic oil blend of any one of embodiments 1-15, wherein the therapeutic oil blend has been cavitated.
17. The therapeutic oil blend of any one of embodiments 1-16, wherein the therapeutic oil blend has been sonicated.
18. The therapeutic oil blend of any one of embodiments 1-17, wherein the therapeutic oil blend is homogeneous.
19. The therapeutic oil blend of any one of embodiments 1-18, wherein the therapeutic oil blend is liquid at room temperature.
20. The therapeutic oil blend of any one of embodiments 1-19, wherein the therapeutic oil blend is liquid at room temperature for a period of at least 60 days.
21. The therapeutic oil blend of any one of embodiments 1-20, wherein the therapeutic oil blend is shelf stable.
22. The therapeutic oil blend of any one of embodiments 1-21, wherein the therapeutic oil blend is shelf stable for a period of at least 60 days.
23. The therapeutic oil blend of any one of embodiments 1-22, wherein the therapeutic oil blend comprises 30-90% w/w of the cannabinoid oil.
24. The therapeutic oil blend of any one of embodiments 1-23, wherein the therapeutic oil blend comprises 25-75% w/w cannabinoid.
25. The therapeutic oil blend of any one of embodiments 1-24, wherein the cannabinoid oil comprises cannabidiol (CBD).
26. The therapeutic oil blend of any one of embodiments 1-25, wherein the cannabinoid oil comprises cannabidiolic acid (CBDA).
27. The therapeutic oil blend of any one of embodiments 1-26, wherein the cannabinoid oil comprises cannabidiol monomethylether (CBDM).
28. The therapeutic oil blend of any one of embodiments 1-27, wherein the cannabinoid oil comprises cannabidiol-C4 (CBD-C4).
29. The therapeutic oil blend of any one of embodiments 1-28, wherein the cannabinoid oil comprises cannabidivarinic acid (CBDVA).
30. The therapeutic oil blend of any one of embodiments 1-29, wherein the cannabinoid oil comprises cannabidivarin (CBDV).
31. The therapeutic oil blend of any one of embodiments 1-30, wherein the cannabinoid oil comprises cannabidiorcol (CBD-C1).
32. The therapeutic oil blend of any one of embodiments 1-31, wherein the cannabinoid oil comprises delta 9 tetrayhydrocannabinol (THC).
33. The therapeutic oil blend of any one of embodiments 1-32, wherein the therapeutic oil blend comprises 25-30%, 30-35%, 35-40%, 40-45%, 45-50%, 50-55%, 55-60%, 60-65%, 65-70%, or 70-75% w/w cannabinoids.
34. The therapeutic oil blend of any one of embodiments 1-33, wherein the therapeutic oil blend comprises 25-30%, 30-35%, 35-40%, 40-45%, 45-50%, 50-55%, 55-60%, 60-65%, 65-70%, or 70-75% w/w CBD.
35. The therapeutic oil blend of any one of embodiments 1-34, wherein the cannabinoid oil comprises hemp oil.
36. The therapeutic oil blend of any one of embodiments 1-35, wherein the cannabinoid oil comprises a cannabinoid isolate.
37. The therapeutic oil blend of any one of embodiments 1-36, wherein the cannabidiol oil comprises a cannabis extract.
38. The therapeutic oil blend of any one of embodiments 1-37, wherein the cannabinoid oil comprises at least 30%, 40%, 50%, 60%, 70%, or 80% cannabinoids.
39. The therapeutic oil blend of any one of embodiments 1-38, wherein the cannabinoid oil comprises at least 30%, 40%, 50%, 60%, 70%, or 80% CBD.
40. The therapeutic oil blend of any one of embodiments 1-39, wherein the one or more essential oils are selected from the group consisting of Linalool; B-Caryophyllene; B-Myrcene; D-Limonene; Humulene; a-Pinene; Ylang Ylang; Yarrow; Violet; Vetiver; Vanilla; Tuberose; Thyme; Tea Tree; Tangerine; Spruce, Black; Spruce; Spikenard; Spearmint; Sandalwood; Rosewood; Rosemary Verbenone; Rosemary; Rose; Rose Geranium; Ravensara; Plai Pine Needle; Petitgrain; Peppermint; Pepper, Black; Patchouli; Palo Santo; Palmarosa; Osmanthus; Oregano; Orange, Sweet; Oak Moss; Nutmeg Niaouli; Neroli; Myrtle; Myrrh; Mimosa; Melissa; Marjoram, Sweet; Manuka; Mandarin, Red; Mandarin; Lotus, White; Lotus, Pink; Lotus, Blue; Lime; Lily; Lemongrass; Lemon; Lavender; Lavandin; Kanuka; Juniper Berry; Jasmine; Jasmine Abs; Helichrysum; Grapefruit, White; Grapefruit, Pink; Ginger; Geranium; Geranium, Bourbon; Gardenia; Galbanum; Frankincense; Frangipani; Fir Needle White; Fir Needle Siberia; Fir Needle Canada; Fennel, Sweet; Eucalyptus Smithii. Eucalyptus Radiata, Eucalyptus Globulus, Eucalyptus Citriodora, Eucalyptus Blue Mallee; Elemi; Dill; Cypress; Cumin; Coriander; Cocoa; Clove; Clary Sage; Cistus; Cinnamon; Chamomile, Roman; Chamomile, Blue; Celery Seed; Cedarwood, Western Red; Cedarwood, Blood; Cedarwood Atlas; Carrot Seed; Cardamon; Caraway Seed; Cajeput; Cade; Birch, White; Birch, Sweet; Bergamot; Bay Laurel; Basil; Basil, Holy; Basil; Balsam Poplar; Balsam Peru; Angelica; and combinations thereof.
41. The therapeutic oil blend of any one of embodiments 1-40, wherein the one or more essential oils are obtained from one or more plants or plant parts selected from the group consisting of Alfalfa; Allspice; Almont, bitter; Ambrette; Angelica root; Angelica seed; Angelica stem; Angostura; Anise; Asafetida; Balm; Balsam of Peru; Basil; Bay leaves; Bay; Bergamot; Bitter almond; Bois de rose; Cacao; Camomile flowers, Hungarian; Camomile flowers, Roman or English; Cananga; Capsicum; Caraway; Cardamom seed; Carob bean; Carrot; Cascarilla bark; Cassia bark, Chinese; Cassia bark, Padang or Batavia; Cassia bark, Saigon; Celery seed; Cherry, wild, bark; Chervil Hoffm.); Chicory; Cinnamon bark, Ceylon; Cinnamon bark, Chinese; Cinnamon bark, Saigon; Cinnamon leaf, Ceylon; Cinnamon leaf, Chinese; Cinnamon leaf, Saigon; Citronella; Citrus peels; Clary; Clover; Coca; Coffee; Cola nut; Coriander; Cumin; Curacao orange peel; Cusparia bark; Dandelion; Dandelion root; Dog grass; Elder flowers; Estragole; Estragon; Fennel, sweet; Fenugreek; Galanga; Geranium; Geranium, East Indian; Geranium, rose; Ginger; Grapefruit; Guava; Hickory bark; Horehound; Hops; Horsemint; Hyssop; Immortelle; Jasmine; Juniper; Kola nut; Laurel berries; Laurel leaves; Lavender; Lavender, spike; Lavandin; Lemon; Lemon balm; Lemon grass; Lemon peel; Lime; Linden flowers; Locust bean; Lupulin; Mace; Mandarin; Marjoram, sweet; Mate; Melissa; Menthol; Menthyl acetate; Molasses; Mustard; Naringin; Neroli, bigarade; Nutmeg; Onion; Orange, bitter, flowers; Orange, bitter, peel; Orange leaf; Orange, sweet; Orange, sweet, flowers; Orange, sweet, peel; Origanum; Palmarosa; Paprika; Parsley; Pepper, black; Pepper, white; Peppermint; Peruvian balsam; Petitgrain; Petitgrain lemon; Petitgrain mandarin or tangerine; Pimenta; Pimenta leaf; Pipsissewa leaves; Pomegranate; Prickly ash bark; Rose absolute; Rose; Rose buds; Rose flowers; Rose fruit; Rose geranium; Rose leaves; Rosemary; Saffron; Sage; Sage, Greek; Sage, Spanish; St. John's bread; Savory, summer; Savory, winter; Schinus molle; Sloe berries; Spearmint; Spike lavender; Tamarind; Tangerine; Tarragon; Tea; Thyme; Thyme, white; Thyme, wild or creeping; Triticum; Tuberose; Turmeric; Vanilla; Violet flowers; Violet leaves; Violet leaves absolute; Wild cherry bark; Ylang-ylang; Zedoary bark; and combinations thereof.
42. The therapeutic oil blend of any one of embodiments 1-41, wherein the one or more essential oils comprise clove bud oil and peppermint oil.
43. The therapeutic oil blend of any one of embodiments 1-42, wherein the therapeutic oil blend comprises about 15-40% w/w clove bud oil.
44. The therapeutic oil blend of any one of embodiments 1-43, wherein the therapeutic oil blend comprises about 5-20% w/w peppermint oil.
45. The therapeutic oil blend of any one of embodiments 1-44, wherein the therapeutic oil blend comprises about 15-40% w/w clove bud oil and 5-20% w/w peppermint oil.
46. The therapeutic oil blend of any one of embodiments 1-45, wherein the therapeutic oil blend does not comprise any exogenously added emulsifiers, surfactants, or other homogenizing agents, other than the terpenes.
47. The therapeutic oil blend of any one of embodiments 1-46, wherein the therapeutic oil blend is not an emulsion.
48. The therapeutic oil blend of any one of embodiments 1-47, wherein the therapeutic oil blend does not comprise a water phase.
49. The therapeutic oil blend of any one of embodiments 1-48, wherein the therapeutic oil blend comprises less than 20% water-soluble ingredients.
50. The therapeutic oil blend of any one of embodiments 1-49, wherein the therapeutic oil blend comprises less than 5% water-soluble ingredients.
51. The therapeutic oil blend of any one of embodiments 1-50, wherein the therapeutic oil blend does not comprise any emulsifiers, surfactants, or other homogenizing agents, other than those naturally occurring in hemp oil, clove bud oil, and peppermint oil.
52. The therapeutic oil blend of any one of embodiments 1-51, wherein the therapeutic oil blend has an average particle size of less than 1 μm.
53. The therapeutic oil blend of any one of embodiments 1-52, wherein the therapeutic oil blend has an average particle size of less than 0.1 μm.
54. The therapeutic oil blend of any one of embodiments 1-53, wherein the therapeutic oil blend has an average particle size of about 20-60 nm.
55. The therapeutic oil blend of any one of embodiments 1-54, wherein the therapeutic oil blend is nano-penetrative.
56. The therapeutic oil blend of any one of embodiments 1-55, wherein the therapeutic oil blend penetrates the sensory nerve fibers faster than a corresponding control formulation without the essential oil or terpene component or a corresponding control formulation without the mechanical agitation employed in preparing the therapeutic oil blend.
57. The therapeutic oil blend of any one of embodiments 1-56, wherein the therapeutic oil blend has improved topical absorption compared to a corresponding control formulation without the essential oil or terpene component or a corresponding control formulation without the mechanical agitation employed in preparing the therapeutic oil blend.
58. The therapeutic oil blend of any one of embodiments 1-57, wherein the therapeutic oil blend has anesthetic properties.
59. The therapeutic oil blend of any one of embodiments 1-58, wherein the therapeutic oil blend has anesthetic properties greater than the anesthetic properties of a corresponding control formulation without the essential oil or terpene component.
60. The therapeutic oil blend of any one of embodiments 1-59, wherein the therapeutic oil blend has anesthetic properties greater than the anesthetic properties of a mixture of the components without the mechanical agitation employed in preparing the therapeutic oil blend.
61. The therapeutic oil blend of any one of embodiments 1-60, wherein a topical application of the therapeutic oil blend results in a decrease in painful stimulus at the site of application for a period of at least ten minutes following application.
62. The therapeutic oil blend of any one of embodiments 1-61, wherein the therapeutic oil blend has analgesic properties.
63. The therapeutic oil blend of any one of embodiments 1-62, wherein the therapeutic oil blend has vasodilatory effects.
64. The therapeutic oil blend of any one of embodiments 1-63, wherein topical application of the therapeutic oil blend increases skin temperature at the site of application by at least 0.5° C. over the course of one minute following application.
65. The therapeutic oil blend of any one of embodiments 1-64, wherein topical application of the therapeutic oil blend increases skin temperature at the site of application by at least 1.0° C. over the course of one minute following application.
66. The therapeutic oil blend of any one of embodiments 1-65, wherein the therapeutic oil blend has a faster solvation rate in a carrier oil than solid or liquid hemp oil alone.
67. The therapeutic oil blend of any one of embodiments 1-66, wherein the therapeutic oil blend has a faster solvation rate in MCT oil than solid or liquid hemp oil alone.
68. The therapeutic oil blend of any one of embodiments 1-67, wherein the therapeutic oil blend comprises about 20%-40% w/w eugenol.
69. The therapeutic oil blend of any one of embodiments 1-68, wherein the therapeutic oil blend comprises about 5%-20% w/w menthol and/or menthone; and
70. The therapeutic oil blend of any one of embodiments 1-69, wherein the therapeutic oil blend comprises about 0.1%-10% w/w d-limonene.
71. The therapeutic oil blend of any one of embodiments 1-70, wherein the therapeutic oil blend comprises about 0.01%-5% w/w of one or more terpenes selected from the list consisting of bisabolol, borneol, caryophyllene, carene, camphene, camphor, cineol, citronellal, eucalyptol, geraniol, guaiol, humulene, isopropyltoluene, isopulegol, linalool, beta-myrcene, nerolidol, ocimene, alpha-pinene, beta-pinene, phytol, pulegone, alpha-terpinene, gamma-terpinene, terpinolene, thymol.
72. The therapeutic oil blend of any one of embodiments 1-71, wherein the therapeutic oil blend is formulated for transdermal penetration and/or membrane penetration.
73. The therapeutic oil blend of any one of embodiments 1-72, wherein the therapeutic oil blend is non-crystallizing.
74. The therapeutic oil blend of any one of embodiments 1-73, wherein the therapeutic oil blend is non-crystallizing for a period of at least 30 days.
75. The therapeutic oil blend of any one of embodiments 1-74, wherein the therapeutic oil blend has improved bioavailability, consistency, shelf life, and non-crystallization compared to the cannabinoid oil alone.
76. A mechanically agitated, non-crystallizing liquid therapeutic oil blend with emulsion-like properties comprising:

a) about 63% w/w cannabinoid oil;

b) about 27% w/w clove bud oil; and

c) about 10% w/w peppermint oil.

77. A mechanically agitated, non-crystallizing liquid therapeutic oil blend with emulsion-like properties comprising:

a) about 50% w/w cannabidiol (CBD);

b) about 20-25% w/w eugenol;

c) about 8-10% w/w menthol and menthone; and

d) about 1-3% w/w d-limonene.

78. A topical formulation comprising the therapeutic oil blend according to any one of embodiments 1-77.
79. The topical formulation of embodiment 78, wherein the topical formulation is a lotion.
80. The topical formulation of embodiment 78, wherein the topical formulation is a body balm.
81. The topical formulation of embodiment 78, wherein the topical formulation is a salve.
82. The topical formulation of embodiment 78, wherein the topical formulation is a massage oil.
83. The topical formulation of embodiment 78, wherein the topical formulation is a topical gel.
84. The topical formulation of embodiment 78, wherein the topical formulation is a moisturizer.
85. The topical formulation of embodiment 78, wherein the topical formulation is a cream.
86. The topical formulation of any one of embodiments 78-85, wherein the topical formulation comprises an essential oil.
87. The topical formulation of any one of embodiments 78-86, wherein the topical formulation comprises a carrier oil.
88. The topical formulation of any one of embodiments 78-87, wherein the topical formulation comprises a humectant.
89. The topical formulation of any one of embodiments 78-88, wherein the topical formulation comprises an emulsifier.
90. An ingestible formulation comprising the therapeutic oil blend of any one of embodiments 1-77.
91. The ingestible formulation of embodiment 90, wherein the ingestible formulation is a tablet.
92. The ingestible formulation of embodiment 90 or 91, wherein the ingestible formulation is a pressed tablet.
93. The ingestible formulation of embodiment 90, wherein the ingestible formulation is a tincture.
94. The ingestible formulation of embodiment 90, wherein the ingestible formulation is a drink mix.
95. The ingestible formulation of any one of embodiments 90-94, wherein the ingestible formulation comprises an essential oil.
96. The ingestible formulation of any one of embodiments 90-95, wherein the ingestible formulation comprises a carrier oil.
97. A method of using a therapeutic oil blend according to any of embodiment 1-77, or composition comprising, in the treatment of a condition.
98. A method of using a topical formulation according to any one of embodiments 78-89 or an ingestible formulation according to any one of embodiments 90-97 in the treatment of a condition.
99. The method of embodiment 97 or 98, wherein the condition is pain.
100. The method of any one of embodiments 97-99, wherein the condition is poor sleep quality.
101. The method of any one of embodiments 97-100, wherein the condition is fatigue.
102. The method of any one of embodiments 97-101, wherein the condition is a psychological condition.
103. A hermetically sealed container comprising the therapeutic oil blend of any one of embodiments 1-77, or a composition comprising.
104. A method of formulating a therapeutic oil blend of any one of embodiments 1-77.
105. A method for preparing a mechanically agitated, non-crystallizing therapeutic oil blend with emulsion-like properties, the method comprising the steps of:

a) providing a cannabinoid oil, a clove bud oil, and a peppermint oil ingredients;

b) admixing the ingredients of step (a), thereby forming an inactive mixture; and

c) mechanically agitating the inactive mixture of step (b) until homogeneous, thereby producing the mechanically agitated therapeutic oil blend.

106. A method for preparing a sonicated, non-crystallizing therapeutic oil blend with emulsion-like properties, the method comprising the steps of:

a) providing a cannabinoid oil, a clove bud oil, and a peppermint oil ingredient;

b) admixing the ingredients of step (a), thereby forming an inactive mixture; and

c) sonicating the inactive mixture of step (b) until homogeneous, thereby producing the sonicated therapeutic oil blend.

107. A method for preparing a mechanically agitated therapeutic oil blend that is liquid at room temperature and does not crystallize at room temperature for a period of at least 60 days, the method comprising the steps of:

a) providing a cannabinoid oil, a clove bud oil, and a peppermint oil ingredient;

b) admixing the ingredients of step (a), thereby forming an inactive mixture; and

c) mechanically agitating the inactive mixture of step (b) until homogeneous, thereby producing the mechanically agitated therapeutic oil blend.

108. A method for preparing a mechanically agitated therapeutic oil blend with emulsion-like properties that has an average particle size of less than 0.1 μM, the method comprising the steps of:

a) providing a cannabinoid oil, a clove bud oil, and a peppermint oil ingredient;

b) admixing the ingredients of step (a), thereby forming an inactive mixture; and

c) mechanically agitating the inactive mixture of step (b) until homogeneous, thereby producing the mechanically agitated therapeutic oil blend.

109. A method for preparing a mechanically agitated, non-crystallizing therapeutic oil blend with emulsion-like properties, the method comprising the steps of:
a) providing a cannabinoid oil, eugenol, menthol, menthone, and d-limonene ingredients;
b) admixing the ingredients of step (a), thereby forming an inactive mixture; and
c) mechanically agitating the inactive mixture of step (b) until homogeneous, thereby producing the mechanically agitated therapeutic oil blend.
   110. A method for preparing a sonicated, non-crystallizing therapeutic oil blend with emulsion-like properties, the method comprising the steps of:
a) providing a cannabinoid oil, eugenol, menthol, menthone, and d-limonene ingredient;
b) admixing the ingredients of step (a), thereby forming an inactive mixture; and
c) sonicating the inactive mixture of step (b) until homogeneous, thereby producing the sonicated therapeutic oil blend.
   111. A method for preparing a mechanically agitated therapeutic oil blend that is liquid at room temperature and does not crystallize at room temperature for a period of at least 60 days, the method comprising the steps of:
a) providing a cannabinoid oil, eugenol, menthol, menthone, and d-limonene ingredient;
b) admixing the ingredients of step (a), thereby forming an inactive mixture; and
c) mechanically agitating the inactive mixture of step (b) until homogeneous, thereby producing the mechanically agitated therapeutic oil blend.
   112. A method for preparing a mechanically agitated therapeutic oil blend with emulsion-like properties that has an average particle size of less than 0.1 μM, the method comprising the steps of:
a) providing a cannabinoid oil, eugenol, menthol, menthone, and d-limonene ingredient;
b) admixing the ingredients of step (a), thereby forming an inactive mixture; and
c) mechanically agitating the inactive mixture of step (b) until homogeneous, thereby producing the mechanically agitated therapeutic oil blend.
   113. The method according to any one of embodiments 105-112, wherein the cannabinoid oil ingredient has all of its cannabinoid contents fully dissolved before the admixing step.
   114. The method according to any one of embodiments 105-113, wherein the cannabinoid oil ingredient has been cavitated before the admixing step.
   115. The method according to any one of embodiments 105-114, wherein the cannabinoid oil is heated until fully liquified prior to admixing or prior to mechanical agitation.
   116. The method according to any one of embodiments 105-115, wherein the cannabinoid oil is heated to at least 60° C. prior to admixing or prior to mechanical agitation.
   117. The method according to any one of embodiments 105-116, wherein the method comprises mechanically agitating the cannabinoid oil ingredient prior to step (b).
   118. The method according to any one of embodiments 105-117, wherein the method further comprises the following steps prior to step (b):

i) heating the cannabinoid oil to at least 60° C. until fully liquified, and

ii) mechanically agitating the liquified cannabinoid oil.

119. The method according to any one of embodiments 105-118, wherein the cannabinoid oil is mechanically agitated until cavitation is induced prior to step (b).
120. The method according to any one of embodiments 105-119, wherein step (b) comprises mechanically agitating the inactive mixture until a sufficient level of cavitation is induced so as to fully homogenize the mixture.
121. The method according to any one of embodiments 105-120, wherein the temperature of the cannabinoid oil does not exceed about 80° C. for more than 10, 20, 30, 40, 50, 60, 70, 80, 90 or 100 minutes during any step carried out on the cannabinoid oil alone.
122. The method according to any one of embodiments 105-121, wherein the inactive mixture is maintained below a temperature of about 60° C. throughout step (c).
123. The method according to any one of embodiments 105-122, wherein the inactive mixture is maintained below a temperature of about 55° C. throughout step (c).
124. The method according to any one of embodiments 105-123, wherein the inactive mixture is maintained below a temperature of about 40° C. throughout step (c).
125. The method according to any one of embodiments 105-124, wherein the ingredients other than the cannabinoid oil are chilled prior to step (c).
126. The method according to any one of embodiments 105-125, wherein the ingredients other than the cannabinoid oil are chilled to about less than 0° C. prior to step (c).
127. The method according to any one of embodiments 105-126, wherein the cannabinoid oil is at room temperature prior to adding other ingredients in step (b).
128. The method according to any one of embodiments 105-127, wherein step (b) and (c) are carried out such that the inactive mixture remains at or below 25° C.
129. The method according to any one of embodiments 105-128, wherein step (b) and (c) comprise sequentially adding each ingredient to the cannabinoid oil, with a round of mechanical agitation in between the addition of each ingredient.
130. The method according to any one of embodiments 105-129, wherein the eugenol or the clove bud oil is added to the cannabinoid oil, thereby producing a eugenol mixture, prior to addition of the other ingredients in step (b).
131. The method according to any one of embodiments 105-130, wherein the eugenol mixture is mechanically agitated prior to addition of the other ingredients in step (b).
132. The method according to any one of embodiments 105-131, wherein the temperature of the eugenol mixture is maintained below about 60° C. during mechanical agitation.
133. The method according to any one of embodiments 105-132, wherein the eugenol or clove bud oil is added to the cannabinoid oil while the cannabinoid oil is experiencing mechanical agitation.
134. The method according to any one of embodiments 105-133, wherein the eugenol or clove bud oil is added to the cannabinoid oil while the cannabinoid oil is experiencing sonication.
135. The method according to any one of embodiments 105-134, wherein the mechanical agitation is carried out at settings selected to avoid vaporization, sublimation, or denaturation of the eugenol.
136. The method according to any one of embodiments 105-135, wherein the other ingredients are added to the mechanically agitated eugenol mixture, thereby producing an overall mixture.
137. The method according to any one of embodiments 105-136, wherein the mechanically agitated eugenol mixture is chilled to less than about 15° C. prior to addition of the remaining ingredients.
138. The method according to any one of embodiments 105-137, wherein the other ingredients are chilled prior to being added to the mechanically agitated eugenol mixture.
139. The method according to any one of embodiments 105-138, wherein the temperature of the other ingredients and the temperature of the mechanically agitated eugenol mixture are such that the temperature of the inactive mixture is kept below about 15° C.
140. The method according to any one of embodiments 105-139, wherein the inactive mixture is mechanically agitated.
141. The method according to any one of embodiments 105-140, wherein the temperature of the inactive mixture is maintained below about 40° C. during mechanical agitation.
142. The method according to any one of embodiments 105-141, wherein the other ingredients are added to the eugenol mixture to form an inactive mixture during the process of mechanical agitation.
143. The method according to any one of embodiments 105-142, wherein steps (b) and (c) are conducted simultaneously.
144. The method according to any one of embodiments 105-143, wherein the mechanical agitation is carried out at settings selected to avoid vaporization, sublimation, or denaturation of the eugenol and other ingredients.
145. The method according to any one of embodiments 105-144, wherein d-limonene is added to the eugenol mixture prior to the addition of menthol and menthone.
146. The method according to any one of embodiments 105-145, wherein following step (c), the inactive mixture is further mechanically agitated.
147. The method according to any one of embodiments 105-146, wherein the temperature does not exceed about 55° C. during further mechanical agitation.
148. The method according to any one of embodiments 105-147, wherein the mechanical agitation step is carried out until homogeneity is achieved.
149. The method according to any one of embodiments 105-148, wherein the mechanical agitation induces cavitation.
150. The method according to any one of embodiments 105-149, wherein the mechanical agitation is sonication.
151. A method for preparing a mechanically agitated, non-crystallizing, homogeneous therapeutic oil blend with emulsion-like properties, wherein the therapeutic oil blend comprises 25-75% w/w cannabinoid; 15%-40% w/w eugenol; 5%-20% w/w menthol and/or menthone; and 0.1%-10% w/w d-limonene, the method comprising the steps of
a) providing a cannabinoid oil comprising the cannabinoid, heating said cannabinoid oil to at least about 60° C., and sonicating said cannabinoid oil to induce cavitation;
b) adding the eugenol to the sonicated cannabinoid oil of (a) and sonicating until the mixture is fully homogenized;
c) adding the d-limonene, menthol, and/or menthone to the mixture of (b) and sonicating until the mixture is fully homogenized;
d) sonicating the mixture of (c) until the solution has the desired particle size, thereby preparing the therapeutic oil blend.
   152. A method for preparing a mechanically agitated, non-crystallizing, homogeneous therapeutic oil blend with emulsion-like properties, wherein the therapeutic oil blend comprises 25-75% w/w cannabinoid; 15%-40% w/w eugenol; 5%-20% w/w menthol and/or menthone; and 0.1%-10% w/w d-limonene, the method comprising the steps of
a) providing a cannabinoid oil comprising the cannabinoid, heating said cannabinoid oil to at least about 60° C., and sonicating said cannabinoid oil to induce cavitation, wherein the temperature does not exceed about 80° C. during sonication;
b) adding the eugenol to the sonicated cannabinoid oil of (a) and sonicating until the mixture is fully homogenized, wherein the temperature does not exceed about 60° C. during sonication;
c) adding the d-limonene, menthol, and/or menthone to the mixture of (b) and sonicating until the mixture is fully homogenized, wherein the temperature does not exceed about 40° C. during sonication;
d) sonicating the mixture of (c) until the solution has the desired particle size,
wherein the temperature does not exceed about 55° C. during sonication,
   thereby preparing the therapeutic oil blend.
   153. A method for preparing a sonicated, non-crystallizing therapeutic oil blend with emulsion-like properties, wherein the therapeutic oil blend comprises 30-90% w/w cannabidiol-related cannabinoid oil, 15-40% clove bud oil, and 5-20% peppermint oil, the method comprising the steps of:
a) providing a fully liquified cannabidiol-related cannabinoid oil, a clove bud oil, and a peppermint oil;
b) sonicating the fully liquified cannabidiol-related cannabinoid oil to induce cavitation, wherein the temperature of the cannabidiol-related cannabinoid oil does not exceed about 80° C. during sonication, thereby producing a sonicated cannabinoid oil;
c) adding chilled clove bud oil to the sonicated cannabinoid oil and sonicating this mixture, thereby producing a sonicated eugenol mixture, wherein the temperature of the sonicated eugenol mixture does not exceed about 60° C. during sonication, and wherein the clove bud oil is added during a sonication step;
d) adding chilled peppermint oil to the sonicated eugenol mixture and sonicating this mixture at low power, thereby producing a sonicated peppermint mixture, wherein the temperature of the sonicated peppermint mixture does not exceed about 40° C. during sonication, and wherein the peppermint oil is added during a sonication step; and
e) further sonicating the sonicated peppermint mixture at high power to produce a sonicated therapeutic oil blend, wherein the temperature of the sonicated therapeutic oil blend does not exceed about 55° C. during sonication, thereby producing the sonicated therapeutic oil blend.
   154. A method according to any one of embodiments 105-153, wherein the method produces a therapeutic oil blend according to any one of embodiments 1-77.

1-68. (canceled)

69. A cavitated therapeutic oil blend comprising:
a) 25-75% w/w cannabinoid content; and
b) 25-75% w/w terpene content,
wherein the cavitated therapeutic oil blend has an average cannabinoid crystal size of less than 300 nm at room temperature, wherein the cavitated therapeutic oil blend comprises cavitation bubbles, and wherein weight contents are calculated based on the weight of the cavitated therapeutic oil blend.

70. The cavitated therapeutic oil blend of claim 69, wherein the average cannabinoid crystal size remains below 300 nm for a period of at least 14 days.

71. The cavitated therapeutic oil blend of claim 69, wherein the cavitated therapeutic oil blend is not an emulsion.

72. The cavitated therapeutic oil blend of claim 69, wherein the cavitated therapeutic oil blend: does not comprise a water phase; or comprises less than 5% water content by weight.

73. The cavitated therapeutic oil blend of claim 69, wherein the cavitated therapeutic oil blend does not comprise any emulsifiers, surfactants, or homogenizing agents, other than those naturally occurring in hemp oil, clove bud oil, and peppermint oil.

74. The cavitated therapeutic oil blend of claim 69, wherein the cavitated therapeutic oil blend has an average cannabinoid crystal size of less than about 60 nm at room temperature.

75. The cavitated therapeutic oil blend of claim 69, wherein the cannabinoid is selected from the group consisting of: cannabidiol (CBD), cannabidiolic acid (CBDA), cannabidiol monomethylether (CBDM), cannabidiol-C4 (CBD-C4), cannabidivarinic acid (CBDVA), cannabidivarin (CBDV), cannabidiorcol (CBD-C1), delta 9 tetrayhydrocannabinol (THC), and a combination thereof.

76. The cavitated therapeutic oil blend of claim 69, wherein the cannabinoid is cannabidiol (CBD).

77. The cavitated therapeutic oil blend of claim 69, wherein the cavitated therapeutic oil blend comprises about 15%-40% w/w eugenol.

78. The cavitated therapeutic oil blend of claim 69, wherein the cavitated therapeutic oil blend comprises about 5%-20% w/w menthol, 5%-20% w/w menthone, or a combination thereof.

79. The cavitated therapeutic oil blend of claim 69, wherein the cavitated therapeutic oil blend comprises about 0.1%-10% w/w d-limonene.

80. The cavitated therapeutic oil blend of claim 69, wherein the cavitated therapeutic oil blend comprises about 0.01%-5% w/w of a terpene selected from the list consisting of: bisabolol, borneol, caryophyllene, carene, camphene, camphor, cineol, citronellal, eucalyptol, geraniol, guaiol, humulene, isopropyltoluene, isopulegol, linalool, beta-myrcene, nerolidol, ocimene, alpha-pinene, beta-pinene, phytol, pulegone, alpha-terpinene, gamma-terpinene, terpinolene, and thymol.

81. The cavitated therapeutic oil blend of claim 69, wherein the terpene content comprises an essential oil.

82. The cavitated therapeutic oil blend of claim 81, wherein the essential oil comprises clove bud oil or peppermint oil.

83. The cavitated therapeutic oil blend of claim 69, wherein the cavitated therapeutic oil blend comprises about 15-40% w/w clove bud oil.

84. The cavitated therapeutic oil blend of claim 69, wherein the cavitated therapeutic oil blend comprises about 5-20% w/w peppermint oil.

85. The cavitated therapeutic oil blend of claim 69, wherein the cavitated therapeutic oil blend has increased topical absorption as compared to either:
a) a control un-cavitated formulation comprising all ingredients of the therapeutic oil blend, but no cavitation bubbles or;
b) a control cavitated formulation without the terpene content.

86. The cavitated therapeutic oil blend of claim 69, wherein the cavitated therapeutic oil blend has increased anesthetic, analgesic, and/or vasodilatory properties as compared to either:
a) a control un-cavitated formulation comprising all ingredients of the therapeutic oil blend, but no cavitation bubbles or;
b) a control cavitated formulation without the terpene content.

87. The cavitated therapeutic oil blend of claim 69, wherein the cavitated therapeutic oil blend cavitation was induced through mechanical agitation.

88. The cavitated therapeutic oil blend of claim 87, wherein the mechanical agitation comprises sonication.

89. A cavitated therapeutic oil blend that comprises:
a) 25-90% w/w cannabidiol content;
b) 25%-75% w/w terpene content, wherein the terpene content comprises:
i) clove bud oil;
ii) peppermint oil; or
iii) both i) and ii),
wherein the cavitated therapeutic oil blend has an average cannabinoid crystal size of less than 300 nm at room temperature, wherein the cavitated therapeutic oil blend comprises cavitation bubbles, and wherein weight contents are calculated based on the weight of the cavitated therapeutic oil blend.

90. A topical formulation comprising the therapeutic oil blend of claim 69, said topical formulation selected from the group consisting of: a lotion, a body balm, a salve, a massage oil, a topical gel, a moisturizer, and a cream.

91. An ingestible formulation comprising the therapeutic oil blend of claim 69, said ingestible formulation selected from the group consisting of: a tablet, a pressed tablet, a tincture, and a drink mix.

92. A method of preparing a cavitated therapeutic oil blend, the method comprising:
a) heating a cannabinoid under conditions sufficient to melt or dissolve the cannabinoid, thereby producing a liquid cannabinoid;
b) admixing:
i) 25-75% w/w liquid cannabinoid; and
ii) 25-75% w/w terpene, thereby producing a mixture; and
c) cavitating the mixture of step (b) under conditions sufficient to generate cavitation bubbles, thereby generating the cavitated therapeutic oil blend, wherein the cavitated therapeutic oil blend has an average cannabinoid crystal size of less than 300 nm at room temperature, and wherein weight contents are calculated based on the weight of the cavitated therapeutic oil blend.

93. The method of claim 92, wherein the mixture is kept below a temperature of about 60° C. during step (c).
